(19) United States
(12) Patent Application Publication
(10) Pub. No.: US 2021/0207196 A1
(43) Pub. Date: Jul. 8, 2021

(54) QUALITY CONTROL COMPOSITIONS AND WHOLE ORGANISM CONTROL MATERIALS FOR USE IN NUCLEIC ACID TESTING

(71) Applicant: MICROBIX BIOSYSTEMS INC., Mississauga (CA)

(72) Inventors: Mark A LUSCHER, Toronto (CA); Kenneth Hughes, Toronto (CA); Pavel Zhelev, Toronto (CA); Cameron Groome, Mississauga (CA)

(21) Appl. No.: 17/250,065

(22) PCT Filed: May 21, 2019
(86) PCT No.: PCT/CA2019/050689

§ 371 Date: Nov. 18, 2020

Related U.S. Application Data
(60) Provisional application No. 62/673,480, filed on May 18, 2018.

(51) Int. Cl. C12Q 1/689; C12Q 1/6844

(52) U.S. Cl. CPC C12Q 1/689; C12Q 2600/106; C12Q 1/6844

(57) ABSTRACT

The present disclosure relates in general, to quality control compositions and whole organism control materials for use in assessing the functionality of Nucleic Acid Tests (NAT) and to related methods, uses and kits. In some aspects the NAT is used to detect the presence of an organism and a target nucleic acid sequence in a test article, the quality control composition comprising: (i) a first protected nucleic acid sequence comprising a first nucleic acid sequence the presence of which is characteristic of the presence of the organism in the test article said first nucleic acid sequence being tested in the NAT; and (ii) a second protected nucleic acid sequence comprising a second nucleic acid sequence the presence of which is characteristic of the presence of the target nucleic acid sequence in the test article said second nucleic acid sequence being tested for in the NAT.

### CROSS REFERENCE

This application claims priority from US Provisional Application U.S. 62/673,480, Filed May 18, 2018, entitled, Whole Organism Control Materials for Use in Nucleic Acid Testing, the entirety of which is herein incorporated by reference. The relevant PCT receiving office was closed for business on May 18-20, 2019, inclusive.

### FIELD OF THE DISCLOSURE

The present disclosure relates, in general, to compositions of Whole Organism Control Materials for Nucleic Acid Tests (NAT), to a quality control composition used to assess the functionality of a NAT and methods for detecting an organism or nucleic acid sequence of interest in a test sample, such as a biological test sample.

### BACKGROUND OF THE DISCLOSURE

### Nucleic Acid Test and Quality Control

Analytical and/or diagnostic testing for the presence of a biological organism or trait may be performed utilizing methods that detect the nucleic acids, or the nucleic acid sequences, of the organism, broadly referred to as Nucleic Acid Tests (NAT). One subset of Nucleic Acid Tests is known as Nucleic Acid Amplification Tests (NAAT), as these most often involve the amplification of the nucleic acid to facilitate detection. Such methods, for example, may be based on the detection of DNA or of RNA sequences characteristic of the organism or to a specific trait or feature. NAT can be used to identify the presence of a particular nucleic acid sequence or sequences that are characteristic of the presence or absence of a genetic condition or trait or feature, or of organisms, such as of bacteria, viruses, or of pathogens or other species. Examples include the detection of antibiotic resistant bacteria or malaria or respiratory viruses (e.g., influenza, RSV) in a biological sample, such as bodily fluid or blood or urine or tissue. In other aspects, NAT can be used to detect the presence of antibiotic resistance (such as methicillin resistance in methicillin-resistant Staphylococcus Aureus, MRSA), diseases (such as malaria, or hypervirulent influenza), and contaminants or pests, such as E. coli levels in water, or fungal spores in soil (e.g. pore suspensions of Bacillus subtilis, or molds).

Quality Control in NAT is increasingly important, as detection technologies become more sensitive, results are influenced not only by biological variability (e.g., samples or sample types) but also by technical variability, e.g., any differences introduced during sample processing, variations in critical reagents, control of contamination in the testing environment, procedural consistency, and the proper functioning of the test equipment. The more processing steps, the higher the risk of generating false positives or false negatives. False results can have very detrimental consequences, not only in the cost of performing the tests (or reperforming), but also the cost to health and safety—whether it be to a patient, or the environment or other system being tested. Given the multi-step nature of many NAT, there is a need for quality control materials that can be used as a control for the entire process.

Further, there is a need to develop non-pathogenic and/or non-virulent and/or non-toxic quality controls for pathogenic and/or virulent and/or toxic organisms that are safe to produce, transport and handle.

### Controls

Samples where the presence or absence of an analyte is known, and which are used to design or develop a test, to establish proper functioning of the test (for example, to challenge, confirm or assess the operation, performance, equipment, systems or procedures associated with the test, to provide evidence of the proper functioning and/or the validity of the results of the test, whether required by regulation, law, or principles of best practice), are generally referred to and as “Controls”.

Controls may be extrinsic to the test articles of interest or may be tested independently of the test articles of interest, in which case they may be referred to as ‘External Controls’ or ‘Extrinsic Controls’. Alternatively, the Controls may be integrated with or mixed with the test article of interest, in which case they may be referred to as ‘Internal Controls’. Controls may be qualitative, in that they exemplify the presence or absence of the analyte, or they may be quantitative, in which case they contain a predetermined absolute or relative quantity of the analyte. The FDA and other health authorities have specified the need for both negative and positive NAT controls. See for instance, U.S. FDA Guidance document on “Class II Special Controls Guidance Document: Respiratory Viral Panel Multiplex Nucleic Acid Assay—Guidance for Industry and FDA Staff” (October 2009) [https://www.fda.gov/requlatory-information/search-fda-quidance-documents/class-ii-special-controls-guidance-document-respiratory-viral-panel-multiplex-nucleic-acid-assay#5]

Negative Controls can sometimes be provided as a buffer or a sample matrix or storage material, substantially without organism or genetic material that is the specific target of the NAT. In other embodiments, there could be a negative-sample control containing non-target nucleic acid or a whole organism not targeted by the assay. One example is a patient specimen from a known non-infected individual, a non-target organism or a surrogate negative control, e.g. packaged RNA (or armored RNA). There are many reasons for performing negative controls, including but not limited to the demonstration of the lack of nucleic acid contamination of test components, equipment and the environment by nucleic acids that could falsely render a test result positive and the demonstration that the test does not cross-react with irrelevant materials or include intrinsic elements that might otherwise render a positive result in the absence of a test article bearing the specific target sequence.

The positive control is designed to mimic some aspect of a specimen, contains target nucleic acids, and may be used to control all or part of the assay process, including nucleic acid extraction, amplification, and detection. Useful positive assay control materials include patient samples, cell lines, cell lines infected with pathogenic or non-pathogenic strains of pathogens detected by the assay, purified or isolated pathogens, nucleic acids from pathogens or cell lines, and nucleic acids presented in an enclosing organism or structure, any of which is presented in or on the appropriate matrix mimicking in some aspects the assay specimen type. Armored RNA and DNA are examples of enclosed nucleic acids that may be used as positive controls. Positive controls may be run as a separate assay or in some cases concurrently with specimen samples. A positive control may also be a target nucleic acid that is otherwise unrelated to the specific target of the assay but is a secondary assay target included for the purpose of determining the integrity of the reagents (e.g., polymerase, primers), equipment function (e.g., thermal cycler), and the presence of inhibitors in the samples or for supporting a qualitative or quantitative result of the assay. Such controls include ‘internal controls’, for example the internal controls described in U.S. Pat. No. 9,328,385. Further examples include human nucleic acid co-extracted with the influenza virus and primers amplifying human housekeeping genes (e.g., RNase P, β-actin).

A NAT for the presence and/or quantitation of organisms may be designed to detect a complete or partial quantity of the nucleic acid of an organism, so Controls for such tests can be derived from a variety of sources or organisms.

There is a high need for developing such Controls, especially Controls that are easier/safer to manufacture or handle and that better simulate the effect of the NAT from processing to nucleic acid detection on the test example.

Further, the preparation of appropriate control materials presents unsolved problems. One such problem is how to prepare Controls that are broadly useful in many different tests, whether developed by commercial manufactures of NAT, including diagnostic NAT, as well as by individual research or clinical laboratories. For instance, many Controls are useful for only a particular NAT or set of NAT but not all NAT for a particular organism.

Therefore it will be generally known to those skilled in the art that there is an unmet need for methods to produce controls that have at least some of the following features: (i) Controls that are recognized by many NAT of different design and origin (e.g. that are more universal or have broader applications), (ii) are readily obtained, (iii) are readily cultured or grown without undue cost or biosafety risk, and (iv) can be rapidly developed in response to the need to provide controls as sequences of interest evolve.

### SUMMARY

The present invention provides, among other benefits, the ability to meet one or more of the needs noted above and to provide other desirable features of controls as described herein.

In various aspects of the invention, the invention provides a quality control composition used to assess the functionality of a Nucleic Acid Test (NAT), wherein the NAT is used to detect the presence of an organism and a target nucleic acid sequence in a test article, the quality control composition comprising: (i) a first protected nucleic acid sequence comprising a first nucleic acid sequence the presence of which is characteristic of the presence of the organism in the test article said first nucleic acid sequence being tested in the NAT; and (ii) a second protected nucleic acid sequence comprising a second nucleic acid sequence the presence of which is characteristic of the presence of the target nucleic acid sequence in the test article said second nucleic acid sequence being tested for in the NAT. In some aspects, the first and second protected nucleic acid sequences are together in one or separately in more than one protecting vehicle, wherein the protecting vehicles can be the same or different type of protecting vehicle. In another aspect, the second protected nucleic acid sequence is not natively present in its protecting vehicle.

In various embodiments, the protecting vehicle of the first nucleic acid sequence is an organism that has the same or similar nucleic acid sequence protection properties when processed through the NAT as the organism or organism comprising the target sequence the presence of which is to be tested for in the test article. In some embodiments said protecting vehicle of the first nucleic acid sequence does not comprise the target nucleic acid sequence.

In some other aspects the protecting vehicles of the composition of the invention protect their respective nucleic acid sequences so that the nucleic acid sequences can be sufficiently, and/or substantially recovered in the nucleic acid extraction steps of the NAT.

In some other aspects, at least one of the protecting vehicles, such as for the first protected nucleic acid sequence, is an organism that has the same or similar nucleic acid protection properties when processed through the NAT as the organism the presence of which is being tested for in the sample or test article. In other various embodiments, the protecting vehicle of the first protected nucleic acid sequence is the organism that is being tested for in the NAT without the target nucleic acid sequence and the first nucleic acid sequence is the native genome of the organism or an organism of the organism family that does not comprise the target nucleic acid sequence.

In various embodiments of the invention: (i) the organism comprising the target nucleic acid sequence to be tested for in the NAT is a virulent or pathogenic or toxic organism or wherein when the target nucleic acid sequence is present with or without additional sequences a virulent or pathogenic or toxic trait is conferred on the organism; and (ii) the protecting vehicle of the first protected nucleic acid sequence is a less- or non-virulent and/or pathogenic form of the organism or an organism of the organism family comprising a native genomic nucleic acid sequence that does not comprise the target nucleic acid sequence. In some other aspects, the organism comprising the target nucleic acid sequence to be tested for in the NAT is a particular strain or set of strains belonging to a species or genus of an organism, wherein the protecting vehicle of the first protected nucleic acid sequence is a representative strain of the species or genus comprising a native genomic nucleic acid sequence but not the target nucleic acid sequence.

In some other aspects of the invention in the quality control composition the second nucleic acid sequence is: (i) selected from a portion of the nucleic acid sequence characteristic of the target nucleic acid sequence, or (ii) is modified, such that the second nucleic acid sequence would not be expressed to confer the expression of the genetic feature of the target sequence, either alone or in combination with other nucleic acid sequences in the composition.

In some other aspects and embodiments, the quality control composition comprises more than one first nucleic acid sequences, and each first nucleic acid sequence being a different nucleic acid sequence (overlapping or non-overlapping for a region of the target nucleic acid sequence) characteristic of the organism in the test article, at least one but not necessarily all, of the first nucleic acid sequences being tested for in a NAT. In some aspects, the first nucleic acid sequences are each in a separate protecting vehicle, in more than one protecting vehicle or in one protecting vehicle.

In some other various embodiments where there are more than one first nucleic acid sequences, more than one of the first nucleic acid sequences are tested for in a NAT and the presence of more than one of the first nucleic acid sequences is necessary to determine the presence of the organism.

Similarly, in various embodiments of the invention wherein the composition comprises, more than one second nucleic acid sequences, each second nucleic acid sequence being different nucleic acid sequences (overlapping or non-overlapping for a region of the target nucleic acid sequence) characteristic of the presence of the target nucleic acid sequence, at least one but not necessarily all, of the second nucleic acid sequences being tested for in a NAT. In some aspects, the second nucleic acid sequences are each in a separate protecting vehicle, in more than one protecting vehicles, or in one protecting vehicle. In some other various embodiments where there are more than one second nucleic acid sequences, more than one of the second nucleic acid sequences are tested for in a NAT and the presence of more than one of the second nucleic acid sequences is necessary to determine the presence of the test article in the NAT.

In various embodiments, the one or more first protecting vehicles comprising one or more first nucleic acid sequences, comprise alone or together 0.5 to 100% of the genome native to the organism to be tested for in the NAT, or greater than 90%, or greater than 80%, or greater than 70%, or greater than 60%, or greater than 50%, or greater than 40%, or greater than, 30%, or greater than 20%, or greater than 15%, or greater than 10%, or greater than 5%, or greater than 4%, or greater than, 3%, or greater than 2%, or greater than 1%.

In various embodiments, the one or more second protecting vehicles comprising one or more second nucleic acid sequences, comprise alone or together 0.5 to 100% of the target nucleic acid to be tested for in the NAT, or greater than 90%, or greater than 80%, or greater than 70%, or greater than 60%, or greater than 50%, or greater than 40%, or greater than, 30%, or greater than 20%, or greater than 15%, or greater than 10%, or greater than 5%, or greater than 4%, or greater than, 3%, or greater than 2%, or greater than 1%.

In various embodiments of the invention, the NAT is designed to test for a pathogenic, virulent or toxic organism or target nucleic acid sequence and quality control composition of the invention does not comprise said pathogenic, virulent or toxic organism or full target nucleic acid sequence or if so, comprises it in a less or non-pathogenic, virulent or toxic form. In the case of nucleic acid sequences in the quality control composition of the invention, they cannot or have lower, minimal or no risk, alone or in combination with other components of the quality control compositions to confer or express pathogenicity, virulence or toxicity or in some embodiments less pathogenicity, virulence or toxicity than the test sample or test article tested for by the NAT. In some other various embodiments, the first and second nucleic acid sequences are in different protecting vehicles, the first nucleic acid sequence is in a protecting vehicle that is a less virulent, less pathogenic form of the organism in the test article comprising native nucleic acid sequence and the second nucleic acid sequence is in a second organism as the protecting vehicle that does not comprise a nucleic acid sequence of the first protected nucleic acid sequence.

In various other embodiments useful when the NAT is used to detect the presence of one or more additional organisms in a test article, the quality control composition of the invention comprising one or more additional controls for the same or different organism or organisms or target nucleic acid sequence or sequences of the NAT. Alternatively, or in addition, the quality control composition of the present invention may comprise a non-NAT based detection system of additional organisms that may be suspected of being in the sample or test article. Also, in addition or alternatively, the quality control composition of the present invention may comprise further control components for the same organism (dual or multi-control), such as an additional quality control materials for a NAT with a different target sequence for the same organism suspected of being in the sample (or test article) or a different nucleic acid test (non-NAT), or for a non-nucleic acid based detection system, e.g. immunoassay, and use of antibodies.

In some other embodiments where the quality control compositions comprises one or more additional controls, such controls are selected from one or more of: (a) one or more quality control compositions or the compositions of same as described herein, wherein the NAT is used to detect the same or different organism or organisms or target nucleic acid sequence or sequences in the test article; (b) one or more organisms or other constructs comprising nucleic acid sequences, the presence of which is being tested for in the NAT; and (c) one or more organisms, antibodies, proteins, lipids, polysaccharides or nucleic acids representing positive control test materials for test being performed concurrently with the NAT.

In various embodiments of the invention, of the quality control composition and methods of the invention the NAT tests for the presence of methicillin-resistant Staphylococcus aureus in the test article, including testing for a nucleic acid sequence that is found generally in a Staphylococcus aureus and for a nucleic acid sequence that is characteristic of the target nucleic acid sequence methicillin resistance, the composition comprising a first and second protected nucleic acid, the first protected nucleic acid comprising a sequence native to Staphylococcus aureus strains that does not express methicillin resistance, and the second protected nucleic acid comprising a sequence characteristic of methicillin resistance, modified or unmodified, but which by itself cannot confer methicillin resistance on its protecting vehicle alone or in combination.

In some embodiments of the invention, the invention provides a method of preparing a quality control composition of the invention comprising the steps preparing the first and second protected nucleic acid sequences by natural or by genetic modification of the protecting vehicle with the first and second nucleic acid sequences, or by physical or chemical means to couple or include the nucleic acid sequences to or within the protecting vehicle. In some aspects, the second nucleic acid sequence is introduced into its protecting vehicle, wherein the protecting vehicle is an organism, with or without integration into the genome sequences of the organism. In some other aspects, the second nucleic acid sequence is contained within a vector and introduced into its protecting vehicle by transient transfection, introduced by viral transduction, or integrated into the genome of the organism.

In some other embodiments, the invention provides a method for assessing the functionality of a NAT, comprising subjecting the quality control composition of the invention to a NAT and determining if the NAT produces a result that is consistent with the presence of the organism that is the target of the NAT, such as determining whether the NAT produces results consistent with the nucleotide sequences known to be present in the quality control composition. In some other aspects, a pre-determined amount of the quality control composition at different levels is used to determine the quantitative results of the NAT and/or the detection parameters of the NAT.

In various other aspects, the invention provides a method of detecting an organism or target nucleic acid sequence in a test sample using NAT comprising: (i) obtaining a test sample suspected of comprising the organism or target nucleic acid sequence of interest; (ii) providing a quality control composition according to the invention optionally in a medium corresponding to a medium of the test article; (iii) performing the Nucleic Acid Tests (NAT) in each of [0001]44(a) and [0001]44(b) in parallel or in sequence; and (iv) comparing the results produced by the NAT for the test article with the results produced by the NAT for the quality control composition to assess the functionality of the NAT and to provide supportive evidence for the accuracy of the results of the NAT for the test article.

In some other aspects, the invention provides a kit for to assess the functionality of a Nucleic Acid Test (NAT) comprising any one or more of the compositions or components of the quality control composition of the invention and optionally, such as one or more of the following directions: for the preparation or selection of materials of the quality control composition, conducting the control tests with NAT, and directions regarding same and how to assess results from the controls and the sample or test article.

In some other embodiments the quality control composition of the invention is a Whole Organism Control for use in a Nucleic Acid Test (NAT) wherein the Whole Organism Control comprises: (a) a less virulent or less pathogenic or less toxic version of the organism for which the sample is to be screened, which is less toxic or less pathogenic or less virulent or dangerous than the organism to be screened for in the NAT; and (b) one or more nucleotide sequences that are determinative of the presence of the virulent, pathogenic or toxic strain of a whole organism containing (i) at least one nucleic acid sequence providing a characteristic of that organism that is tested in a Nucleic Acid Test (NAT), and (ii) a second nucleic acid sequence providing a characteristic not natively present in the organism that is tested in a NAT.

Contemplated in some embodiments of the invention are compositions comprising quality control compositions that are Augmented Whole Organism Control materials that are used in Nucleic Acid Tests (NAT). Such Augmented Whole Organism Controls may contain a single organism or multiple organisms (Multi-Organism Controls) as protecting vehicles. In various embodiments, the Augmented Whole Organism Control materials comprise a whole organism containing at least one nucleic acid sequence, tested in a NAT, providing a characteristic of that organism (i.e. an endogenous nucleic acid sequence), and containing a second nucleic acid sequence, tested in a NAT, that is not natively present in the organism (i.e., an exogenous nucleic acid sequence).

The exogenous nucleic acid sequences are selected from the nucleic acid sequences that are the target nucleic acid sequences in the sample to be tested. (i.e., a positive control).

In some other aspects, when the quality control composition or Augmented Whole Organism Control material comprises one whole organism, the organism comprises at least two nucleic acid sequences tested in a native that is not natively present in the organism.

In some other aspects, the quality control compositions of the invention, such as the Augmented Whole Organism Control, at least one of the organisms is selected from organisms that would be similar to the organism that is present in the sample to be tested. So that it would act as a control for the sample processing steps of the NAT.

As such, in some aspects, the invention provides a NAT Control that in one Whole Organism Control can be a positive Control over how the organism(s) and target nucleic acid sequences in the test sample weathers the entire process. In yet some other aspects the quality control compositions of the invention, such as the Augmented Whole Organism Controls, are used in a concentration—or series of concentrations that may best simulate the potential concentrations of target organism/nucleic acid sequences that are suspected to be in the test sample which can act as a known benchmark for the results obtained from the sample to be screened/tested. Enhancing the number of features in the Controls to be tested against, whether it is number of organisms or target exogenous nucleic acid sequences, lowers the probability of false positives and negatives.

Further, in some other aspects of the invention, quality control compositions, or Augmented Whole Organism Control compositions, may further comprise isolated or protected nucleic acid sequences in known concentrations as both negative (off-target nucleic acids, for instance that are native to the organism (or strain or species of the organism to be tested for, without the target nucleic acid sequence) and positive (on-target nucleic acid sequences.

In some other aspects, the quality control compositions of the invention, such as the Augmented Whole Organism Controls, can be used to calibrate the NAT processes or run simultaneously through the NAT process as the sample to be tested.

In some other aspects, the quality control compositions, such as the Augmented Whole Organism Control compositions, of the invention are less pathogenic or non-pathogenic, by using whole organisms that have been rendered non-pathogenic by radiation, chemical exposure or the like and/or by using inherently less pathogenic or non-pathogenic organisms and/or non-pathogenic portions of the pathogenic causing target nucleic acid sequences an for which the sample to be tested is to be screened for.

In some other aspects, the quality control compositions of the invention, such as the Augmented Whole Organism Control materials, comprise two or more Whole Organisms wherein a first intact organism contains at least one nucleic acid sequence, tested in a NAT, that is a characteristic of that organism, and a second organism that contains a second nucleic acid sequence, tested in a NAT, that is not natively present in the first or second organism. In other aspects, the quality control compositions, such as the Augmented Whole Organism Control materials, of the invention comprise a Whole Organism combined with a free nucleic acid sequence wherein the whole organism contains at least one nucleic acid sequence that is a tested in a NAT and is characteristic of that organism, and the free nucleic acid sequence is tested in a NAT and comprises a characteristic not natively present in the first organism.

In some other aspects, the first intact vehicle or organism also comprises at least at least one nucleic acid sequence that is not natively present in the first or second vehicle or organism. In yet some other aspects, the second vehicle or organism comprises at least two nucleic acid sequences that are not natively present in the first or second vehicle or organism. In some aspects the vehicle is a protecting vehicle.

Provided herein is a composition comprising a Whole Organism Control wherein the Whole Organism Control comprises a whole organism containing (i) at least one nucleic acid sequence providing a characteristic of that organism that is tested in a Nucleic Acid Test (NAT), and (ii) a second nucleic acid sequence providing a characteristic not natively present in the organism that is tested in a NAT.

Also contemplated herein is a composition comprising a Whole Organism Control wherein the Whole Organism Control comprises a whole organism containing (i) at least a first nucleic acid sequence providing a characteristic not natively present in the organism tested in a Nucleic Acid Test (NAT), and (ii) a second nucleic acid sequence providing a second characteristic not natively present in the organism and is tested in a NAT.

In various embodiments, the disclosure provides a composition comprising a Whole Organism Control comprising two or more whole organisms wherein the composition comprises (i) a first intact organism containing at least one nucleic acid sequence that is a characteristic of that organism tested in a Nucleic Acid Test (NAT), and (ii) at least a second organism containing a second nucleic acid sequence providing a characteristic not natively present in the first or second organism and is tested in a NAT. In some other aspects either the first or second organism may comprise two or more nucleic acid sequences providing characteristic(s) not natively present in the first or second organism.

In various embodiments, the not natively present nucleic acid sequence is introduced into the whole organism by natural or by genetic modification methods. In certain embodiments, the not natively present nucleic acid sequence is introduced into the whole organism without integration into the genome sequences of the organism.

In various embodiments, the not natively present nucleic acid sequence is introduced into the protecting vehicle or organism of the quality control materials of the invention by integration into the genome of the organism. In certain embodiments, the not natively present nucleic acid sequence is integrated into the genome by an integrative vector or by Clustered Regularly Interspaced Short Palindromic Repeats (CRISPR), Transcription activator-like effector nucleases (TALEN) or Zinc-finger nucleases (ZFNs) technologies.

In various embodiments, the not natively present nucleic acid sequence is contained within a vector, introduced by transient transfection, introduced by viral transduction, or integrated into the genome of the protecting vehicle or organism.

In various embodiments, the not natively present nucleic acid sequence encodes all or part or a variant of a gene to be tested in a NAT.

It is contemplated that Nucleic Acid Tests (NAT) described herein comprise or utilize one or more of the following techniques: Polymerase Chain Reaction (PCR), Reverse-transcription Polymerase Chain Reaction (RT-PCR), multiplex PCR or RT-PCR, branched DNA assay, Ligase chain reaction, Transcription mediated amplification (TMA), Nucleic acid sequence based amplification (NASBA), strand displacement amplification (SDA), nucleic acid sequencing, and next-generation sequencing (NGS).

In various embodiments, the protecting vehicle can be selected from organisms (such as from the group consisting of bacteria, viruses, fungi, archaea, protists, parasites, and eukaryotic cells), or other protecting vehicles (such as a liposome). In other embodiments the first or second nucleic acid sequences of the invention could be in a plasmid or other vector within or, under certain conditions, outside a protecting vehicle or organism.

In various embodiments, when the Whole Organism Control comprises two or more organisms, the first and second organism may be the same species or different species within the organism family. In certain embodiments, when the Whole Organism Control comprises two or more organisms, the first and second or additional organisms may be of the same phylogenetic lineage or of different phylogenetic lineages.

In various embodiments, the first organism is a eukaryote and the second or additional organism is a eukaryote, bacterium, virus, fungi, archaea, protist, or parasite. In other embodiments as noted herein the first nucleic acid sequence can be in a plasmid, or liposome.

In various embodiments, the first organism is a bacterium and the second or additional organism is a eukaryote, bacterium, virus, fungi, archaea, protist, or parasite. In other embodiments as noted herein the first nucleic acid sequence can be in a plasmid, or liposome.

In certain embodiments, the first organism is a virus and the second or additional organism is a eukaryote, bacterium, virus, fungi, archaea, protist, or parasite. In other embodiments as noted herein the first nucleic acid sequence can be in a plasmid, or liposome.

In other embodiments, the first organism is a fungus and the second or additional organism is a eukaryote, bacterium, virus, fungus, archaeon, protist, or parasite. In other embodiments as noted herein the first nucleic acid sequence can be in a plasmid, or liposome.

In various embodiments, the first organism is an archaeon, and the second or additional organism is a eukaryote, bacterium, virus, fungi, archaea, protist, or parasite. In other embodiments as noted herein the first nucleic acid sequence can be in a plasmid, or liposome.

Also contemplated, the first organism is a protist, and the second or additional organism is a eukaryote, bacterium, virus, fungus, archaeon, protist, or parasite. In other embodiments as noted herein the first nucleic acid sequence can be in a plasmid, or liposome.

In various embodiments, the first organism is a parasite, and the second or additional organism is a eukaryote, bacterium, virus, fungus, archaeon, protist, or parasite. In other embodiments as noted herein the first nucleic acid sequence can be in a plasmid, or liposome.

In various embodiments, if the organism is a bacterium, the first organism and the second or additional organism is the same bacterial species or different bacterial species.

In various embodiments, if the organism is virus, the first organism and the second or additional organism is the same virus species or different virus species.

In various embodiments, the first organism is a eukaryotic cell and the second or additional organism is a bacterium.

Exemplary organisms contemplated for use in the methods are described in more detail in the Detailed Description.

In various embodiments, the disclosure provides a composition comprising a Whole Organism Control comprising a) a wild type first organism without any genetic modification, and a genetic sequence or sequences that are the target of a NAT; and b) a second organism that includes a modified nucleic acid sequence, that is also detected by a NAT.

In various embodiments, the disclosure provides a composition comprising a Whole Organism Control comprising a) a wild type first organism without any genetic modification, and a genetic sequence or sequences that are the target of a NAT; and b) a second organism that is also detected by a NAT.

In various embodiments, the second nucleic acid sequence encodes a gene selected from the group consisting of an antibiotic resistance gene, a drug resistance gene, a biomarker, an antigen, a virus gene, an oncogene, or a tumor suppressor gene. Exemplary antibiotic resistance genes and other genes contemplated for use in the methods are described in more detail in the Detailed Description.

In various embodiments, the first organism is Staphylococcus aureus and the second organism is Escherichia Coli, wherein the Escherichia Coli has been modified to contain the mecA gene.

In various embodiments, the organism is inactivated, optionally the inactivation is by formalin, aldehydes and other chemicals, gamma irradiation, UV irradiation, dehydration, X-ray, heat, or detergent. In some aspects, methods for inactivating pathogens for use as molecular controls using electromagnetic radiation, including, gamma irradiation, at a level that inactivates the biological activity of the control while leaving the sample DNA sufficiently intact for processing and analysis can be used.

Provided herein is a quality control composition, such as a composition comprising a Whole Organism Control, comprising (i) a whole organism containing at least one nucleic acid sequence providing a characteristic of that organism tested in a NAT, and (ii) a sufficient amount of free nucleic acid providing a characteristic not natively present in the organism and is tested in a NAT.

Also contemplated herein is a quality control composition, such as a composition comprising a Whole Organism Control, comprising (i) a whole organism containing at least one nucleic acid sequence providing a characteristic not natively present in that organism tested in a NAT, and (ii) a sufficient amount of free nucleic acid that is a second nucleic acid sequence providing a characteristic not natively present in the organism and is tested in a NAT.

Further contemplated is a method for detecting an organism or nucleic acid sequence in a biological test sample comprising: (a) obtaining a biological sample suspected of comprising the organism or nucleic acid sequence of interest; (b) providing a Whole Organism Control as described comprising an organism in a medium corresponding to a medium of the test sample; (c) detecting the organism or nucleic acid sequence of interest using Nucleic Acid Tests (NAT); and (d) comparing the levels of nucleic acids detected in the biological sample with the levels of nucleic acids detected in the Whole Organism Control.

The disclosure also provides a method for detecting an organism or nucleic acid sequence in a biological test sample comprising: (a) obtaining a biological sample suspected of comprising the organism or nucleic acid sequence of interest; (b) providing a multi-protecting vehicle or Multi-Organism Control comprising two or more whole protected nucleic acids or organisms in a medium corresponding to a medium of the test sample; (c) detecting the protecting vehicle, organism or nucleic acid sequence of interest using Nucleic Acid Tests (NAT); and (d) comparing the levels of nucleic acids detected in the biological sample with levels of nucleic acids detected in the Control.

In various embodiments, the medium for the test sample is selected from the group consisting of whole blood, serum, plasma, defibrinated plasma, stabilized plasma pool, cerebrospinal fluid, urine, saliva, semen, sputum, nasal swab, and vaginal swab.

Also provided herein is a kit comprising Whole Organism Controls as described herein, and instructions for use.

In various embodiments, the protecting vehicles in the quality control compositions or the Whole Organism Control comprises a nucleic acid sequence that encodes and expresses a protein(s) capable of detection in an immunoassay and wherein the protecting vehicle or Whole Organism Control can be used in both a NAT and an immunoassay. In related embodiments, the nucleic acid sequence encodes a gene selected from the group consisting of an antibiotic resistance gene, a drug resistance gene, a biomarker, a virus gene, an oncogene, or a tumor suppressor gene wherein the expressed protein is detectable in an immunoassay.

Also contemplated herein is a method for detecting an organism or nucleic acid sequence in a biological test sample comprising: (a) obtaining a biological sample suspected of comprising the organism or nucleic acid sequence of interest; (b) providing a quality control composition or Whole Organism Control as described herein comprising an organism in a medium corresponding to a medium of the test sample, and further comprising an expression element for a characteristic or protein not natively present in the Control; (c) detecting the organism or nucleic acid sequence of interest and/or the expressed element using Nucleic Acid Tests (NAT); (d) optionally detecting a protein encoded by the expressed element in an immunoassay or expression based assay; and (e) comparing the levels of nucleic acids and/or expressed element detected in the biological sample with levels of nucleic acids and/or expressed element detected in the Control.

In various embodiments, the protein(s) in the immunoassay contain at least one antibody epitope that is detected using an antibody.

In various embodiments, the quality control or Whole Organism Control is detected simultaneously by both a NAT and an immunoassay. In other embodiments, the quality or Whole Organism Control is detected sequentially by both a NAT and an immunoassay.

In various embodiments, the immunoassay includes an array, microarray formats, plate-based formats, bead-based formats, or gel-based formats.

In various embodiments, the Control for the immunoassay or expression-based assay is provided in a liquid or as a solid.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document. In addition, the invention includes, as an additional aspect, all embodiments of the invention narrower in scope in any way than the variations specifically mentioned above. With respect to aspects of the invention described or claimed with “a” or “an,” it should be understood that these terms mean “one or more” unless context unambiguously requires a more restricted meaning. With respect to elements described as one or more within a set, it should be understood that all combinations within the set are contemplated. If aspects of the invention are described as “comprising” a feature, embodiments also are contemplated “consisting of” or “consisting essentially of” the feature. Additional features and variations of the disclosure will be apparent to those skilled in the art from the entirety of this application, and all such features are intended as aspects of the disclosure.

### BRIEF DESCRIPTION OF THE DRAWINGS

The following figures are provided for illustrative purpose only of various aspects of the invention and are not meant to limit the scope of the invention as described herein.

FIG. 1A depicts a NAT in which a quality control composition, i.e. an Augmented Whole Organism Control is tested. In the figure, a NAT is used to detect a target nucleic acid sequence (e.g. mecA) in a sample containing MRSA (the test article), which is subject of a NAT, and a corresponding NAT Control which is a quality control composition used to assess the functionality of the NAT for the test article and/or to detect the target nucleic acid sequence in the test article. In the example, a Staphylococcus aureus acts as a protecting vehicle. It contains a non-natively present genetic material, e.g., a sequence derived from and characteristic of the target nucleic acid sequence, a gene mecA ( . . . ), which is detected in the NAT. The NAT comprises a component test “A” used to detect the presence of said genetic material, which NAT may be otherwise used to detect the presence of an organism that naturally contains similar genetic material, e.g., methicillin resistant Staphylococcus aureus (MRSA) (i.e. the test article). The method of the invention contemplates variations without limitation in which MRSA is any organism, and “A” is a component test of a NAT that may ordinarily be used to detect said organism. The variations include but are not limited to variations in which another modified cell or virus or other nucleic acid protecting vehicle takes the place of Staphylococcus aureus as a component of the NAT control.

FIG. 1B depicts a NAT for a target nucleic acid sequence in a whole organism (e.g. eukaryotic cell), wherein the that is a disease susceptibility or genetic marker which is subject of a NAT (test article) and a corresponding NAT Control which is a quality control composition used to assess the functionality of the NAT for the test article and to detect the target nucleic acid sequence in the test article. The figure illustrates a NAT in which an Augmented Whole Organism Control for the NAT comprises any whole organism as a protecting vehicle (e.g. eukaryote) with disease susceptibility or genetic marker containing the a non-natively present nucleic acid sequence that is tested in a NAT, including a component test (‘A’) to detect the disease susceptibility or genetic marker. The NAT control illustrates some embodiments of the invention wherein the non-natively present nucleic acid sequence is not integrated within the genome of the whole organism protecting vehicle.

FIG. 2A depicts a multitarget NAT and a quality control composition for same in which an Augmented Whole Organism Control is tested. In the example, the MRSA (the test article) comprises a nucleic acid sequence native to Staphylococcus aureus generally and a target nucleic acid sequence. The Augmented Whole Organism Control is an E. coli (an organism that differs from the organism in the test article, comprising two regions of non-natively present genetic material, e.g., a sequence from Staphylococcus aureus (native to the organism of the test article but non-native to the organism in the NAT Control) and a sequence from mecA (that is characteristic of the target nucleic acid sequence), is tested in a NAT, which includes a component test “A” used to detect said genetic material, which NAT may be otherwise used to detect the presence of an organism that naturally contains similar genetic material to both sequences, e.g. MRSA. The method of the invention contemplates variations without limitation in which MRSA is any organism, and “A” is a component test of a NAT that may ordinarily be used to detect said organism. The variations include but are not limited to variations in which another modified cell or virus takes the place of E. coli as a component of the NAT control. FIG. 2B depicts a NAT in which an Augmented Whole Organism Control comprises a Whole Organism Control (e.g. Eukaryote) with disease susceptibility or genetic marker containing both a non-natively present nucleic acid sequence and native nucleic acid sequence that are tested in a multitarget NAT, including a component test (‘A’) to detect the disease susceptibility or genetic marker.

FIG. 3 depicts a multitarget NAT in which the quality control composition is a multi protecting vehicle control, such as a Multi-Organism Control. In the figure, an Augmented Whole Organism Control, which is a Multi-Organism Control, is tested (and used to assess the functionality of the NAT for the test article, an MRSA which includes a nucleic acid sequence that is natively and general found in Staphylococcus aureus and a target nucleic acid sequence. In the example NAT Control, E. coli (a protecting vehicle for a second nucleic acid sequence) containing non-natively present genetic material, e.g., a sequence from mecA (a second nucleic acid sequence which is characteristic of the presence of the target nucleic acid sequence in the test article), and Staphylococcus aureus (a first protecting vehicle for a first nucleic acid sequence) comprising a natively-present sequence from Staphylococcus aureus (but not the target sequence), are tested in a NAT, which includes a component test “A” used to detect said genetic material, which NAT may be otherwise used to detect the presence of an organism that naturally contains similar genetic material to both sequences, e.g. MRSA. The method of the invention contemplates variations without limitation in which MRSA is any organism, and “A” is a component test of a NAT that may ordinarily be used to detect said organism. The variations include but are not limited to variations in which other modified cells or viruses take the place of E. coli and Staphylococcus aureus as components of the NAT control.

FIG. 4A depicts a multitarget NAT in which the quality control composition is a multi-protecting vehicle control (e.g. a Multi-Organism Control). The figure depicts an Augmented Whole Organism Control, which is a Multi-Organism Control, is tested in the NAT and used to assess the functionality of the NAT for the test article. In the example, Human adenovirus type 5 containing non-natively present genetic material, e.g., a sequence from and characteristic of mecA, mecA being a target sequence of the test article (collectively the adenovirus type 5 and the non-natively present genetic material is a second protected nucleic acid sequence, as that term is used herein), and Staphylococcus aureus comprising a natively-present sequence from Staphylococcus aureus (collectively a first protected nucleic acid sequence), are tested in a NAT, which includes a component test “A” used to detect said genetic material, which NAT may be otherwise used to detect the presence of an organism that naturally contains similar genetic material to both sequences, e.g. MRSA. The method of the invention contemplates variations without limitation in which MRSA is any organism, and “A” is a component test of a NAT that may ordinarily be used to detect said organism. The variations include but are not limited to variations in which other modified cells or viruses take the place of Staphylococcus aureus and Human adenovirus type 5 as components of the NAT control. FIG. 4B depicts a Multi-Organism Control: Augmented Whole Organism Control comprises a first organism (e.g. eukaryote) modified to contain a non-natively present nucleic acid sequence (collectively, a second protected nucleic acid sequence) and a second organism (e.g. bacterium) containing a natively-present nucleic acid sequence (collectively a first protected nucleic acid sequence), both of which are tested in a multitarget NAT to assess the functionality of the NAT for detecting the test article. In this example one of the organisms (or protecting vehicles) of the control is a bacterium and an organism in the test article is a bacterium. FIG. 4C depicts a Multi-Organism Control: Augmented Whole Organism Control comprising a first organism (e.g. eukaryote) modified to contain a non-natively present nucleic acid sequence and a second organism (e.g. eukaryote) containing a natively-present nucleic acid sequence, both of which are tested in a multitarget NAT. In these examples, the organisms of the NAT Control and the organisms of the Test Article are all eukaryotes, however, they are not necessarily the same eukaryotes. FIG. 4D depicts a Multi-Organism Control: Augmented Whole Organism Control for a test article depicted in the figure (an organism for example a eukaryote with a target sequence), the control composition comprising a first organism (e.g. virus) modified to contain a non-natively present nucleic acid sequence (a second protected nucleic acid sequence) and a second organism (e.g. eukaryote), containing a natively-present nucleic acid sequence (a first protected nucleic acid sequence), both of which are tested in a multitarget NAT used to assess the functionality of the NAT for the test article. FIG. 4E depicts a Multi-Organism Control: Augmented Whole Organism Control used to assess the functionality of the NAT for the test article comprising a first organism (e.g. bacterium) modified to contain a non-natively present nucleic acid sequence (a nucleic acid sequence characteristic of a target nucleic acid sequence in the test article) (collectively a second protected nucleic acid sequence) and a second organism (e.g. eukaryote) containing a natively-present nucleic acid sequence (a first protected nucleic acid sequence), both of which are tested in a multitarget NAT. FIG. 4F depicts a multi-protected nucleic acid sequence control, i.e., a Multi-Organism Control: Augmented Whole Organism Control for a multi organism “A” and “B” (and/or multi-target) test article comprising a first organism (e.g. bacterium) containing a native nucleic acid the presence of which is characteristic of Organism A, a second organism (e.g. virus) containing a native nucleic acid for NAT which is characteristic generally of the presence of organism “B” but not the target nucleic acid sequence of organism “B” (a first protected nucleic acid sequence), a third organism (e.g. virus) modified to contain a non-natively present nucleic acid characteristic of the target sequence of Organism “B” in the test article, each component being subject of a multiplex NAT and can be used to assess the functionality of the multiplex-NAT for the test article. FIG. 4G also depicts a Multi-Organism Control: Augmented Whole Organism Control used in a multiplex NAT for assessing the functionality of the multiplex NAT for a test article. The NAT Control depicted comprises a first organism (e.g. bacterium) containing a native nucleic acid, a second organism (e.g. virus) containing a native nucleic acid for NAT (a first protected nucleic acid sequence), a third organism (e.g. eukaryote), modified to contain a non-natively present nucleic acid for multiplex-NAT (a second protected nucleic acid sequence), all being tested in the NAT. FIG. 4H depicts a Multi-Organism Control: Augmented Whole Organism Control comprising a first organism (e.g. bacterium) modified to contain a non-natively present nucleic acid sequence (so a bacterium, or could be another organism or protecting vehicle is modified to comprise the sequence) the nucleic acid sequence characteristic of a target sequence in organism A, a second organism (e.g. virus) containing a native nucleic acid characteristic of the general organism B (but not the target sequence of organism B), for NAT, a third organism (e.g. eukaryote) modified to contain a non-natively present nucleic acid (i.e. target sequence of organism B) for testing in a multiplex-NAT and assessing the functionality of the multiplex-NAT for the Test Article.

FIG. 5A depicts a multitarget NAT in which an Augmented Whole Organism Control, which is a Whole-Organism Control admixed with a nucleic acid sequence, is tested. In the example, a plasmid containing genetic material, e.g., a sequence from and characteristic of mecA, (a second nucleic acid sequence, not protected) and Staphylococcus aureus comprising a natively-present sequence from Staphylococcus aureus (first protected nucleic acid sequence), are tested in a NAT, which includes a component test “A” used to detect said genetic material, which NAT may be otherwise used to detect the presence of an organism that naturally contains similar genetic material to both sequences, e.g. MRSA. The method of the invention contemplates variations without limitation in which MRSA is any organism, and “A” is a component test of a NAT that may ordinarily be used to detect said organism. The variations include but are not limited to variations in which other modified cells or viruses take the place of Staphylococcus aureus, and other forms or preparations of nucleic acid take the place of the plasmid as components of the NAT control. FIG. 5B depicts a Multi-Organism Control: Augmented Whole Organism Control comprising a first nucleic acid sequence, said first nucleic acid sequence in a vehicle (e.g. plasmid) containing a non-natively present nucleic acid sequence which is characteristic of a target sequence in the test article, and an organism (e.g. eukaryote) containing a natively present nucleic acid sequence, both of which tested in a multitarget NAT. FIG. 5C depicts a Multi-Organism Control: Augmented Whole Organism Control similar to FIG. 4G except for the third organism being replaced with a plasmid. The FIG. 5C depicts a Multi-Organism Control comprising a first vehicle (e.g. plasmid) contains a non-natively present nucleic acid sequence, and an organism (e.g. virus), containing a natively present nucleic acid sequence, and another organism (e.g. bacterium), containing a natively present nucleic acid sequence, all of which tested in a multiplex NAT.

FIG. 6A depicts a multitarget NAT similar to that depicted in FIG. 3 but in which the one of the protecting vehicles is a liposome. FIG. 6 depicts an Augmented Whole Organism Control, which is a Whole-Organism Control admixed with a liposome comprising a nucleic acid sequence, is tested. In the example, a liposome comprising genetic material, e.g., a sequence from mecA, and Staphylococcus aureus comprising a natively-present sequence from Staphylococcus aureus, are tested in a NAT, which includes a component test “A” used to detect said genetic material, which NAT may be otherwise used to detect the presence of an organism that naturally contains similar genetic material to both sequences, e.g. MRSA. The method of the invention contemplates variations without limitation in which MRSA is any organism, and “A” is a component test of a NAT that may ordinarily be used to detect said organism. The variations include but are not limited to variations in which other modified cells or viruses take the place of Staphylococcus aureus, and other forms or preparations of encapsulated nucleic acid take the place of the liposome as components of the NAT control. FIG. 6B depicts a Multi-Organism Control similar to FIGS. 4C, 4D and 4E where one of the protecting vehicles is a liposome. FIG. 6B depicts an Augmented Whole Organism Control comprising an encapsulated first organism (e.g. liposome) containing a non-natively present nucleic acid sequence, and a second organism (e.g. eukaryote) containing a natively present nucleic acid sequence, both of which tested in a multitarget NAT. FIG. 6C depicts a Multi-Organism Control similar to FIGS. 4F and 4G, where one of the protecting vehicles is a liposome. FIG. 6C depicts an Augmented Whole Organism Control comprising an encapsulated first organism (e.g. bacterium) containing a native nucleic acid, a second organism (e.g. virus) containing a native nucleic acid for NAT, and a third organism (e.g. liposome) containing a non-natively present nucleic acid for a multiplex-NAT.

FIG. 7: depicts a multitarget NAT quality control composition in which an Augmented Whole Organism Control, which is a Multi-Organism Control, is tested. In the example, Newcastle disease virus containing non-natively present genetic material, e.g., a sequence from PB2, and Influenza virus comprising a natively-present sequence, are tested in a NAT, which includes a component test “A” used to detect said genetic material, which NAT may be otherwise used to detect the presence of an organism that naturally contains similar genetic material to both sequences, e.g. hypervirulent influenza. The method of the invention contemplates variations without limitation in which hypervirulent influenza is any organism, and “A” is a component test of a NAT that may ordinarily be used to detect said organism. The variations include but are not limited to variations in which other modified cells or viruses take the place of influenza and Newcastle disease virus as components of the NAT control.

FIG. 8 depicts a quality control composition comprising an Augmented Whole Organism Control for an immunoassay and a NAT, Whole Organism Control comprises a whole organism expressing at least one nucleic acid sequence providing a characteristic of that organism tested in a NAT including a component test (‘A’), and one antibody epitope tested in an immunoassay, for use in NAT and immunoassay, wherein the Whole Organism Control is formulated such that it is suitable for use in either test.

FIG. 9 depicts a quality control test and composition comprising an Augmented Whole Organism Control for immunoassay and a NAT, the Whole Organism Control comprising a whole organism containing at least one nucleic acid sequence providing a characteristic of that organism tested in a NAT, and additionally containing (or a second organism containing) an antibody epitope providing a characteristic of that organism tested in an immunoassay, wherein the Whole Organism Control is formulated such that it is suitable for use in either test.

FIG. 10 depicts a generalized example of the method of the invention in a Multiplex NAT comprising a component test “A” which is a NAT that is usually used to detect a natively present sequence from an organism “A”, and a component test “B”, which is a multiplex NAT that is usually used to detect two natively-present sequences from an organism “B”. In the example, the Augmented Whole Organism Control is comprised of three organisms, one of which comprises a sequence detected in component test “A”, and two other organisms together comprising the sequences detected in component test “B”, all three sequences being tested in the multiplex NAT. The method of the invention contemplates variations without limitation in which A is any organism, and “A” is a component test of a NAT that may ordinarily be used to detect said organism, and in which B is any organism, and “B” is a component test of a NAT that may ordinarily be used to detect said organism, and in which additional component tests and corresponding organisms may be tested, without limitation. The variations include but are not limited to variations in which other combinations or modified cells or viruses or free or encapsidated nucleic acids take the place of those shown as components of the NAT control.

### DETAILED DESCRIPTION OF THE INVENTION

Disclosed herein are compositions for use as quality controls for assessing the functionality of a Nucleic Acid Test (NAT). Also disclosed are related methods for preparing and using same and kits.

NATs operate by detecting particular nucleic acid sequences of interest (target nucleic acid sequence) in a test article (such as, an organism) in a sample (such as a biological sample, such as a human, veterinary, or environmental (e.g., air, water, or soil) sample).

As an example, to detect the antibiotic resistant bacterium Methicillin-Resistant Staphylococcus aureus (MRSA), a test article, from a nose or wound swab sample (sample) of a patient, the target nucleic acid sequence would in some embodiments be mecA which is the gene identified as conferring antibiotic resistance to the Staphylococcus aureus (e.g. that results in MRSA). In some aspects NATs are used to provide evidence of the presence of said test articles (e.g. organisms such as MRSA).

The basic features of a NAT in one example include: a sample (or test article) processing step, nucleic acid extraction steps (including in some embodiments lysis and isolation), and often, an amplification step and a detection step.

So, in developing quality control compositions and materials for assessing the functionality of a NAT for detecting a specific target nucleic acid sequence or test article (e.g. organism), there is a need to assess the ability of nucleic acid sequences to survive the NAT through to the detection step. For instance, in some aspects of the invention, the controls presented herewith may be used in a method to assess the effect of the NAT on the degradation or loss of nucleic acid sequence though the process. In some other aspects of the invention, the controls presented herewith may be used to better assess whether a negative result actually means a negative result or was the result of nucleic acid sequence degradation through the process or other reasons. In yet some other aspects of the invention, the controls presented herewith may be used to better assess whether a positive result actually means a positive result or was the result of detection methods (e.g. primers in a PCR amplification or probes or selected target sequences) are not sufficiently specific, or due to contamination of any of the components, or for other reasons. Similarly, for other components of the NAT, regarding ability of the process used to extract and, in some embodiments, to amplify and detect a nucleic acid sequence(s), to assess the presence of any inhibitors, the quality of reagents, and/or the proper functioning of equipment.

Further, in developing or selecting features of a quality control composition for a NAT, in some aspects one selects for features that would best simulate the processing of a sample or test article through the multiple NAT steps. The use of a control comprising the organism which is the target of the NAT is often not desirable, due to the pathogenicity, virulence or toxicity of the organism, handling issues, growth or culture challenges to produce the control. As such, it is desirable to develop a system for creating quality control compositions that does not comprise the entire native test article or at least not the-pathogenic, virulent or toxic form of a test article, but yet is characteristic of the presence of the test article.

In another aspect, as different testing centres may use different NATs that select for different characteristic features of a test article including selecting for different nucleic acid sequences for detection for the same test article, in one aspect it is desirable to develop quality control compositions that could be used to assess the functionality of more than one such NAT for the test article.

In carrying this point further, it is possible to construct NAT to recognize, for example, the presence of a particular species of an organism, by addressing the presence of nucleic acid sequences (‘target sequences’), and in most organisms a very diverse choice of possible target sequences exists. Many controls are manufactured that are useful only for a particular NAT or a particular set of NAT, but not for all NAT. Such controls, for instance, may contain only a portion of the nucleic acid of the material that is to be tested, for example sequences encompassing the target sequences, but not the entire sequence of the material to be tested. In some aspects, the present invention provides for the preparation of a control that is broadly useful in NAT for a particular organism. In some aspects of the invention, all or most of the many possible target sequences are present in the control composition in one or more vehicles. One method of constructing such a control is to obtain a typical example of the organism, and present it in an appropriate quantity and an appropriate sample matrix for use as a control. However, obtaining such organisms to provide controls can present problems for the manufacturer of Controls. These problems include difficulties in obtaining said organisms for a multitude of reasons (transportation prohibitions, remote location of specimens, the hazardous nature of some organisms leading to importation barriers, and the unwillingness of those in possession of such organisms to provide them, and other reasons). Further, to manufacture Controls in sufficient quantity for general distribution, it is sometimes necessary to propagate or grow or culture such organisms. Some organisms may be biohazardous and may require significant and costly facilities, equipment, personnel, and supplies to grow the organisms as a consequence, which can slow or render impractical the manufacture of controls using such organisms. Further, to manufacture controls that are responsive to rapidly-evolving organisms including pathogens, it is necessary to provide controls that include new genetic sequences that represent new strains of organisms, which can be costly and time-consuming and can delay the availability of such controls for the reasons stated above. In some aspects, the present invention addresses and provides for one or more of these issues.

Further, in some aspects of the invention, more than one nucleic acid sequence is detected in a NAT, either to detect multiple regions of a nucleic acid sequence of a target nucleic acid sequence or multiple target nucleic acid sequences or to detect off-target characteristic features or nucleic acid sequences that are characteristic of a strain, specifies or family of an organism in the test article that does not have the target nucleic acid sequence. In some embodiments the detection of only one nucleic acid sequence in a NAT is required to determine the presence of the target nucleic acid sequence in the test article, in other embodiments, the detection of more than one nucleic acid sequence is required to determine the presence of the target nucleic acid sequence and/or the test article. In another aspect, the quality control compositions comprise multiple nucleic acid regions of the target nucleic acid sequence, to enable it to be detected in more than one or a multitude of NATs for the target nucleic acid sequence. Similarly, for controlling for the detection of off-target nucleic acid sequences that may in some embodiments be specific to the test organism without the target nucleic acid sequence. Thus, the inventors have developed quality control compositions that can be more universal quality control compositions.

The present invention provides a system and method for developing quality control materials and compositions for NAT and quality control materials and compositions that can be used alone or in combination with other NATs or other detection tools, such as immunoassays for detecting the same or different test articles in a test sample. The present invention further provides kits comprising the quality control compositions and/or components of same and/or for making any of the foregoing and instructions for so doing and for using said compositions and components.

In some aspects, the quality control compositions used to assess the functionality of the NAT provided for in the present invention is an Augmented Whole Organism Control comprising materials used in a Nucleic Acid Tests (NAT) for assessing the functionality of the NAT and assessing methods for detecting an organism in a biological test sample.

The present disclosure contemplates the use of quality control compositions, such as Augmented Whole Organism Control materials for Nucleic Acid Tests (NAT). In various embodiments, the quality control compositions of the invention useful to assess the functionality of a NAT, wherein the NAT is used to detect the presence of an organism and a target nucleic acid sequence in a test article, comprise: (i) one, or in some embodiments, more than one, first protected nucleic acid sequence comprising a first nucleic acid sequence the presence of which is characteristic of the presence of the organism in the test article said first nucleic acid sequence being tested in the NAT; and (ii) one, or in some embodiments more than one, second protected nucleic acid sequence comprising a second nucleic acid sequence the presence of which is characteristic of the presence of the target nucleic acid sequence in the test article said second nucleic acid sequence being tested for in the NAT, wherein the first and second protected nucleic acid sequences are together in one or separately in more than one protecting vehicle, wherein the protecting vehicles can be the same or different type of protecting vehicle, wherein the second protected nucleic acid sequence is not natively present in its protecting vehicle.

In some embodiments, at least one of the protecting vehicles (such as in some embodiments, an organism) has the same or similar nucleic acid protection properties when processed through the NAT as the organism the presence of which is being tested for in the test sample.

In various embodiments, the protecting vehicle of the first nucleic acid sequence (such as in some embodiments, an organism) that has the same or similar nucleic acid sequence protection properties when processed through the NAT as the organism or organism comprising the target sequence the presence of which is to be tested for in the test article, wherein the protecting vehicle does not comprise the target nucleic acid sequence.

In various embodiments, the quality control composition of the invention: (i) the organism comprising the target nucleic acid sequence to be tested for in the NAT is a virulent or pathogenic or toxic organism or wherein when the target nucleic acid sequence is present with or without additional sequences a virulent or pathogenic or toxic trait is conferred on the organism; and (ii) the protecting vehicle of the first protected nucleic acid sequence is a less or non-virulent and/or pathogenic form of the organism comprising a native genomic nucleic acid sequence that does not comprise the target nucleic acid sequence.

In various other embodiments, the second nucleic acid sequence is: (i) selected from a portion of the nucleic acid sequence characteristic of the target nucleic acid sequence, or (ii) is modified, such that the second nucleic acid sequence would not be expressed to confer the expression of the genetic feature of the target sequence, either alone or in combination with other nucleic acid sequences in the composition.

In some embodiments the quality control compositions of the invention comprises more than one first nucleic acid sequence, and each first nucleic acid sequence being a different nucleic acid sequence characteristic of the organism in the test article, at least one but not necessarily all, of the first nucleic acid sequences being tested for in a NAT.

In yet other embodiments, the quality control composition of the invention is a Whole Organism Control comprising; (a) a less virulent or less pathogenic or less toxic version of the organism for which the sample is to be screened, which is less toxic or less pathogenic or less virulent or dangerous than the organism to be screened for in the NAT; and (b) one or more nucleotide sequences that are determinative of the presence of the virulent, pathogenic or toxic strain of a whole organism containing (i) at least one nucleic acid sequence providing a characteristic of that organism that is tested in a Nucleic Acid Test (NAT), and (ii) a second nucleic acid sequence providing a characteristic not natively present in the organism that is tested in a NAT.

In some other aspects of the invention, the quality control compositions are Augmented Whole Organism Control composition/materials, wherein the Augmented Whole Organism Control comprises a whole organism comprising at least one nucleic acid sequence providing a characteristic of that organism tested in a NAT, and comprising a second nucleic acid sequence providing a characteristic not natively present in the organism and is tested in a NAT. In some other aspects, the Augmented Whole Organism Control materials comprise two or more whole organisms wherein the Control materials comprise a first intact organism comprising at least one nucleic acid sequence that is a characteristic of that organism tested in a NAT, and a second organism comprising a second nucleic acid sequence providing a characteristic not natively present in the first or second organism and is tested in a NAT. For instances where there exist naturally-occurring organisms that comprise both the first and second nucleic acid sequences, these organisms are not to be confused with the Augmented Whole Organism Controls for use herein.

### Definitions

The plural herein shall equally denote the singular, and the singular shall equally denote the plural wherever reasonable. The words “for example” or “by way of example” or similar phrases shall be interpreted as equivalent to “for example but not by way of limitation”, such that any example shall not limit the generality to which the example pertains.

### Comprise

The present invention can comprise or consist essentially of the components of the present invention as well as other ingredients or elements described herein and/or ingredients or elements not described. As used herein, “comprising” or “comprise” or “comprised” or “comprises”, or “comprised of” or other variants of the term, mean the elements recited, or their equivalent in structure or function, plus any other element or elements which are not recited. Further, the terms should be considered synonymous with equivalent variants of ‘including’, ‘containing’, or ‘characterized by’, interpreted in the broadest and most inclusive way consistent with the context, and are open ended and do not exclude additional unrecited elements or method steps. The terms “having” and “including,” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention.

### “Characteristic Of”

“Characteristic of” is used herein in reference to a feature or property (such as a nucleic acid sequence, or protein, or epitope), that is sufficiently specific to a feature or property of interest (such as a target nucleic acid sequence or organism) to enable it to be used to indicate or provide evidence of or associate it with the presence of said feature of interest (e.g., said target nucleic acid sequence or organism). The term is often used here as “characteristic of a target nucleic acid sequence” or “characteristic of” an organism or test article or “characteristic of” the presence of any of the foregoing.

### Nucleic Acid Tests (NAT)

As used herein, “nucleic acid tests” or “NAT” or “NATs” refer to one or more tests for the presence or absence of a biological organism or trait or genetic element that may be performed utilizing methods that detect the nucleic acids, or nucleic acid sequences, of the organism or the organism trait or element. For example, NAT may be based on the detection of DNA or of RNA sequences of the organism or trait or genetic element. NAT may use known methods for detection of nucleic acids, including, but not limited to, Polymerase Chain Reaction (PCR), Reverse-transcription Polymerase Chain Reaction (RT-PCR), branched DNA assay, ligase chain reaction, transcription mediated amplification (TMA), Nucleic acid sequence based amplification (NASBA), strand displacement amplification (SDA), nucleic acid sequencing, next-generation sequencing (NGS), loop-mediated isothermal amplification, helicase-dependent amplification, cycling probe technology, ligase chain reaction, invader technology, and hybrid capture. In addition, new detection methods may be developed that generally rely on the presence and/or sequence of nucleic acids, to which the present invention is also applicable and pertinent, and which are contemplated as NAT as defined and included herein.

Such tests for the presence and/or quantitation of organisms may function by the detection of a large or complete portion of the genome of an organism of interest (for example, by restriction enzyme digestion and gel electrophoresis, or by sequencing), or may function by the detection of a smaller portion of the genome of the organism of interest (for example, PCR-mediated amplification of a portion of the genome based on the ability of exogenously added nucleic acid oligomers to bind to a complementary portion of the organism's genome and to prime the polymerase-mediated amplification of such a portion).

NAT may be carried out for any of a variety of reasons, including but not limited to infectious disease diagnostics, infectious disease surveillance, genetic disease diagnostics, genetic disease surveillance, environmental science, forensics, research, development, archeology, food safety, animal health, and addressing many other scientific and practical questions. Such testing may be qualitative or quantitative or both. In some other aspects, NAT can be used to detect good, desirable or beneficial organisms or the absence of bad, undesirable, not-beneficial organisms, for instance desirable and undesirable gut flora, or environmental water flora.

Such testing may be performed with high or low sensitivity or specificity to suit different requirements and applications.

### Immunoassay

As used herein, “Immunoassay” refers to any test for the presence or absence of a biological organism or trait or element that may be performed utilizing methods of detection that comprise affinity and/or complementarity-mediated binding of entities, including but not limited to antibodies, immunoglobulins, DNA oligomers, aptamers, protein scaffolds, protein receptors, or other binding moieties, to elements or traits of the organism or the trait. For example, immunoassays may be based on the detection of specific polypeptide or carbohydrate sequences of the organism or trait. “Immunoassay” may also refer to one or more tests and may use known methods for detection, including, but not limited to, ELISA, Immunoprecipitation, blotting assays, Surface Plasmon Resonance, and many others. In addition, new detection methods may be developed that generally rely on the presence of binding species, to which the present invention is also applicable and pertinent, and which are contemplated as immunoassays as defined and included herein.

Such tests for the presence and/or quantitation of organisms may function by the detection of a large or complete portion of the element of an organism of interest, or may function by the detection of a smaller portion of the element of the organism of interest (for example, a peptide fragment).

Immunoassays may be carried out for any of a variety of reasons, including but not limited to, infectious disease diagnostics, infectious disease surveillance, genetic disease diagnostics, genetic disease surveillance, environmental science, forensics, research, development, archeology, food safety, animal health, and addressing many other scientific and practical questions. Such testing may be qualitative or quantitative or both. Such testing may be performed with high or low sensitivity or specificity to suit different requirements and applications.

### Expression-Based Assay

As used herein, “Expression-based assays” refer to any test for the presence or absence of a biological organism or trait or element that may be performed utilizing methods of detection that use binding of an externally provided element to elements of the organism or the organism trait. Such assays may be performed in combination with or by means of immunoassay methods or NAT. For example, DNA aptamers may be used for the detection of specific polypeptide or carbohydrate moieties of the organism or trait. “Expression-based assays” may also refer to one or more tests, including, but not limited to any of the methods described or contemplated as ‘NAT’ or ‘immunoassay’ herein, and which may include formats such as array or microarray formats, plate-based formats, bead-based formats, gel-based formats, and many other formats known to those in the art. In addition, new detection methods and formats may be developed that generally rely on the similar principles or modes of operation, to which the present invention is also applicable.

Such tests for the presence and/or quantitation of organisms may function by the detection of a large or complete portion of the element of an organism of interest, or may function by the detection of a smaller portion of the element of the organism of interest (for example, a peptide fragment).

Expression-based assays may be carried out for any of a variety of reasons, including but not limited to infectious disease diagnostics, infectious disease surveillance, genetic disease diagnostics, genetic disease surveillance, environmental science, forensics, research, development, archeology, food safety, animal health, and addressing many other scientific and practical questions. Such testing may be qualitative or quantitative or both. Such testing may be performed with high or low sensitivity or specificity to suit different requirements and applications.

### Multitarget NAT

As used herein, “multitarget NAT” refers to a NAT that simultaneously detects or measures more than one sequence within an organism to determine some coordinated or correlative property of interest (i.e. ‘a genetic trait’ or ‘characteristic’, these terms are used interchangeably herein). A multitarget NAT may be comprised of NAT performed simultaneously or sequentially and can be performed in the same reaction environment or different reaction environments. For example, but not by way of limitation, a multitarget NAT testing for the presence of methicillin resistant Staphylococcus aureus may be comprised of a NAT testing for a first sequence characteristic of the presence of Staphylococcus aureus, and a second NAT, performed simultaneously or sequentially, testing for a sequence characteristic of methicillin resistance. All assays disclosed herein, including immunoassays, may be multitarget.

### Multiplex NAT

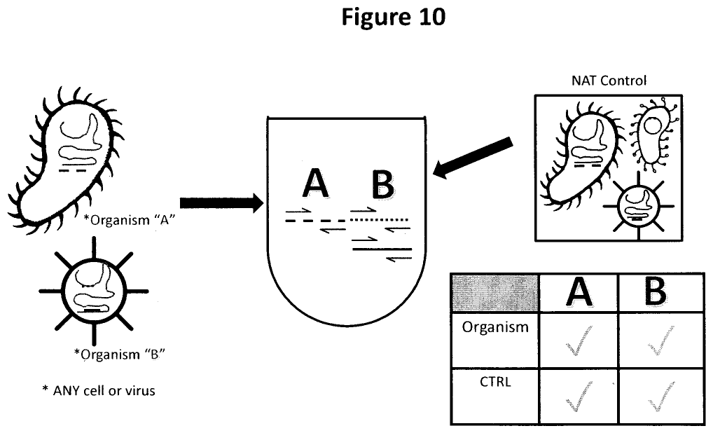

As used herein, “multiplex NAT” refers to a test, comprising two or more NAT and/or multitarget NAT that simultaneously detects or measures two or more organisms and/or two or more distinct properties in a test article or biological test sample. All assays disclosed herein, including immunoassays, and all controls therefore, should be considered to be operable as multiplex assays or controls for multiplex assays, whether explicitly so described or not, unless such is clearly incompatible with the context. FIG. 10 illustrates certain embodiments of a generalized Whole Organism Control for a Multiplex NAT of the invention.

### Sample and Biological Test Sample

As used herein, a “sample” or “biological test sample”, or “test sample”, refers to a sample to be tested in the NAT and are often used interchangeably herein. In some aspects it also includes or refers to the “test article” which can be derived from a sample (e.g. through processing of the sample). A “test article” as used herein refers to one or more articles (such as an organism) suspected of being in the sample and which is subject to and tested for in a NAT, In various embodiments, a biological test sample is compared to the quality control composition of the invention, such as the Augmented Whole Organism Control Materials to detect an organism or nucleic acid of interest. In some embodiments, the operation of the assay will be ascertained (or confirmed or validated or challenged) using the quality control composition of the invention to support an independent or coincident detection of an organism or nucleic acid or other element of interest within a sample. The “medium of the test sample” or of “the sample” or of “the biological sample” refers to the fluid or other medium from which the sample is derived or in which the sample is stored. Further, a medium may correspond to a biological medium or other medium and include but is not limited to, phosphate buffered saline (PBS), other organic or inorganic buffers, and engineered fluids that may mimic some property or properties of blood or blood components or other bodily fluids, or other mediums contemplated herein. Further, sometimes the fluid of the sample is the transport medium, which may or may not include organic components. Exemplary sources or mediums of test sample include whole blood, serum, plasma, defibrinated plasma, stabilized plasma pool, cerebrospinal fluid, urine, saliva, semen, sputum, nasal swab, and vaginal swab.

### Organism

As used herein, an “organism” refers to a cell or cells of a living being, including viruses, where consistent with the context. Organism includes a eukaryote, eukaryotic cell, prokaryote, prokaryotic cell, bacterium, virus, fungus, fungal cell, archaeon, protist, or parasite. For example, and by way of clarification and not limitation, the “organism” in the case of human cells refers to the cells. By way of further example and clarification, but not limitation, cells bearing a genetic mutation or difference within a population of cells not bearing that mutation or difference may be considered an organism distinct from the cells not bearing said mutation or difference. As used herein, “organism family” refers to all members of the phylogenetic tree. As used herein, “organism” shall be construed to refer broadly to the organism family when consistent with the context, and more narrowly to a subset of strains of the organism family in the context of particular genetic traits that may not be shared widely or commonly by members of an organism family.

### Whole Organism

As used herein, a “Whole Organism” refers to an organism, viable or nonviable, comprised of intact or largely intact cells or viruses from fractions or portions or preparations of whole organisms. Whole organisms may be optionally purified, retaining at least some but not necessarily all of the gross structure of the cell or virus.

### Vehicle

As used herein a vehicle is any nucleic acid comprising entity or carrier that may encapsidate, encapsulate or comprise a nucleic acid, it can be an organism, such as a whole organism but is not necessarily a live organism, in some aspects, it includes, but is not necessary limited to a liposome (e.g., from 0.1 nm-3 μm), microsphere (e.g., from 0.1-100 μm), episome, plasmid (e.g., from 1-200 kbp) and other largely intact synthetic body that comprises or contains one or more nucleic acid sequences for use in a NAT or other assay. It further includes but is not limited to free nucleic acid, terminally protected nucleic acid, plasmid, phage, cosmid, a protecting vehicle and an organism.

### Protecting Vehicle

A protecting vehicle is any enclosing or associating substance or barrier, including but not limited to: a bacterium, eukaryotic cell, protein capsid or shell (such as, viral capsids, a protein capsid or shell), microcompartments, engineered protein shells, virus, a virus-like particle, liposome, an anucleated cell, a vesicle, a nanovesicle, an exosome, a micelle, a solid lipid nanoparticle, a lipid-coated particle, a nanotube, a nanocrystal, a polymeric nanoparticle, an inorganic nanoparticle, interpolyelectrolyte complex, (polyplex) or a dendrimer, armored RNA, or other suitable physical boundary to inhibit the loss or degradation of nucleic acid sequence in the NAT up to the nucleic acid extraction process step of the NAT and/or to enable recovery (preferably substantial recovery) of the nucleic acid sequence in the nucleic acid extractions steps of the NAT and/or that sufficiently protects the nucleic acid sequence within it (such as the first and second nucleic acid sequence, as those terms are used herein) to enable its detection in the NAT. “Protection properties” has a similar and corresponding meaning referring to the extent a vehicle can protect the nucleic acid sequence within or associated with it based on any one or more of the foregoing features (i.e. to inhibit the loss or degradation of nucleic acid sequence in the NAT up to the nucleic acid extraction process step of the NAT, and/or to enable recovery (preferably substantial recovery) of the nucleic acid sequence in the nucleic acid extractions steps of the NAT and/or that sufficiently protects the nucleic acid sequence within it (such as the first and second nucleic acid sequence, as those terms are used herein) to enable its detection in the NAT). In some aspects, the protection of the nucleic acid sequences may be mediated by their containment within or their association with the protecting vehicle.

In some aspects, the protecting vehicle such as one or more of the first and one or more of the second protecting vehicles can all be independently selected from the group comprising or in some other embodiments consisting of: a virus, a virus-like particle, a bacterium, a eukaryotic cell, an anucleated cell, a liposome, a vesicle, a protein capsid or shell, a microcompartment, a nanovesicle, an exosome, a micelle, a solid lipid nanoparticle, a lipid-coated particle, a nanotube, a nanocrystal, a polymeric nanoparticle, an inorganic nanoparticle, an interpolyelectrolyte complex (polyplex), or a dendrimer.

### Substantially Recovered in the Nucleic Acid Extraction Steps of the NAT

Substantially recovered in the nucleic acid extraction steps of the NAT refers to recovery of the nucleic acid extraction steps of the NAT sufficient to enable detection of same in the nucleic acid detection step of the NAT, and in a preferred aspect of the invention is 100% or great than 90%, or greater than 80% or greater than 70% or greater than 60% or greater than 50% or greater than 40% or greater than 30% or greater than 20% of the nucleic acid that was present in the original sample or test article as the case may be, or for greater certainty to any quantity that is recovered in the nucleic acid extraction steps that is sufficient for the proper functioning of the NAT according to its design intention and/or to the requirements of the user(s) of the NAT.

### Intact

As used herein, “intact” refers to the gross structure of a cell or virus, retaining at least some but not necessarily all of the relationships between its usual component parts. For example, intact gross structure includes spatial relationships between its native component parts including the presence of nucleic acids within some complete or partial residue or remainder of the structure of the cell or virus, even if incomplete, as it exists in its living or viable state.

### Inactivation

As used herein, “inactivation” of organisms refers to treatment of organism resulting in a significant defect or a lack of reproductive or infectious capacity. Methods of inactivation include, but are not limited to, electromagnetic irradiation, infrared irradiation, visible light irradiation, laser irradiation, irradiation by a lamp, bulb, tube, filament, antenna, light-emitting diode, particle accelerator or other device, irradiation produced by radioisotopes, radiation from electrical or electronic devices, natural irradiation, artificial irradiation, ultraviolet irradiation, alpha irradiation, beta irradiation, gamma irradiation, or X-ray irradiation, solvent treatment, detergent treatment, pasteurization, acidic pH inactivation, basic pH inactivation, covalent chemical modification of nucleic acids, covalent chemical modification of proteins, noncovalent chemical modification of nucleic acids, noncovalent chemical modification of proteins, chaotropic disruption, treatment with formalin, formaldehyde, paraformaldehyde, glutaraldehyde, ortho-phthalaldehyde, other aldehydes, beta propiolactone, hydrogen peroxide, other peroxides, chlorine and chlorine compounds, ammonia and ammonium compounds, amines, amine-reactive compounds, carboxylic compounds and carboxyl-reactive compounds, peracetic acid, phenolic compounds, and other chemically or physically reactive or disruptive compounds, physical disruption, freeze-thaw disruption, heat inactivation, pasteurization, other temperature-based and/or time based inactivation, and other means. In some aspects, methods for inactivating pathogens for use as molecular controls using electromagnetic radiation, including, gamma irradiation, at a level that inactivates the biological activity of the control while leaving the sample DNA sufficiently intact for processing and analysis, can be used.

### Control

As used herein, a “Control” or “Controls” may refer to one or more samples in which the presence or absence of an analyte is known, that may be used in an assay, test process or test procedure (‘test’) that results in a measurement or determination of some property, whether qualitative or quantitative. Controls may be used to design or develop a test, to establish proper test function, to challenge or confirm or test the operation, performance, equipment, systems or procedures associated with the test, to provide evidence of the proper functioning and/or the validity of the results of the test. In some cases, but not all, the use of controls may be required by regulation, law, or principles of best practice. Controls may be extrinsic to the test articles of interest or may be tested independently of the test articles of interest, and are referred to herein as “External Controls” or “Extrinsic Controls”. Alternatively, the Controls may be integrated with or mixed with the test article of interest, in which case they are referred to herein as “Internal Controls”.

### Whole Organism Control

As used herein, “Whole Organism Control” refers to a control comprising one or more whole organisms as described herein. “Whole Organism Control” is also used interchangeably with “Whole Organism Control materials”.

### Multi-Organism Control

As used herein, a “Multi-Organism Control” refers to type of a quality control compositions for a NAT, such as a Whole Organism Control comprising two or more viable or nonviable or inactivated whole organisms, including fractions or portions of whole organisms, whether purified or not, or from synthetic or biosynthetic materials.

### Molecular Control

As used herein “Molecular Control” refers to a control used in or useful for a NAT or other assay described herein. Molecular controls may comprise, but are not limited in composition to, nucleic acid, water, solvent, or buffer for use in an assay.

### Whole Organism Molecular Control

As used herein, “Whole Organism Molecular Control” refers to a molecular control comprising one or more whole organisms.

### Augmented Whole Organism Molecular Control,

As used herein, is a type of quality control composition used to assess the functionality of a NAT. “Augmented Whole Organism Molecular Control”, “Augmented Whole Organism Control” and “Augmented Molecular Control” refer to a Whole Organism Molecular Control comprising at least one whole organism and one or more non-natively present, nucleic acid sequence (which may be constructed from naturally-occurring sequences), and denote the singular or plural, construed in the most inclusive way consistent with the context. In certain embodiments, the augmented whole organism control may contain the exogenous nucleic acid within the genome of the organism. In other embodiments, the augmented control may comprise the nucleic acid in a vehicle, e.g., plasmid, microsphere, liposome, or episome, or separate organism for carrying nucleic acid within a cell. In yet other embodiments, the nucleic acid may be external to the whole organism.

### Genetically Modified Organisms (GMOs)

As used herein, “genetically modified organisms (GMOs)” refer to organisms that have been modified by genetic modification methods. GMOs are, by virtue of their modification by genetic modification methods, not naturally-occurring organisms, and they include nucleic acid sequences not natively present in the organism(s) from which they derive.

### Genetic Modification Methods

As used herein, “genetic modification methods” refers to modification of an organism to introduce one or more nucleic acids or sequences of nucleic acids not natively present in the organism (i.e., introduce an exogenous nucleic acid not endogenous to the organism before the application of the genetic modification method). For clarity, genetic modification methods comprise methods wherein a first organism or type of organism is modified, thereby producing a second organism wherein the nucleic acid content of the second organism is not identical to the nucleic acid content of the first. As used herein, the phrase “not naturally occurring” or “not native”, and variants thereof, refers to nucleic acids or nucleic acid sequences not present in a first organism that may be introduced by a genetic modification method to create a second organism. The nucleic acids introduced may optionally encode all or part of a gene, or product thereof, to be tested. Genetic modification methods, i.e., modification of an organism, introduce one or more nucleic acids not natively present in the organism. Nucleic acids may be introduced into an organism using one or more of the many methods that are known to those skilled in the art, including but not limited to transient transfection (e.g. lipid or lipid nanoparticle transfection), viral transduction (e.g. using retroviral, adenoviral/adeno-associated viral vector [AAV], herpes simplex virus or lentivirus vector systems), mutation and selection, directed evolution, selection and propagation of organisms, and/or any methods resulting in integration into the genome of the organism. Methods resulting in integration into the genome of the organism may be targeted (i.e. directed to a chosen region or position within the host genome), or untargeted (i.e. within a region not specifically chosen or controlled, as for example in ‘random integration’). In some related aspects, nucleic acid sequences may also be introduced into the whole organism without integration into the genome of the organism. It should be readily understood that genetic modification methods exist in a large variety of forms, and that methods continue to be developed and/or modified, all with a common goal of effecting the creation of or a change in the nucleic acid content of an organism, or to create a second organism derived from a first organism by change in the nucleic acid content of the organism. The present invention is applicable to organisms derived through any genetic modification methods presently known and to those methods that will be developed to effect similar results.

Methods of introducing nucleic acids by viral transduction, or transient or non-transient transfection are well-known in the field of molecular biology.

In various embodiments, one or more non-native or exogenous nucleic acids may be integrated into the genome of the organism using an integrative vector or by one or more gene editing technologies, including but not limited to the following: Clustered Regularly Interspaced Short Palindromic Repeats/CRISPR associated (CRISPR/Cas) systems (e.g. CRISPR/Cas9), Transcription activator-like effector nucleases (TALEN) or Zinc-finger nucleases (ZFNs) technologies.

### Zinc-Finger Nucleases (ZFNs) and Transcription Activator-Like Effector Nucleases (TALENs)

Zinc-finger nucleases (ZFNs) and Transcription activator-like effector nucleases (TALENs) are customizable DNA-binding proteins that comprise DNA-modifying enzymes. Both can be designed and targeted to specific sequences in a variety of organisms (Esvelt and Wang, Mol Syst Biol. (2013) 9: 641, which is incorporated by reference in its entirety). ZFNs and TALENs can be used to introduce a broad range of genetic modifications by inducing DNA double-strand breaks that stimulate error-prone nonhomologous end joining or homology-directed repair at specific genomic locations. The versatility of ZFNs and TALENs arises from the ability to customize the DNA-binding domain to recognize virtually any sequence. These DNA-binding modules can be combined with numerous effector domains to affect genomic structure and function, including nucleases, transcriptional activators and repressors, recombinases, transposases, DNA and histone methyltransferases, and histone acetyltransferases. Thus, the ability to execute genetic alterations depends largely on the DNA-binding specificity and affinity of designed zinc finger and TALEN proteins (Gaj et al., Trends in Biotechnology, (2013) 31(7):397-405). The following U.S. granted patents, incorporated by reference, describe the use of ZFNs and TALENs in mammalian cells, U.S. Pat. Nos. 8,685,737 and 8,697,853.

In one aspect, the invention uses a ZFN or TALEN to insert nucleotide bases into the genome of a Whole Organism Control.

### CRISPR

CRISPR-Cas (clustered regularly interspaced short palindromic repeats-CRISPR associated) is an RNA-mediated adaptive immune system found in bacteria and archaea, which provides adaptive immunity against foreign nucleic acids (Wiedenheft et al., Nature (2012) 482:331-8; Jinek et al., Science (2012) 337:816-21). However, multiple research groups have demonstrated how the biological components of this system can be harnessed to introduce directed modification to the genome of mammalian cells. CRISPR-Cas systems are generally defined by a genomic locus called the CRISPR array, a series of 20-50 base-pair (bp) direct repeats separated by unique “spacers” of similar length and preceded by an AT-rich “leader” sequence (Wright et al., Cell (2016) 164:29-44).

Three types of CRISPR/Cas systems exist, type I, II and III. The Type II CRISPR-Cas systems require a single protein, Cas9, to catalyze DNA cleavage (Sapranauskas et al., Nucleic Acids Res. (2011) 39(21): 9275-9282). Cas9 serves as an RNA-guided DNA endonuclease. Cas9 generates blunt double-strand breaks (DSBs) at sites defined by a 20-nucleotide guide sequence contained within an associated CRISPR RNA (crRNA) transcript. Cas9 requires both the guide crRNA and a trans-activating crRNA (tracrRNA) that is partially complementary to the crRNA for site-specific DNA recognition and cleavage (Deltcheva et al., Nature (2011)4 71(7340):602-7; Jinek et al., Science (2012) 337:816-21).

Recent experiments showed that the crRNA:tracrRNA complex can be synthesized as a single transcript (single-guide RNA or sgRNA) encompassing the features required for both Cas9 binding and DNA target site recognition. Using sgRNA, Cas9 from Streptococcus pyogenes can be programmed to cleave double-stranded DNA at any site defined by the guide RNA sequence and including a GG protospacer-adjacent (PAM) motif (Sapranauskas et al., Nucleic Acids Res. (2011) 39(21): 9275-9282; Jinek et al., Science (2012) 337:816-21). Cas9 from other bacterial species utilize alternative PAM sequences, thereby increasing the CRISPR-targetable loci. The DSBs instigate either non-homologous end-joining (NHEJ), which is error-prone and conducive to frameshift mutations that knock out gene alleles, or homology-directed repair (HDR), which can be exploited with the use of an exogenously introduced double-strand or single-strand DNA repair template to knock in or correct a mutation in the genome. Therefore, in the presence of a homologous repair donor, the CRISPR/Cas9 system may be used to generate precise and defined modifications and insertions at a targeted locus through the HDR process. In the absence of a homologous repair donor, single DSBs generated by CRISPR/Cas9 are repaired through the error-prone NHEJ, which results in insertion or deletion (indel) mutations.

Other publications describing the CRISPR systems and Cas9, include the following Cong et al. Science (2013) 339:819-23; Jinek et al., eLife 2013; 2:e00471. (2013) 2:e00471; Lei et al. Cell (2013) 152: 1173-1183; Gilbert et al. Cell (2013) 154:442-51; Lei et al. eLife (2014) 3:e04766; Perez-Pinela et al. Nat Methods (2013) 10: 973-976; Maider et al. Nature Methods (2013) 10, 977-979 which are incorporated by reference. The following U.S. and international patents and patent applications describe the methods of use of CRISPR, 8,697,359; 8,771,945; 8,795,965; 8,865,406; 8,871,445; 8,889,356; 8,895,308; 8,906,616; 8,932,814; 8,945,839; 8,993,233; 8,999,641; 2014/0068797; and WO 2014/197568, all incorporated by reference in their entirety.

The CRISPR related protein, Cas9, can be from any number of species including, but not limited to, Streptococcus pyogenes, Listeria innocua, and Streptococcus thermophilus.

In one aspect, the invention uses a CRISPR-Cas system and one or more guide RNAs, repair templates and HDR to insert nucleotide bases into the genome of a Whole Organism Control.

In various embodiments, the genetic modification is carried out on a second nucleic acid that may be introduced into an organism, and encodes all or part of a gene selected from the group consisting of an antibiotic resistance gene, a drug resistance gene, a pathogenicity-determining gene, a genus- or species- or clade- or subtype-determining gene, an environmental response determining gene, a pathogen resistance determining gene, a growth-determining gene, an immune resistance or immune escape determining gene, a regulatory gene, a gene determining genetic or expression control, a genetic element encoding an interfering RNA (iRNA), a noncoding genetic segment or segment of unknown function, a biomarker, a virus gene, an oncogene, or a tumor suppressor gene.

### Antibiotic Resistance

In some embodiments the quality control composition of the present invention is designed to assess the functionality of a NAT used for detecting a target nucleic acid sequence that confers antibiotic resistance to a bacterium organism. In such instances the quality control composition of the present invention comprises a protected nucleic acid sequence (herein referred to as “second protected nucleic acid sequence”) that is genetically modified to comprise a gene, or more preferably an inactivated gene, or characteristic portion of a gene that is known or suspected of, alone or together with other genes or peptides (e.g. enzymes or activating or expression factors) to confer antibiotic resistance to the protected vehicle, said being capable of being detected by the NAT. The quality control composition of the present invention, in some aspects, may comprise more than one type of second nucleic acid sequence characteristic of the same or different antibiotic resistance associated gene.

In various embodiments, the protecting vehicle, such as an organism, in a quality control composition of the invention, is genetically modified to comprise one or more antibiotic resistance genes, in one embodiment preferably inactivated genes or nucleic acid sequences characteristic of said genes, including, but not limited to, aac2ia, aac2ib, aac2ic, aac2id, aac2i, aac3ia, aac3iia, aac3iib, aac3iii, aac3iv, aac3ix, aac3vi, aac3viii, aac3vii, aac3x, aac6i, aac6ia, aac6ib, aac6ic, aac6ie, aac6if, aac6ig, aac6iia, aac6iib, aad9, aad9ib, aadd, acra, acrb, adea, adeb, adec, amra, amrb, ant2ia, ant2ib, ant3ia, ant4iia, ant6ia, aph33ia, aph33ib, aph3ia, aph3ib, aph3ic, aph3iiia, aph3iva, aph3va, aph3vb, aph3via, aph3viia, aph4ib, aph6ia, aph6ib, aph6ic, aph6id, arna, baca, bcra, bcrc, bl1_acc, bl1_ampc, bl1_asba, bl1_ceps, bl1_cmy2, bl1_ec, bl1_fox, bl1_mox, bl1_och, bl1_pao, bl1_pse, bl1_sm, bl2a_1, bl2a_exo, bl2a_iii2, bl2a_iii, bl2a_kcc, bl2a_nps, bl2a_okp, bl2a_pc, bl2be_ctxm, bl2be_oxy1, bl2be_per, bl2be_shv2, bl2b_rob, bl2b_tem1, bl2b_tem2, bl2b_tem, bl2b_tle, bl2b_ula, bl2c_bro, bl2c_pse1, bl2c_pse3, bl2d_lcr1, bl2d_moxa, bl2d_oxa10, bl2d_oxa1, bl2d_oxa2, bl2d_oxa5, bl2d_oxa9, bl2d_r39, bl2e_cbla, bl2e_cepa, bl2e_cfxa, bl2e_fpm, bl2e_y56, bl2f_nmca, bl2f_sme1, bl2_ges, bl2_kpc, bl2_len, bl2_veb, bl3_ccra, bl3_cit, bl3_cpha, bl3_gim, bl3_imp, bl3_l, bl3_shw, bl3_sim, bl3_vim, ble, blt, bmr, cara, cata10, cata11, cata12, cata13, cata14, cata15, cata16, cata1, cata2, cata3, cata4, cata5, cata6, cata7, cata8, cata9, catb1, catb2, catb3, catb4, catb5, ceoa, ceob, cml_e1, cml_e2, cml_e3, cml_e4, cml_e5, cml_e6, cml_e7, cml_e8, dfra10, dfra12, dfra13, dfra14, dfra15, dfra16, dfra17, dfra19, dfra1, dfra20, dfra2l, dfra22, dfra23, dfra24, dfra25, dfra25, dfra25, dfra26, dfra5, dfra7, dfrb1, dfrb2, dfrb3, dfrb6, emea, emrd, emre, erea, ereb, erma, ermb, ermc, ermd, erme, ermf, ermg, ermh, ermn, ermo, ermq, ermr, erms, ermt, ermu, ermv, ermw, ermx, ermy, fosa, fosb, fosc, fosx, fusb, fush, ksga, lmra, lmrb, lnua, lnub, lsa, maca, macb, mdte, mdtf, mdtg, mdth, mdtk, mdtl, mdtm, mdtn, mdto, mdtp, meca, mecr1, mefa, mepa, mexa, mexb, mexc, mexd, mexe, mexf, mexh, mexi, mexw, mexx, mexy, mfpa, mpha, mphb, mphc, msra, norm, oleb, opcm, opra, oprd, oprj, oprm, oprn, otra, otrb, pbp1a, pbp1b, pbp2b, pbp2, pbp2x, pmra, qac, qaca, qacb, qnra, qnrb, qnrs, rosa, rosb, smea, smeb, smec, smed, smee, smef, srmb, sta, str, sul1, sul2, sul3, tcma, tcr3, tet30, tet31, tet32, tet33, tet34, tet36, tet37, tet38, tet39, tet40, teta, tetb, tetc, tetd, tete, tetg, teth, tetj, tetk, tetl, tetm, teto, tetpa, tetpb, tet, tetq, tets, tett, tetu, tetv, tetw, tetx, tety, tetz, tlrc, tmrb, toic, tsnr, vana, vanb, vanc, vand, vane, yang, vanha, vanhb, vanhd, vanra, vanrb, vanrc, vanrd, vanre, vanrg, vansa, vansb, vansc, vansd, vanse, vansg, vant, vante, vantg, vanug, vanwb, vanwg, vanxa, vanxb, vanxd, vanxyc, vanxye, vanxyg, vanya, vanyb, vanyd, vanyg, vanz, vata, vatb, vatc, vatd, vate, vgaa, vgab, vgba, vgbb, vph, ykkc, and ykkd.

### Inactivation Methods

Organisms used herein may be inactivated. Methods of inactivation include, but are not limited to, electromagnetic irradiation, infrared irradiation, visible light irradiation, laser irradiation, irradiation by a lamp, bulb, tube, filament, antenna, light-emitting diode, particle accelerator or other device, irradiation with radiation produced by radioisotopes, irradiation with radiation produced by electrical or electronic devices, natural irradiation, artificial irradiation, ultraviolet irradiation, alpha irradiation, beta irradiation, gamma irradiation, or X-ray irradiation, solvent treatment, detergent treatment, pasteurization, acidic pH inactivation, basic pH inactivation, covalent chemical modification of nucleic acids, covalent chemical modification of proteins, noncovalent chemical modification of nucleic acids, noncovalent chemical modification of proteins, chaotropic disruption, treatment with formalin, formaldehyde, paraformaldehyde, glutaraldehyde, ortho-phthalaldehyde, other aldehydes, beta propiolactone, hydrogen peroxide, other peroxides, chlorine and chlorine compounds, ammonia and ammonium compounds, amines, amine-reactive compounds, carboxylic compounds and carboxyl-reactive compounds, peracetic acid, phenolic compounds, and other chemically or physically reactive or disruptive compounds, physical disruption, freeze-thaw disruption, heat inactivation, pasteurization, other temperature-based and/or time based inactivation, and other means.

Previous publications describe methods for the inactivation of viruses, which may be pathogenic, for use in molecular assays wherein the viruses are inactivated by means of irreversible covalent modification of proteins, or by enzyme-mediated modification of proteins, either of which may result in changes that render such viruses non-pathogenic (see for example CA 2,426,172). The method of CA 2,426,172 is directed to the treatment of viruses by such means while rendering them “substantially intact”, allowing for minimal contact of the microorganism's intracellular components with the modifying agents so as to preserve enough of the nuclear contents, in particular the nucleic acid content, from degradation by the modifying reagents such that the nucleic acid is amenable to nucleic acid amplification techniques; CA 2,426,172 is hereby incorporated in its entirety by reference. In some embodiments, the method requires the use of chemicals (for example formalin) that may be poisonous and/or carcinogenic to humans at certain levels. In some aspects, the method of the present invention utilizes procedures for the inactivation of viruses resulting in a form that is “substantially intact”; one of many methods known to those skilled in the art (and as described elsewhere herein), optionally, is the method of CA 2,426,172.

Various studies describe the use of irradiation for the inactivation of pathogens. For example, Elliott et al., J. Clin. Microbiol. 1982, vol. 16 no. 4, 704-708, describe the use of ultraviolet or gamma irradiation to inactivate viruses. However, it is generally understood that such irradiative treatments cause inactivation through damage to nucleic acids. For example, Simonet et al. Appl. Environ. Microbiol. 2006, vol. 72 no. 12 7671-7677, showed that ultraviolet light caused the fragmentation of the genome of poliovirus and RNA Phages as the major mechanism of the induction of inactivation. Similarly, G. P. Van Der Schans & J. F. Bleichrodt (1974) Int. J. Rad. Biol Relat. Stud. Phys. Chem. Med., 26:2, 121-126 have shown that the majority of damage in gamma irradiation inactivation of bacteriophage PM2 DNA derives from damage to nucleic acids.

The method of irradiation may be chosen from one or more of the following: electromagnetic irradiation, infrared irradiation, visible light irradiation, laser irradiation, irradiation by a lamp, bulb, tube, filament, antenna, light-emitting diode, particle accelerator or other device, irradiation with radiation produced by radioisotopes, irradiation with radiation from electrical or electronic devices, natural irradiation, artificial irradiation, ultraviolet irradiation, alpha irradiation, beta irradiation, gamma irradiation, or X-ray irradiation. In some aspects, methods for inactivating pathogens for use as molecular controls using electromagnetic radiation, including, gamma irradiation, at a level that inactivates the biological activity of the control while leaving the sample DNA sufficiently intact for processing and analysis can be used.

### Whole Organisms Controls

In various embodiments of the invention, the method is practicable using a variety of organisms, including with a eukaryote, bacterium, virus, fungus, archaeon, protist, or parasite whether pathogenic or not, whether naturally occurring or synthetic (for example recombinant). In some cases, the organisms used as Control Materials are pathogenic organisms. In some cases, the organisms used as Control Materials are non-pathogenic. In some cases, the organisms used as Control Materials contain nucleic acid, either native or not native to the organism, that differentiate them from other organisms of the same general type or species (for example, Staphylococcus Aureus organisms, admixed with Escherichia coli that naturally or by way of a genetic modification contain genetic sequences characteristic of the mecA gene, or Staphylococcus Aureus organisms, genetically modified to include a part of the mecA gene).

In some cases, the organisms used as Controls contain genetic targets that differentiate them from other organisms of the same general type or species (for example, human cells bearing a cancer-associated genetic sequence). A partial list of organisms that may be treated according to the method of the invention, includes for example, and not by way of limitation: Acinetobacter baumannii, Adenovirus F40/41, Astrovirus, Bacillus Anthracis, Babesia (Nuttalia), Bordetella pertussis, Borelia, Candida tropicalis, Candida albicans, Candida parapsilosis, Candida glabrata, Chikungunya, Candida krusei, Clostridium difficile, Campylobacter (jejuni, coli and upsaliensis), Candida albicans, Clostridium difficile (toxin A/B), Coronavirus HKU1, Coronavirus NL63, Coronavirus 229E, Coronavirus OC43, Coxiella burnetii, Chlamydophila pneumoniae, Chlamydia trachomatis, Cryptococcus albidus, Cryptosporidium, Cyclospora cayetanensis, Cytomegalovirus, Dengue virus, Ebola virus, Rubella virus, Entercoccus, Entamoeba histolytica, Enterovirus, Escherichia coli, Escherichia coli, Escherichia coli K1, Enteroaggregative E. coli (EAEC), Enteropathogenic E. coli (EPEC), Enterotoxigenic E. coli (ETEC) lt/st, Shiga-like toxin-producing E. coli (STEC) stx1/stx2, E. coli 0157, Shigella/Enteroinvasive E. coli (EIEC), vancomycin resistant E. coli (VREC), Enterobacteriaceae, Enterobacter cloacae complex, Epstein Barr Virus, Giardia lamblia, Helicobacter pylori, Listeria monocytogenes, Klebsiella pneumoniae, Plasmodium (Malaria), Pseudomonas aeroginosa, Francisella tularensis, Gram-Positive, Gram-Negative Bacteria, Haemophilus influenzae, Herpes Simplex Virus, Herpes simplex virus 1 (HSV-1), Herpes simplex virus 2 (HSV-2), Hepatitis virus, Human Immunodeficiency Viruses, Human Metapneumovirus, Human Rhinovirus/Enterovirus, Human herpesvirus 6 (HHV-6), Human parechovirus, Human Metapneumovirus, Human papillomavirus, Haemophilus influenzae, Influenza and Respiratory Viruses, Influenza A, Influenza A/H1, Influenza A/H3, Influenza A/H1-2009, Influenza B, Parainfluenza Virus 1, Parainfluenza Virus 2, Parainfluenza Virus 3, Parainfluenza Virus 4, Respiratory Syncytial Virus, Japanese Encephalitis Virus, Klebsiella oxytoca, Klebsiella pneumoniae, Leishmania species, Measles, Mumps, Mycobacterium tuberculosis, Mycobacterium species, Mycoplasma genitalium, Mycoplasma pneumoniae, Multiplex Panel, Neisseria gonorrhoeae, Neisseria meningitidis, Norovirus GI/GII, Picchia pastoris, Plesiomonas shigelloides, Pseudomonas aeruginosa, Proteus, Rotavirus A, Saccharomyces cerevisiae, Sapovirus (I, II, IV & V), Salmonella, Serratia marcescens, Staphylococcus, Staphylococcus aureus, Streptococcus, Streptococcus agalactiae, Streptococcus pneumoniae, Streptococcus pyogenes, Staphylococcus, Streptococci, Trichomonas vaginalis, Varicella zoster virus (VZV), Vibrio (parahaemolyticus, vulnificus and cholerae), Vibrio cholerae, Zika virus, West Nile Virus, Yellow Fever Virus, Yersinia enterocolitica, Yersinia pestis; Human cells, human cells with genetic traits of interest, human cells infected with pathogenic or nonpathgenic organisms; plant, animal, fungus, or monera cells; plant, animal, fungus, or monera cells infected with pathogenic or non-pathogenic organisms; plant, animal, fungus, or monera cells with genetic traits of interest; plant, animal, fungus, or monera cells infected with pathogenic or non-pathogenic organisms; organisms comprising oncogenes; organisms comprising heritable genetic traits including traits indicative of disease, organisms containing exosomal nucleic acids.

Exemplary bacteria that can be identified using the present methods, or used as a whole Organism Control, include, but are not limited to, Acetobacter aurantius, Acinetobacter species: Acinetobacter baumannii, Acinetobacter calcoaceticus, Acinetobacter johnsonii, Acinetobacter junii, Acinetobacter iwoffii, Acinetobacter radioresistens, Acinetobacter septicus, Acinetobacter schindleri, Acinetobacter ursingii; Actinomyces species: Actinomyces bovis, Actinomyces bowdenii, Actinomyces canis, Actinomyces cardiffensis, Actinomyces catuli, Actinomyces coleocanis, Actinomyces dentalis, Actinomyces denticolens, Actinomyces europaeus, Actinomyces funkei, Actinomyces georgiae, Actinomyces gerencseriae, Actinomyces graevenitzii, Actinomyces hongkongensis, Actinomyces hordeovulneris, Actinomyces howellii, Actinomyces humiferus, Actinomyces hyovaginalis, Actinomyces israelii, Actinomyces marimammalium, Actinomyces meyeri, Actinomyces naeslundii, Actinomyces nasicola, Actinomyces neuii, Actinomyces odontolyticus, Actinomyces oricola, Actinomyces radicidentis, Actinomyces radingae, Actinomyces slackii, Actinomyces streptomycini, Actinomyces suimastitidis, Actinomyces suis, Actinomyces turicensis, Actinomyces urogenitalis, Actinomyces vaccimaxillae, Actinomyces viscosus; Actinobacillus species: Actinobacillus actinomycetemcomitans, Actinobacillus arthritidis, Actinobacillus capsulatus, Actinobacillus delphinicola, Actinobacillus equuli, Actinobacillus hominis, Actinobacillus indolicus, Actinobacillus lignieresii, Actinobacillus minor, Actinobacillus muris, Actinobacillus pleuropneumoniae, Actinobacillus porcinus, Actinobacillus rossii, Actinobacillus scotiae, Actinobacillus seminis, Actinobacillus succinogenes, Actinobacillus suis, Actinobacillus ureae; Aeromonas species: Aeromonas allosaccharophila, Aeromonas bestiarum, Aeromonas bivalvium, Aeromonas encheleia, Aeromonas enteropelogenes, Aeromonas euchrenophila, Aeromonas hydrophila, Aeromonas ichthiosmia, Aeromonas jandaei, Aeromonas media, Aeromonas molluscorum, Aeromonas popoffii, Aeromonas punctata, Aeromonas salmonicida, Aeromonas schubertii, Aeromonas sharmana, Aeromonas simiae, Aeromonas sobria, Aeromonas veronii; Afipia fells, Agrobacterium species: Agrobacterium radiobacter, Agrobacterium rhizogenes, Agrobacterium rubi, Agrobacterium tumefaciens; Agromonas species, Alcaligenes species: Alcaligenes aquatilis, Alcaligenes eutrophus, Alcaligenes faecalis, Alcaligenes latus, Alcaligenes xylosoxidans; Alishewanella species, Alterococcus species, Anaplasma phagocytophilum, Anaplasma marginale, Aquamonas species, Arcanobacterium haemolyticum, Aranicola species, Arsenophonus species, Azotivirga species, Azotobacter vinelandii, Azotobacter chroococcum, Bacillary dysentery (Shigellosis), Bacillus species: Bacillus abortus (Brucella melitensis biovar abortus), Bacillus anthracis (Anthrax), Bacillus brevis, Bacillus cereus, Bacillus coagulans, Bacillus fusiformis, Bacillus globigii, Bacillus licheniformis, Bacillus megaterium, Bacillus mycoides, Bacillus natto, Bacillus stearothermophilus, Bacillus subtilis, Bacillus sphaericus, Bacillus thuringiensis; Bacteroides species: Bacteroides forsythus (Tannerella forsythensis), Bacteroides acidifaciens, Bacteroides distasonis (reclassified as Parabacteroides distasonis), Bacteroides gingivalis, Bacteroides gracilis, Bacteroides fragilis, Bacteroides oris, Bacteroides ovatus, Bacteroides putredinis, Bacteroides pyogenes, Bacteroides stercoris, Bacteroides suis, Bacteroides tectus, Bacteroides thetaiotaomicron, Bacteroides vulgatus, Bartonella species: Bartonella alsatica, Bartonella bacilliformis, Bartonella birtlesii, Bartonella bovis, Bartonella capreoli, Bartonella clarridgeiae, Bartonella doshiae, Bartonella elizabethae, Bartonella grahamii, Bartonella henselae (cat scratch fever), Bartonella koehlerae, Bartonella muris, Bartonella peromysci, Bartonella quintana, Bartonella rochalimae, Bartonella schoenbuchii, Bartonella talpae, Bartonella taylorii, Bartonella tribocorum, Bartonella vinsonii spp. Arupensis, Bartonella vinsonii spp. Berkhoffii, Bartonella vinsonii spp. Vinsonii, Bartonella washoensis; BCG (Bacille Calmette-Guerin), Bergeyella zoohelcum (Weeksella zoohelcum), Bifidobacterium bifidum, Blastobacter species, Blochmannia species, Bordetella species: Bordetella ansorpii, Bordetella avium, Bordetella bronchiseptica, Bordetella hinzii, Bordetella holmesii, Bordetella parapertussis, Bordetella pertussis (Whooping cough), Bordetella petrii, Bordetella trematum; Borrelia species: Borrelia burgdorferi, Borrelia afzelii, Borrelia anserina, Borrelia garinii, Borrelia valaisiana, Borrelia hermsii, Borrelia Parkeri, Borrelia recurrentis; Bosea species, Bradyrhizobium species, Brenneria species, Brucella species: Brucella abortus, Brucella canis, Brucella melitensis, Brucella neotomae, Brucella ovis, Brucella suis, Brucella pinnipediae; Buchnera species, Budvicia species, Burkholderia species: Burkholderia cepacia (Pseudomonas cepacia), Burkholderia mallei (Pseudomonas mallei/Actinobacillus mallei), Burkholderia pseudomallei (Pseudomonas pseudomallei); Buttiauxella species, Calymmatobacterium granulomatis, Campylobacter species: Campylobacter coli, Campylobacter concisus, Campylobacter curvus, Campylobacter fetus, Campylobacter gracilis, Campylobacter helveticus, Campylobacter hominis, Campylobacter hyointestinalis, Campylobacter insulaenigrae, Campylobacter jejuni, Campylobacter lanienae, Campylobacter lari, Campylobacter mucosalis, Campylobacter rectus, Campylobacter showae, Campylobacter sputorum, Campylobacter upsaliensis; Capnocytophaga canimorsus (Dysgonic fermenter type 2), Corynebacterium species, Cardiobacterium hominis, Cedecea species, Chlamydia species: Chlamydia trachomatis (Lymphogranuloma venereum), Chlamydia muridarum, Chlamydia suis; Chlamydophila species: Chlamydophila pneumoniae, Chlamydophila psittaci (Psittacosis), Chlamydophila pecorum, Chlamydophila abortus, Chlamydophila felis, Chlamydophila caviae; Citrobacter species: Citrobacter amalonaticus, Citrobacter braakii, Citrobacter farmeri, Citrobacter freundii, Citrobacter gillenii, Citrobacter intermedius, Citrobacter koseri aka Citrobacter diversus, Citrobacter murliniae, Citrobacter rodentium, Citrobacter sedlakii, Citrobacter werkmanii, Citrobacter youngae; Clostridium species: Clostridium botulinum, Clostridium difficile, Clostridium novyi, Clostridium septicum, Clostridium tetani (Tetanus), Clostridium welchii (Clostridium perfringens); Corynebacterium species: Corynebacterium diphtheriae (Diphtheria), Corynebacterium amycolatum, Corynebacterium aquaticum, Corynebacterium bovis, Corynebacterium equi, Corynebacterium flavescens, Corynebacterium glutamicum, Corynebacterium haemolyticum, Corynebacterium jeikeiun (corynebacteria of group JK), Corynebacterium minutissimum (Erythrasma), Corynebacterium parvum (also called Propionibacterium acnes), Corynebacterium pseudodiptheriticum (also called Corynebacterium hofmannii), Corynebacterium pseudotuberculosis (also called Corynebacterium ovis), Corynebacterium pyogenes, Corynebacterium urealyticum (corynebacteria of group D2), Corynebacterium renale, Corynebacterium striatum, Corynebacterium tenuis (Trichomycosis palmellina, Trichomycosis axillaris), Corynebacterium ulcerans, Corynebacterium xerosis; Coxiella burnetii (Q fever), Cronobacter species: Cronobacter sakazakii, Cronobacter malonaticus, Cronobacter turicensis, Cronobacter muytjensii, Cronobacter dublinensis; Delftia acidovorans (Comamonas acidovorans), Dickeya species, Edwardsiella species, Eikenella corrodens, Enterobacter species: Enterobacter aerogenes, Enterobacter cloacae, Enterobacter sakazakii; Enterococcus species: Enterococcus avium, Enterococcus durans, Enterococcus faecalis (Streptococcus faecalis/Streptococcus Group D), Enterococcus faecium, Enterococcus solitarius, Enterococcus galllinarum, Enterococcus maloratus; Ehrlichia chaffeensis, Erysipelothrix rhusiopathiae, Erwinia species, Escherichia species: Escherichia adecarboxylata, Escherichia albertii, Escherichia blattae, Escherichia coli, Escherichia fergusonii, Escherichia hermannii, Escherichia vulneris; Ewingella species, Flavobacterium species: Flavobacterium aquatile, Flavobacterium branchiophilum, Flavobacterium columnare, Flavobacterium flevense, Flavobacterium gondwanense, Flavobacterium hydatis, Flavobacterium johnsoniae, Flavobacterium pectinovorum, Flavobacterium psychrophilum, Flavobacterium saccharophilum, Flavobacterium salegens, Flavobacterium scophthalmum, Flavobacterium succinans; Francisella tularensis (Tularaemia), Francisella novicida, Francisella philomiragia, Fusobacterium species: Fusobacterium necrophorum (Lemierre syndrome/Sphaerophorus necrophorus), Fusobacterium nucleatum, Fusobacterium polymorphum, Fusobacterium novum, Fusobacterium mortiferum, Fusobacterium varium; Gardnerella vaginalis, Gemella haemolysans, Gemella morbillorum (Streptococcus morbillorum), Grimontella species, Haemophilus species: Haemophilus aegyptius (Koch-Weeks bacillus), Haemophilus aphrophilus, Haemophilus avium, Haemophilus ducreyi (Chancroid), Haemophilus felis, Haemophilus haemolyticus, Haemophilus influenzae (Pfeiffer bacillus), Haemophilus paracuniculus, Haemophilus parahaemolyticus, Haemophilus parainfluenzae, Haemophilus paraphrophilus (Aggregatibacter aphrophilus), Haemophilus pertussis, Haemophilus pittmaniae, Haemophilus somnus, Haemophilus vaginalis; Hafnia species, Hafnia alvei, Helicobacter species: Helicobacter acinonychis, Helicobacter anseris, Helicobacter aurati, Helicobacter bilis, Helicobacter bizzozeronii, Helicobacter brantae, Helicobacter Canadensis, Helicobacter canis, Helicobacter cholecystus, Helicobacter cinaedi, Helicobacter cynogastricus, Helicobacter felis, Helicobacter fennelliae, Helicobacter ganmani, Helicobacter heilmannii (Gastrospirillum hominis), Helicobacter hepaticus, Helicobacter mesocricetorum, Helicobacter marmotae, Helicobacter muridarum, Helicobacter mustelae, Helicobacter pametensis, Helicobacter pullorum, Helicobacter pylori (stomach ulcer), Helicobacter rappini, Helicobacter rodentium, Helicobacter salomonis, Helicobacter trogontum, Helicobacter typhlonius, Helicobacter winghamensis; Human granulocytic ehrlichiosis (Anaplasma phagocytophilum/Ehrlichia phagocytophila), Human monocytotropic ehrlichiosis (Monocytic ehrlichiosis/Ehrlichia chaffeensis), Klebsiella species: Klebsiella granulomatis (Calymmatobacterium granulomatis), Klebsiella mobilis, Klebsiella ornithinolytica, Klebsiella oxytoca, Klebsiella ozaenae, Klebsiella planticola, Klebsiella pneumoniae, Klebsiella rhinoscleromatis, Klebsiella singaporensis, Klebsiella terrigena, Klebsiella trevisanii, Klebsiella variicola; Kingella kingae, Kluyvera species, Lactobacillus species: Lactobacillus acetotolerans, Lactobacillus acidifarinae, Lactobacillus acidipiscis, Lactobacillus acidophilus (Doderlein bacillus), Lactobacillus agilis, Lactobacillus algidus, Lactobacillus alimentarius, Lactobacillus amylolyticus, Lactobacillus amylophilus, Lactobacillus amylotrophicus, Lactobacillus amylovorus, Lactobacillus animalis, Lactobacillus antri, Lactobacillus apodemi, Lactobacillus aviarius, Lactobacillus bifermentans, Lactobacillus brevis, Lactobacillus buchneri, Lactobacillus camelliae, Lactobacillus casei, Lactobacillus catenaformis, Lactobacillus ceti, Lactobacillus coleohominis, Lactobacillus collinoides, Lactobacillus composti, Lactobacillus concavus, Lactobacillus coryniformis, Lactobacillus crispatus, Lactobacillus crustorum, Lactobacillus curvatus, Lactobacillus delbrueckii, Lactobacillus delbrueckii subsp. Bulgaricus, Lactobacillus delbrueckii subsp. Lactis, Lactobacillus diolivorans, Lactobacillus equi, Lactobacillus equigenerosi, Lactobacillus farraginis, Lactobacillus farciminis, Lactobacillus fermentum, Lactobacillus formicalis, Lactobacillus fructivorans, Lactobacillus frumenti, Lactobacillus fuchuensis, Lactobacillus gallinarum, Lactobacillus gasseri, Lactobacillus gastricus, Lactobacillus ghanensis, Lactobacillus graminis, Lactobacillus hammesii, Lactobacillus hamsteri, Lactobacillus harbinensis, Lactobacillus hayakitensis, Lactobacillus helveticus, Lactobacillus hilgardii, Lactobacillus homohiochii, Lactobacillus iners, Lactobacillus ingluviei, Lactobacillus intestinalis, Lactobacillus jensenii, Lactobacillus johnsonii, Lactobacillus kalixensis, Lactobacillus kefuranofaciens, Lactobacillus kefiri, Lactobacillus kimchii, Lactobacillus kitasatonis, Lactobacillus kunkeei, Lactobacillus leichmannii, Lactobacillus lindneri, Lactobacillus malefermentans, Lactobacillus mall, Lactobacillus manihotivorans, Lactobacillus mindensis, Lactobacillus mucosae, Lactobacillus murinus, Lactobacillus nagelii, Lactobacillus namurensis, Lactobacillus nantensis, Lactobacillus oligofermentans, Lactobacillus oris, Lactobacillus panis, Lactobacillus pantheris, Lactobacillus parabrevis, Lactobacillus parabuchneri, Lactobacillus paracollinoides, Lactobacillus parafarraginis, Lactobacillus parakefiri, Lactobacillus paralimentarius, Lactobacillus paraplantarum, Lactobacillus pentosus, Lactobacillus perolens, Lactobacillus plantarum, Lactobacillus pontis, Lactobacillus psittaci, Lactobacillus rennini, Lactobacillus reuteri, Lactobacillus rhamnosus, Lactobacillus rimae, Lactobacillus rogosae, Lactobacillus rossiae, Lactobacillus ruminis, Lactobacillus saerimneri, Lactobacillus sakei, Lactobacillus salivarius, Lactobacillus sanfranciscensis, Lactobacillus satsumensis, Lactobacillus secaliphilus, Lactobacillus sharpeae, Lactobacillus siliginis, Lactobacillus spicheri, Lactobacillus suebicus, Lactobacillus thailandensis, Lactobacillus ultunensis, Lactobacillus vaccinostercus, Lactobacillus vaginalis, Lactobacillus versmoldensis, Lactobacillus vini, Lactobacillus vitulinus, Lactobacillus zeae, Lactobacillus zymae; Leclercia species, Legionella species: Legionella adelaidensis, Legionella anisa, Legionella beliardensis, Legionella birminghamensis, Legionella bozemanii, Legionella brunensis, Legionella busanensis, Legionella cherrii, Legionella cincinnatiensis, Legionella donaldsonii, Legionella drancourtii, Legionella drozanskii, Legionella erythra, Legionella fairfieldensis, Legionella fallonii, Legionella feeleii, Legionella geestiana, Legionella genomospecies, Legionella gratiana, Legionella gresilensis, Legionella hackeliae, Legionella impletisoli, Legionella israelensis, Legionella jamestowniensis, ‘Candidatus Legionella jeonii’, Legionella jordanis, Legionella lansingensis, Legionella londiniensis, Legionella longbeachae, Legionella lytica, Legionella maceachernii, Legionella micdadei, Legionella moravica, Legionella nautarum, Legionella oakridgensis, Legionella parisiensis, Legionella pneumophila, Legionella quateirensis, Legionella quinlivanii, Legionella rowbothamii, Legionella rubrilucens, Legionella sainthelensi, Legionella santicrucis, Legionella shakespearei, Legionella spiritensis, Legionella steigerwaltii, Legionella taurinensis, Legionella tucsonensis, Legionella wadsworthii, Legionella waltersii, Legionella worsleiensis, Legionella yabuuchiae; Leminorella species, Leptospira species: Leptospira interrogans, Leptospira kirschneri, Leptospira noguchii, Leptospira alexanderi, Leptospira weilii, Leptospira genomospecies 1, Leptospira borgpetersenii, Leptospira santarosai, Leptospira inadai, Leptospira fainei, Leptospira broomii, Leptospira licerasiae, Leptospira biflexa, Leptospira meyeri, Leptospira wolbachii, Leptospira genomospecies 3, Leptospira genomospecies 4, Leptospira genomospecies 5; Lepromatous leprosy (Danielssen-Boeck disease), Leptospira canicola, Leptospira hebdomadis, Leptospirosis (Weil disease/Leptospira icterohaemorrhagiae/Leptospira interrogans serovar icterohaemorrhagiae), Leptotrichia, Leuconostoc species: Leuconostoc carnosum, Leuconostoc citreum, Leuconostoc durionis, Leuconostoc fallax, Leuconostoc ficulneum, Leuconostoc fructosum, Leuconostoc garlicum, Leuconostoc gasicomitatum, Leuconostoc gelidum, Leuconostoc inhae, Leuconostoc kimchii, Leuconostoc lactis, Leuconostoc mesenteroides, Leuconostoc pseudoficulneum, Leuconostoc pseudomesenteroides; Listeria species: Listeria grayi, Listeria innocua, Listeria ivanovii, Listeria monocytogenes (Listeriosis), Listeria seeligeri, Listeria welshimeri; Methanobacterium extroquens, Microbacterium multiforme, Micrococcus species: Micrococcus antarcticus, Micrococcus flavus, Micrococcus luteus, Micrococcus lylae, Micrococcus mucilaginosis, Micrococcus roseus, Micrococcus sedentarius; Mobiluncus, Moellerella species, Morganella species, Moraxella species: Moraxella atlantae, Moraxella boevrei, Moraxella bovis, Moraxella canis, Moraxella caprae, Moraxella catarrhalis (Branhamella catarrhalis), Moraxella caviae, Moraxella cuniculi, Moraxella equi, Moraxella lacunata, Moraxella lincolnii, Moraxella nonliquefaciens, Moraxella oblonga, Moraxella osloensis, Moraxella saccharolytica; Morganella morganii, Mycobacterium species: Mycobacterium abscessus, Mycobacterium africanum, Mycobacterium agri, Mycobacterium aichiense, Mycobacterium alvei, Mycobacterium arupense, Mycobacterium asiaticum, Mycobacterium aubagnense, Mycobacterium aurum, Mycobacterium austroafricanum, Mycobacterium avium (Battey disease/Lady Windermere syndrome), Mycobacterium avium paratuberculosis (implicated in Crohn's disease in humans and Johne's disease in sheep), Mycobacterium avium silvaticum, Mycobacterium avium “hominissuis”, Mycobacterium colombiense, Mycobacterium boenickei, Mycobacterium bohemicum, Mycobacterium bolletii, Mycobacterium botniense, Mycobacterium bovis (Bovine tuberculosis), Mycobacterium branderi, Mycobacterium brisbanense, Mycobacterium brumae, Mycobacterium canariasense, Mycobacterium caprae, Mycobacterium celatum, Mycobacterium chelonae, Mycobacterium chimaera, Mycobacterium chitae, Mycobacterium chlorophenolicum, Mycobacterium chubuense, Mycobacterium conceptionense, Mycobacterium confluentis, Mycobacterium conspicuum, Mycobacterium cookii, Mycobacterium cosmeticum, Mycobacterium diernhoferi, Mycobacterium doricum, Mycobacterium duvalii, Mycobacterium elephantis, Mycobacterium fallax, Mycobacterium farcinogenes, Mycobacterium flavescens, Mycobacterium florentinum, Mycobacterium fluoroanthenivorans, Mycobacterium fortuitum, Mycobacterium fortuitum subsp. Acetamidolyticum, Mycobacterium frederiksbergense, Mycobacterium gadium, Mycobacterium gastri, Mycobacterium genavense, Mycobacterium gilvum, Mycobacterium goodii, Mycobacterium gordonae (Mycobacterium aquae), Mycobacterium haemophilum, Mycobacterium hassiacum, Mycobacterium heckeshornense, Mycobacterium heidelbergense, Mycobacterium hiberniae, Mycobacterium hodleri, Mycobacterium holsaticum, Mycobacterium houstonense, Mycobacterium immunogenum, Mycobacterium interjectum, Mycobacterium intermedium, Mycobacterium intracellulare, Mycobacterium kansasii, Mycobacterium komossense, Mycobacterium kubicae, Mycobacterium kumamotonense, Mycobacterium lacus, Mycobacterium lentiflavum, Mycobacterium leprae (causes leprosy or Hansen disease/Hanseniasis), Mycobacterium lepraemurium, Mycobacterium madagascariense, Mycobacterium mageritense, Mycobacterium malmoense, Mycobacterium marinum (Fish tank granuloma), Mycobacterium massiliense, Mycobacterium microti, Mycobacterium monacense, Mycobacterium montefiorense, Mycobacterium moriokaense, Mycobacterium mucogenicum, Mycobacterium murale, Mycobacterium nebraskense, Mycobacterium neoaurum, Mycobacterium neworleansense, Mycobacterium nonchromogenicum, Mycobacterium novocastrense, Mycobacterium obuense, Mycobacterium palustre, Mycobacterium parafortuitum, Mycobacterium parascrofulaceum, Mycobacterium parmense, Mycobacterium peregrinum, Mycobacterium phlei, Mycobacterium phocaicum, Mycobacterium pinnipedii, Mycobacterium porcinum, Mycobacterium poriferae, Mycobacterium pseudoshottsii, Mycobacterium pulveris, Mycobacterium psychrotolerans, Mycobacterium pyrenivorans, Mycobacterium rhodesiae, Mycobacterium saskatchewanense, Mycobacterium scrofulaceum, Mycobacterium senegalense, Mycobacterium seoulense, Mycobacterium septicum, Mycobacterium shimoidei, Mycobacterium shottsii, Mycobacterium simiae, Mycobacterium smegmatis, Mycobacterium sphagni, Mycobacterium szulgai, Mycobacterium terrae, Mycobacterium thermoresistibile, Mycobacterium tokaiense, Mycobacterium triplex, Mycobacterium triviale, Mycobacterium tuberculosis (major cause of human tuberculosis), Mycobacterium bovis, Mycobacterium africanum, Mycobacterium canetti, Mycobacterium caprae, Mycobacterium pinnipedii’, Mycobacterium tusciae, Mycobacterium ulcerans (causes Bairnsdale ulcer/Buruli ulcer), Mycobacterium vaccae, Mycobacterium vanbaalenii, Mycobacterium wolinskyi, Mycobacterium xenopi; Mycoplasma species: Mycoplasma fermentans, Mycoplasma genitalium, Mycoplasma hominis, Mycoplasma penetrans, Mycoplasma phocacerebrale, Mycoplasma pneumoniae, Nanukayami (Seven-day fever/Gikiyami), Neisseria species: Neisseria gonorrhoea (Gonococcus/Gonorrhea), Neisseria meningiditis (Meningococcus), Neisseria sicca, Neisseria cinerea, Neisseria elongata, Neisseria flavescens, Neisseria lactamica, Neisseria mucosa, Neisseria polysaccharea, Neisseria subflava; Nitrobacter species, Nocardia species: Nocardia asteroides, Nocardia brasiliensis, Nocardia caviae; Noma (cancrum oris/gangrenous stomatitis), Obesumbacterium, Oligotropha species, Orientia tsutsugamushi (Scrub typhus), Oxalobacter formigenes, Pantoea species: Pantoea agglomerans, Pantoea ananatis, Pantoea citrea, Pantoea dispersa, Pantoea punctata, Pantoea stewartii, Pantoea terrea; Pasteurella species: Pasteurella aerogenes, Pasteurella anatis, Pasteurella avium, Pasteurella bettyae, Pasteurella caballi, Pasteurella canis, Pasteurella dagmatis, Pasteurella gallicida, Pasteurella gallinarum, Pasteurella granulomatis, Pasteurella langaaensis, Pasteurella lymphangitidis, Pasteurella mairii, Pasteurella multocida, Pasteurella pneumotropica, Pasteurella skyensis, Pasteurella stomatis, Pasteurella testudinis, Pasteurella trehalosi, Pasteurella tularensis, Pasteurella ureae, Pasteurella volantium; Pediococcus species: Pediococcus acidilactici, Pediococcus cellicola, Pediococcus claussenii, Pediococcus damnosus, Pediococcus dextrinicus, Pediococcus ethanolidurans, Pediococcus inopinatus, Pediococcus parvulus, Pediococcus pentosaceus, Pediococcus stilesii; Peptostreptococcus species: Peptostreptococcus anaerobius, Peptostreptococcus asaccharolyticus, Peptostreptococcus harei, Peptostreptococcus hydrogenalis, Peptostreptococcus indoliticus, Peptostreptococcus ivorii, Peptostreptococcus lacrimalis, Peptostreptococcus lactolyticus, Peptostreptococcus magnus, Peptostreptococcus micros, Peptostreptococcus octavius, Peptostreptococcus prevotii, Peptostreptococcus tetradius, Peptostreptococcus vaginalis; Photorhabdus species, Photorhizobium species, Plesiomonas shigelloides, Porphyromonas gingivalis, Pragia species, Prevotella, Propionibacterium species: Propionibacterium acnes, Propionibacterium propionicus; Proteus species: Proteus mirabilis, Proteus morganii, Proteus penneri, Proteus rettgeri, Proteus vulgaris; Providencia species: Providencia friedericiana, Providencia stuartii; Pseudomonas species: Pseudomonas aeruginosa, Pseudomonas alcaligenes, Pseudomonas anguilliseptica, Pseudomonas argentinensis, Pseudomonas borbori, Pseudomonas citronellolis, Pseudomonas flavescens, Pseudomonas mendocina, Pseudomonas nitroreducens, Pseudomonas oleovorans, Pseudomonas pseudoalcaligenes, Pseudomonas resinovorans, Pseudomonas straminea, Pseudomonas aurantiaca, Pseudomonas aureofaciens, Pseudomonas chlororaphis, Pseudomonas fragi, Pseudomonas lundensis, Pseudomonas taetrolens, Pseudomonas Antarctica, Pseudomonas azotoformans, Pseudomonas brassicacearum, Pseudomonas brenneri, Pseudomonas cedrina, Pseudomonas corrugate, Pseudomonas fluorescens, Pseudomonas gessardii, Pseudomonas libanensis, Pseudomonas mandelii, Pseudomonas marginalis, Pseudomonas mediterranea, Pseudomonas meridiana, Pseudomonas migulae, Pseudomonas mucidolens, Pseudomonas orientalis, Pseudomonas panacis, Pseudomonas proteolytica, Pseudomonas rhodesiae, Pseudomonas synxantha, Pseudomonas thivervalensis, Pseudomonas tolaasii, Pseudomonas veronii, Pseudomonas denitrificans, Pseudomonas pertucinogena, Pseudomonas cremoricolorata, Pseudomonas fulva, Pseudomonas monteilii, Pseudomonas mosselii, Pseudomonas oryzihabitans, Pseudomonas parafulva, Pseudomonas plecoglossicida, Pseudomonas putida, Pseudomonas balearica, Pseudomonas luteola, Pseudomonas stutzeri, Pseudomonas amygdale, Pseudomonas avellanae, Pseudomonas caricapapayae, Pseudomonas cichorii, Pseudomonas coronafaciens, Pseudomonas ficuserectae, Pseudomonas meliae, Pseudomonas savastanoi, Pseudomonas syringae, Pseudomonas viridiflava, Pseudomonas abietaniphila, Pseudomonas acidophila, Pseudomonas agarici, Pseudomonas alcaliphila, Pseudomonas alkanolytica, Pseudomonas amyloderamosa, Pseudomonas asplenii, Pseudomonas azotifigens, Pseudomonas cannabina, Pseudomonas coenobios, Pseudomonas congelans, Pseudomonas costantinii, Pseudomonas cruciviae, Pseudomonas delhiensis, Pseudomonas excibis, Pseudomonas extremorientalis, Pseudomonas frederiksbergensis, Pseudomonas fuscovaginae, Pseudomonas gelidicola, Pseudomonas grimontii, Pseudomonas indica, Pseudomonas jessenii, Pseudomonas jinjuensis, Pseudomonas kilonensis, Pseudomonas knackmussii, Pseudomonas koreensis, Pseudomonas lini, Pseudomonas lutea, Pseudomonas moraviensis, Pseudomonas otitidis, Pseudomonas pachastrellae, Pseudomonas palleroniana, Pseudomonas papaveris, Pseudomonas peli, Pseudomonas perolens, Pseudomonas poae, Pseudomonas pohangensis, Pseudomonas psychrophila, Pseudomonas psychrotolerans, Pseudomonas rathonis, Pseudomonas reptilivora, Pseudomonas resiniphila, Pseudomonas rhizosphaerae, Pseudomonas rubescens, Pseudomonas salomonii, Pseudomonas segitis, Pseudomonas septica, Pseudomonas simiae, Pseudomonas suis, Pseudomonas thermotolerans, Pseudomonas tremae, Pseudomonas trivialis, Pseudomonas turbinellae, Pseudomonas tuticorinensis, Pseudomonas umsongensis, Pseudomonas vancouverensis, Pseudomonas vranovensis, Pseudomonas xanthomarina; Rahnella species, Ralstonia species: Ralstonia basilensis, Ralstonia campinensis, Ralstonia eutropha, Ralstonia gilardii, Ralstonia insidiosa, Ralstonia mannitolilytica, Ralstonia metallidurans, Ralstonia paucula, Ralstonia pickettii, Ralstonia respiraculi, Ralstonia solanacearum, Ralstonia syzygii, Ralstonia taiwanensis; Raoultella species, Rhodoblastus species, Rhodopseudomonas species, Rhinoscleroma, Rhizobium radiobacter, Rhodococcus equi, Rickettsia species: Rickettsia africae, Rickettsia akari, Rickettsia australis, Rickettsia conorii, Rickettsia felis, Rickettsia japonica, Rickettsia mooseri, Rickettsia prowazekii (Typhus fever), Rickettsia rickettsii, Rickettsia siberica, Rickettsia typhi, Rickettsia conorii, Rickettsia africae, Rickettsia psittaci, Rickettsia quintana, Rickettsia rickettsii, Rickettsia trachomae; Rothia dentocariosa, Salmonella species: Salmonella arizonae, Salmonella Bongori, Salmonella enterica, Salmonella enteriditis, Salmonella paratyphi, Salmonella typhi (Typhoid fever), Salmonella typhimurium, Salmonella salamae, Salmonella arizonae, Salmonella diarizonae, Salmonella houtenae, Salmonella indica; Samsonia species, Serratia species: Serratia entomophila, Serratia ficaria, Serratia fonticola, Serratia grimesii, Serratia liquefaciens, Serratia marcescens, Serratia odoriferae, Serratia plymuthica, Serratia proteamaculans, Serratia quinivorans, Serratia rubidaea, Serratia ureilytica; Shewanella putrefaciens, Shigella boydii, Shigella dysenteriae, Shigella flexneri, Shigella sonnei, Sodalis species, Spirillum species: Spirillum minus rat bite fever, Staphylococcus species: Staphylococcus aureus, Staphylococcus auricularis, Staphylococcus capitis, Staphylococcus caprae, Staphylococcus cohnii, Staphylococcus epidermidis, Staphylococcus felis, Staphylococcus haemolyticus, Staphylococcus hominis, Staphylococcus intermedius, Staphylococcus lugdunensis, Staphylococcus pettenkoferi, Staphylococcus saprophyticus, Staphylococcus schleiferi, Staphylococcus simulans, Staphylococcus vitulus, Staphylococcus warneri, Staphylococcus xylosus; Stenotrophomonas species: Stenotrophomonas acidaminiphila, Stenotrophomonas dokdonensis, Stenotrophomonas koreensis, Stenotrophomonas maltophilia, Stenotrophomonas nitritireducens, Stenotrophomonas rhizophila; Streptobacillus species: Streptobacillus moniliformis (Streptobacillary rat bite fever); Streptococcus species: Streptococcus Group A, Streptococcus Group B, Streptococcus agalactiae, Streptococcus aginosus, Streptococcus avium, Streptococcus bovis, Streptococcus canis, Streptococcus cricetus, Streptococcus faceium, Streptococcus faecalis, Streptococcus ferus, Streptococcus gallinarum, Streptococcus lactis, Streptococcus milleri, Streptococcus mitior, Streptococcus mitis, Streptococcus mutans, Streptococcus oralis, Streptococcus peroris, Streptococcus pneumoniae, Streptococcus pyogenes, Streptococcus ratti, Streptococcus salivarius, Streptococcus sanguinis, Streptococcus sobrinus, Streptococcus parasanguinis, Streptococcus suis, Streptococcus thermophilus, Streptococcus vestibularis, Streptococcus viridans, Streptococcus uberis, Streptococcus zooepidemicus; Tatumella species, Trabulsiella species, Treponema species: Treponema carateum (Pinta), Treponema denticola, Treponema endemicum (Bejel), Treponema pallidum (Syphilis), Treponema pertenue (Yaws); Tropheryma whipplei (Whipple disease), Tuberculoid leprosy, Ureaplasma urealyticum, Veillonella, Vibrio species: Vibrio aerogenes, Vibrio aestuarianus, Vibrio agarivorans, Vibrio albensis, Vibrio alginolyticus, Vibrio brasiliensis, Vibrio calviensis, Vibrio campbellii, Vibrio chagasii, Vibrio cholerae (Cholera), Vibrio cincinnatiensis, Vibrio Comma, Vibrio coralliilyticus, Vibrio crassostreae, Vibrio cyclitrophicus, Vibrio diabolicus, Vibrio diazotrophicus, Vibrio ezurae, Vibrio fischeri, Vibrio fluvialis, Vibrio fortis, Vibrio furnissii, Vibrio gallicus, Vibrio gazogenes, Vibrio gigantis, Vibrio halioticoli, Vibrio harveyi, Vibrio hepatarius, Vibrio hispanicus, Vibrio ichthyoenteri, Vibrio kanaloae, Vibrio lentus, Vibrio litoralis, Vibrio logei, Vibrio mediterranei, Vibrio metschnikovii, Vibrio mimicus, Vibrio mytili, Vibrio natriegens, Vibrio navarrensis, Vibrio neonatus, Vibrio neptunius, Vibrio nereis, Vibrio nigripulchritudo, Vibrio ordalii, Vibrio orientalis, Vibrio pacinii, Vibrio parahaemolyticus, Vibrio pectenicida, Vibrio penaeicida, Vibrio pomeroyi, Vibrio ponticus, Vibrio proteolyticus, Vibrio rotiferianus, Vibrio ruber, Vibrio rumoiensis, Vibrio salmonicida, Vibrio scophthalmi, Vibrio splendidus, Vibrio superstes, Vibrio tapetis, Vibrio tasmaniensis, Vibrio tubiashii, Vibrio vulnificus, Vibrio wodanis, Vibrio xuii; Vogesella indigofera, Wigglesworthia species, Wolbachia species, Xenorhabdus species, Yersinia enterocolitica, Yersinia pestis, Yersinia pseudotuberculosis, and Yokenella species.

Exemplary viruses that can be identified using the present methods, or useful as a Whole Organism Control, include, but are not limited to, Adeno-associated virus, Aichi virus, Australian bat lyssavirus, BK polyomavirus, Baculovirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human herpesvirus 1, Human herpesvirus 2, Human herpesvirus 6, Human herpesvirus 7, Human herpesvirus 8, Human immunodeficiency virus, Human immunodeficiency virus type 1, human immunodeficiency virus type 2, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Parvovirus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus.

Exemplary fungi that can be identified using the present methods, or useful as a Whole Organism Control, include, but are not limited to, Candida species, Aspergillus fumigatus, and Cryptococcus neoformans, non-fumigatus Aspergillus, Zygomycetes, Fusarium and Pseudallescheria boydii.

Exemplary archaea that can be identified using the present methods, or useful as a Whole Organism Control, include, but are not limited to, Euryarchaeota, Crenarchaeota, Nanoarchaeota, Thaumarchaeota, Aigarchaeota, Crenarchaeota, Korarchaeota, Metallosphera sedula, Methanobacterium thermoautotrophicum, Methanococcus jannaschii, Methanococcus jannaschii, Pyrococcus abyssi ST549, Pyrococcus furiosus, Pyrococcus furiosus, Pyrococcus furiosus, Pyrococcus furiosus, Sulfolobus shibatae, Sulfolobus solfataricus Gθ, Sulfolobus solfataricus MT4, Thermococcusbarosii, Thermococcus litoralis.

Exemplary protists that can be identified using the present methods, or useful as a Whole Organism Control, include, but are not limited to, Acanthamoeba, Amoeba proteus, Balamuthia, Entamoeba histolytica, Euglena gracilis, Giardia lambila, Paramecium aurelia, Plasmodium falciparum, Plasmopara viticola, Phytophthora infestans, Phytoplankton species, Pediastrum boryanum, Trypanosoma brucei, Trypanosoma cruzi.

Exemplary parasites that can be identified using the present methods, or useful as a Whole Organism Control, include, but are not limited to, Ectoparasites, Endoparasites, Mesoparasites, Parasitoids, Hyperparasites, Social parasites, Adelpho-parasites.

Exemplary eukaryotes or eukaryotic cells that useful in the present methods, include, but are not limited to, Chinese hamster ovary (CHO, CHO-K1, CHO pro-3, DUKX-X11, DG44), Human embryonic kidney 293 (HEK 293, HEK 293T), Madin Darby canine kidney (MDCK), mouse myeloma cells (NS0, Sp2/0), MEL, African green monkey kidney (COS) cells, insect cells (Spodoptera frugiperda, Sf9, Sf21, Trichoplusia ni), yeast cells (Pichia pastoris, Saccharomyces cerevisiae).

### Methods of Detection

In various embodiments, the disclosure contemplates methods for providing a sample comprising one or more protected nucleic acid sequence, such as substantially intact biological cells and/or viruses, that will provide a desired result when processed using a NAT and can be used to assess the functionality of the NAT. In various embodiments, the method comprises: (a) Modifying at least one protecting vehicle, e.g., a biological cell and/or virus, to include nucleic acids or nucleic acid sequence(s) that were not natively present in said protecting vehicle (b) providing in a liquid (or as a solid for the purpose of reconstitution into a liquid form) the said one or more protecting vehicle, e.g., substantially intact biological cells and/or viruses, comprising both a nucleic acid sequence(s) that were not natively present (a second nucleic acid sequence), and an additional nucleic acid sequence(s) that is natively present (first nucleic acid sequence) of the organism suspected of being the sample to be tested for in the NAT, such that the desired result of the NAT is the detection of nucleic acids of the second nucleic acid sequence and of the first nucleic acid sequence.

In an alternative embodiment, the method comprises: (a) Modifying at least one protecting vehicle, such as a biological cell and/or virus, to include nucleic acids or nucleic acid sequences that were not natively present in the protecting vehicle, (b) providing in a liquid (or as a solid for the purpose of reconstitution into a liquid form) the one or more protecting vehicles, such as substantially intact biological cells and/or viruses, wherein at least one of the protecting vehicles, e.g. cells and or viruses, includes a nucleic acid sequence(s) that were not natively present (second nucleic acid sequence), and wherein either the same protecting vehicle, e.g., cell or virus, or an additional cell or virus, includes a nucleic acid sequence(s) that is natively present in the cell or organism in the sample to be tested for by the NAT (first nucleic acid sequence), such that the desired result of the NAT is the detection of nucleic acids of the first and second nucleic acid sequences.

In various embodiments of the invention, the quality control composition of the invention comprises a protected nucleic acid sequence or sequences, wherein said nucleic acid sequence(s) are alone or together characteristic of a target nucleic acid sequence or the native organism (the organism which does not have the target nucleic acid sequence, and is of the same family), to be tested for in a NAT that are in a protecting vehicle or multiple protecting vehicles. In various embodiments, the said protecting vehicles comprises one or more nucleic acid sequences alone or together which comprises 0.5 to 100% of the target nucleic acid or genome native to the organism, as the case may be, to be tested for in the NAT, or greater than 90%, or greater than 80%, or greater than 70%, or greater than 60%, or greater than 50%, or greater than 40%, or greater than, 30%, or greater than 20%, or greater than 15%, or greater than 10%, or greater than 5%, or greater than 4%, or greater than, 3%, or greater than 2%, or greater than 1%. It should be noted that in some embodiments, the protecting vehicle of the nucleic acid of the native organism, is not the native organism suspected to be in the sample, but can be another protecting vehicle. In various other embodiments, the quality control composition of the invention comprises an organism that is similarly processed in the NAT, but different from the native organism suspected of being in the sample to be tested for the NAT, and it is the native, or genomic sequence of that organism that is being tested for in the NAT as a control for how the system or certain aspects of the system processes said class of organisms.

In various embodiments, the method comprises: (a) Modifying at least one protecting vehicle, such as a biological cell and/or virus, to include nucleic acids or nucleic acid sequence(s) that were not natively present, (b) providing in a liquid (or as a solid for the purpose of reconstitution into a liquid form) the said protecting vehicle, e.g., substantially intact biological cell and/or virus, that includes the nucleic acids that were not natively present (second nucleic acid sequence), and additionally another protecting vehicle, e.g. a substantially intact biological cell and/or virus, comprising naturally occurring nucleic acid sequences (a first nucleic acid sequence), such that the desired result in a NAT is the detection of nucleic acids of the first and second nucleic acid sequences that would ordinarily result if the NAT were performed on a single naturally occurring organism that contained both the first and second nucleic acid sequences.

In other embodiments, the disclosure provides methods for providing a sample comprising protecting vehicle, such as substantially intact biological cells and/or viruses, that will provide a desired result that would not be achieved using said protecting vehicles, such as biological cells and or viruses, in their unmodified state when processed using a NAT, but that might be achieved using a different protecting vehicle, such as a naturally-occurring cell or virus. In various embodiments, the method comprises: (a) Modifying the said protecting vehicles, e.g., biological cells and/or viruses, to include nucleic acids or nucleic acid sequence(s) that were not natively present, (b) providing in a liquid (or as a solid for the purpose of reconstitution into a liquid form) the said protecting vehicles, e.g., substantially intact biological cells and/or viruses, that includes the nucleic acids that were not natively present (second nucleic acid sequence), and the naturally occurring nucleic acid sequences (first nucleic acid sequence), such that the desired result in a NAT is the detection of nucleic acids of the first nucleic acid sequence and from the second sequence that would ordinarily result if the NAT were performed on a single naturally occurring organism that contained both the first and second nucleic acid sequences.

In other embodiments, the disclosure provides methods for providing a test sample comprising more than one protected nucleic acid sequences, such as substantially intact biological cells and/or viruses, that will allow for detection in a NAT. In various embodiments, the method comprises: (a) Modifying at least one of two or more protecting vehicles, e.g., substantially intact biological cells and/or viruses, to include nucleic acids or nucleic acid sequence(s) that were not natively present, (b) providing in combination in a liquid (or as a solid for the purpose of reconstitution into a liquid form) the said protecting vehicle, e.g. substantially intact biological cell and/or virus, that includes the nucleic acids that were not natively present (second nucleic acid sequence), and a protected nucleic acid sequence, e.g. the substantially intact biological cell and/or virus, comprising naturally occurring or native nucleic acid sequences (first nucleic acid sequence), such that the desired result in a NAT is the detection of nucleic acids of the first and second nucleic acid sequences that would ordinarily result if the NAT were performed on a single naturally occurring organism that contained both the first and second nucleic acid sequences.

In various embodiments, the disclosure provides methods for providing a Control for the detection of an organism in a biological test sample using a NAT, wherein the organism is a member of a species or genus that includes members with and without a particular first genetic trait or characteristic that is related to a nucleic acid sequence(s) in the genetic material of some but not all members, and wherein the organism comprises a second genetic trait or characteristic that is not shared by all members of its species or genus, and wherein the NAT detects the genetic trait or characteristic characterized by the first genetic sequence and a second trait or characteristic related to a genetic second sequence that is not common to members of the genus or species. In various embodiments, the method comprises: (a) providing Control material comprising protected nucleic acid sequences, e.g., substantially intact cells and/or viruses, as described herein in a liquid (or as a solid for the purpose of reconstitution into a liquid form) wherein one of the protected nucleic acid sequences, e.g., intact cells or viruses comprises the first genetic sequence and one of the intact cells or viruses comprises the second genetic sequence; (b) detecting the first trait or characteristic using a NAT; and (c) detecting the second trait or characteristic using a NAT, whereby the result of the NAT is similar to the result that would be obtained for a single organism bearing both the first and second trait.

In various embodiments, the disclosure provides methods for providing a Control for the detection of an organism in a biological test sample using a NAT. In various embodiments, the method comprises: (a) obtaining a biological sample suspected of comprising the organism or nucleic acid of interest; (b) providing Control material comprising one or more protecting vehicles, e.g., substantially intact cells and/or viruses, as described herein in a liquid (or as a solid for the purpose of reconstitution into a liquid form); (c) detecting the organism using NAT; and (d) detecting the Control material using NAT. In related embodiments, the protecting vehicles, or substantially intact cell and/or virus or the Control may be a single protecting vehicle or organism or two or more protecting vehicles or organisms (multiorganism). In some embodiments, one protecting vehicle or organism may contain a nucleic acid sequence providing a characteristic not natively present in either protecting vehicle or organism (e.g. a recombinant nucleic acid sequence). In some embodiments, the use of the Control may be used to confirm the proper functioning of the NAT such that the result of the NAT performed on the biological test sample is confirmed (or validated or trusted or supported or Controlled) or otherwise determined to be useful. In some embodiments, the Control material will provide a result in NAT that is positive or confirmatory, in other word that is qualitative. In some embodiments, the Control material will provide a result in NAT that is of a known or expected or consistent or comparable quantity to some result, either prior or concurrent, in other words is quantitative.

In various embodiments, the disclosure provides methods for providing a test sample for the detection of an organism in a biological test sample using a NAT, the method comprising: (a) providing Control material comprising a protected nucleic acid sequence, e.g., substantially intact cells and/or viruses, as described herein in a liquid (or as a solid for the purpose of reconstitution into a liquid form); and (b) detecting the Control material using NAT. In related embodiments, the protected nucleic acid sequence, e.g., substantially intact cell and/or virus of the Control may be within a single protecting vehicle (e.g. organism) or two or more protecting vehicles (e.g., organisms) (multiorganism). In some embodiments, one protecting vehicle, or organism may contain a nucleic acid sequence providing a characteristic not natively present in either protecting vehicle or organism (e.g. a recombinant nucleic acid sequence). In some embodiments, the use of the Control may be used to confirm the proper functioning of the NAT such that the result of the NAT performed on the biological test sample is confirmed (or validated or trusted or supported or Controlled) or otherwise determined to be useful. In some embodiments, the Control material will provide a result in NAT that is positive or confirmatory, in other words that is qualitative. In some embodiments, the Control material will provide a result in NAT that is of a known (or expected or consistent or comparable quantity to some standard) result, either prior or concurrent, in other words is quantitative. In some embodiments, the Control material will be used to test, prove, verify, confirm or otherwise assess the proficiency of the NAT and/or of the laboratory, facility or agency that performs the NAT; in other words the Control material may be used in proficiency testing or interlaboratory comparison, including without limitation government- or industry-mandated proficiency testing programs, accreditation programs, quality assurance programs or similar testing schemes whether required by legislation or standards or not.

In various embodiments, the disclosure provides methods for providing a test sample for the development of or testing of or confirmation of or qualification of a NAT or for training for the purpose of providing and performing a NAT. The method comprising: (a) providing a Control material comprising protected nucleic acid sequences, e.g. substantially intact cells and/or viruses, as described herein in a liquid (or as a solid for the purpose of reconstitution into a liquid form); and (b) detecting the Control material using NAT. In related embodiments, the protected nucleic acid sequences, e.g., substantially intact cell and/or virus, of the Control may be within a single protected vehicle (e.g., organism) or two or more protected vehicles (e.g., organisms) (multi-vehicle or multi organism). In some embodiments, one protected vehicle, e.g., organism, may contain a nucleic acid sequence providing a characteristic not natively present in either protecting vehicle or organism (e.g. a recombinant nucleic acid sequence). In some embodiments, the use of the Control may be to develop or modify or challenge or test the NAT such that the proper functioning of the NAT is assessed for the purpose of providing utility or future utility in the testing of a biological test sample or group or class of test samples. In some embodiments, the Control material will provide a result in NAT that is positive or confirmatory, in other word that is qualitative. In some embodiments, the Control material will provide a result in NAT that is of a known or expected or consistent or comparable quantity to some result, either prior or concurrent, in other words is quantitative. In some embodiments, the Control material will be used to support development of a NAT. In other embodiments, the Control material will be used to support the installation and/or adoption and/or operational qualification of a NAT, for example but not by way of limitation at a site where the NAT was not previously used. In other embodiments, the Control material will be used to support training or qualification of operators and or systems of management or quality assurance in the use of a NAT. In some embodiments, the Control Materials may be referred to as verification and/or validation Controls, or by other related or similar terms.

In various embodiments, the disclosure provides methods for providing a test sample according to the methods described herein wherein the medium in which the test sample is provided (which may be a solid provided for the purpose of reconstitution into a liquid form) includes, but is not limited to, liquids that are, are similar to, or that mimic in some way biological liquids, or that perform similarly to biological medias in respect of their compatibility with the performance of a NAT. Exemplary media include biological liquids such as whole blood, serum, plasma, defibrinated plasma, stabilized plasma pool, cerebrospinal fluid, urine, saliva, semen, sputum, sweat, ocular secretions, nasal secretions, and vaginal secretions. In various embodiments, the test sample may be provided in or on matrices or containers or surfaces in which form biological samples may be provided, including blood collection tubes, other sample collection tubes, preservative liquids associated with collection devices, swabs, papers, or other collection, preservation, or transport vessels or media.

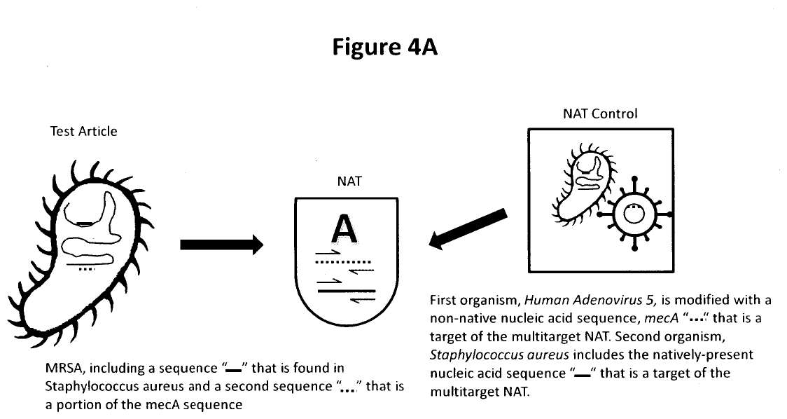

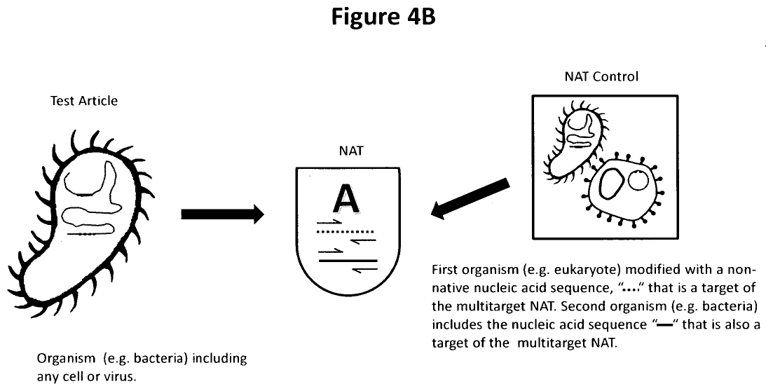

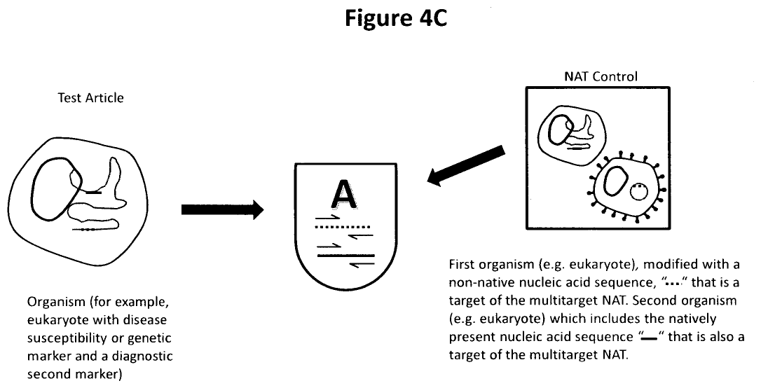

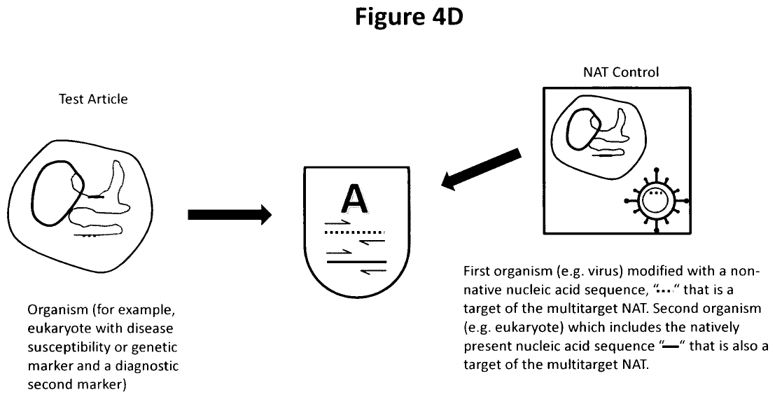

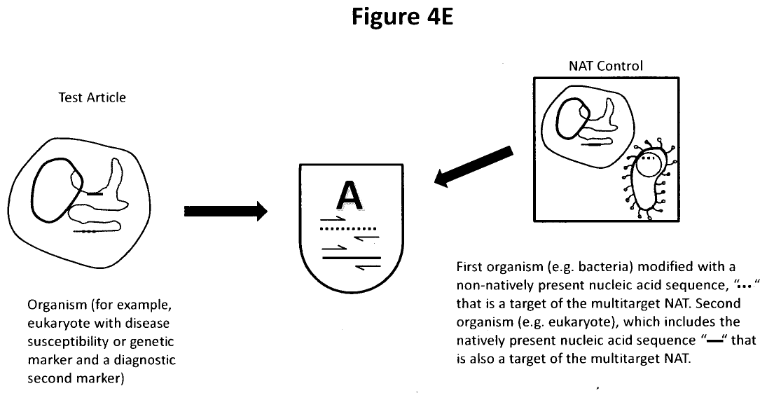

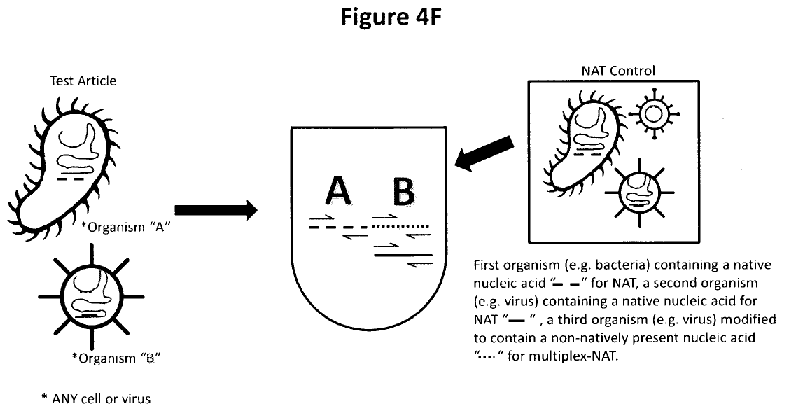

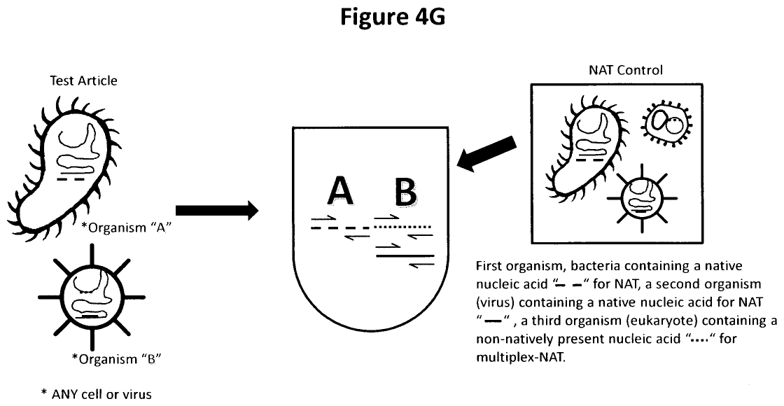

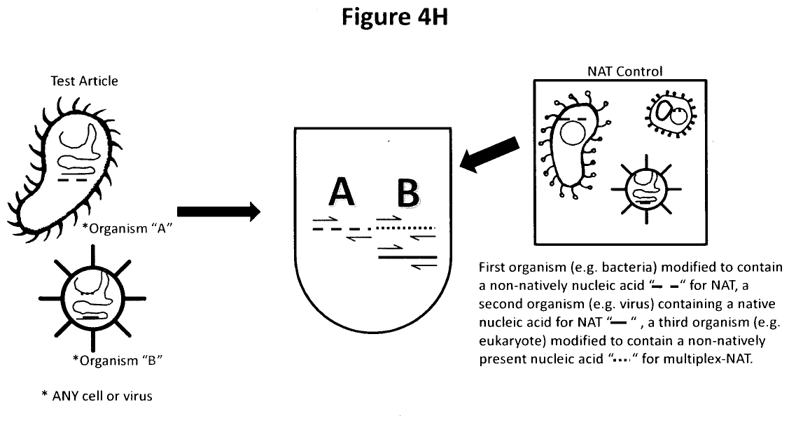

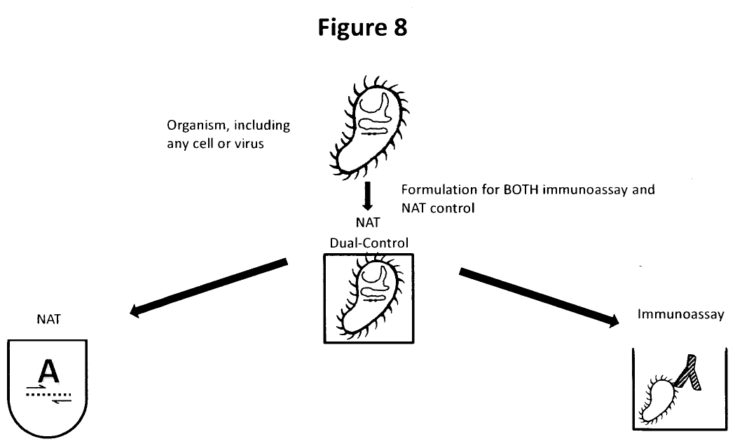

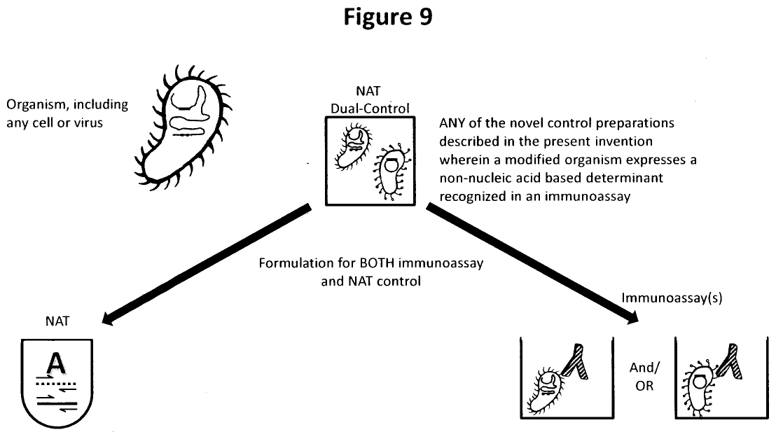

In various embodiments, the invention provides a quality control composition for assessing the functionality of a NAT and optionally more than one NAT and/or additional detection systems (such as nucleic acid, non-nucleic acid detection systems/tests). As such, in some aspects, the invention comprises any one of the compositions of the invention wherein the NAT is used to detect the presence of one or more additional organisms in a test article, the composition further comprises one or more additional controls for the same or different organism or organisms or target nucleic acid sequence or sequences of the NAT, each of them being tested for in the NAT. For example, see FIG. 4F, 4G, 4H, 5C or 6C, 10, which depict a multiplex NAT, wherein the quality control composition comprises the additional quality control components for the second organism in the test article organism “A” in addition to the quality control components for organism “B”. Alternatively, or in addition, the quality control composition of the present invention may comprise a non-NAT based detection system of additional organisms that may be suspected of being in the sample or test article. Also, in addition or alternatively, the quality control composition of the present invention may comprise further control components for the same organism (dual or multi-control), such as an additional quality control materials for a NAT with a different target sequence for the same organism suspected of being in the sample (or test article) or a different nucleic acid test (non-NAT), or for a non-nucleic acid based detection system, e.g. immunoassay, and use of antibodies (e.g. see FIG. 8 or 9). As such, the invention also provides methods for conducting the control test (s) using said compositions, wherein the NAT is run simultaneously, in parallel or separately from (i) running the same tests for the sample or test article; or (ii) simultaneously, in parallel or separately from each other (i.e. the other test, whether it be an additional NAT or another detection system).

In various embodiments, the control may comprise two or more different organisms combined and evaluated by two or more NAT, where the combination of results from the two or more NAT may be obtained from a single naturally-occurring organism that naturally comprises sequences recognized by the two or more NAT. In yet other embodiments, the two or more different organisms give a result in the combination of the two or more NAT tests that is not distinguished from the result that may be obtained from a single naturally occurring organism, where the result from the two or more organisms combined may be used as a Control for the NAT proper functioning of the NAT such that the NAT are confirmed or validated or trusted or supported or Controlled or otherwise determined to be useful.

In various embodiments, the control may comprise two or more different organisms combined and evaluated by two or more NAT, where the combination of results from the two or more NAT may be obtained from a single naturally occurring organism that naturally comprises sequences recognized by the two or more NAT, and wherein the single naturally occurring organism may be more hazardous and/or biohazardous in its viable state. In yet other embodiments, the test sample may be produced at a lower biosafety level or at a lower level of risk to individuals involved in its production and/or use than the naturally occurring organism.

In various embodiments, the controls of the invention comprise sequences recognized by a NAT that are similar to sequences found in a naturally occurring organism, wherein those sequences are modified from the naturally occurring sequences. For example and not by way of limitation, the sequences may be truncated, or may be altered by changes to the sequence, or altered by posttranslational modification of some nucleic acids. By way of further example and not limitation, the sequences may incorporate changes to alter the transcription or translation of genes, such as the introduction of stop codons, rare codons, changes to the amino acids for which the sequence codes, changes to genetic elements controlling, promoting, or inhibiting transcription or translation, or other changes. It will be recognized by one skilled in the art that it is possible to introduce changes in a genetic sequence that do not abrogate its recognition in a NAT designed for similar sequence. Further, it will be recognized that changes either in portions of the sequence that interact with components of the NAT (for example primers and probes in PCR-based tests) and parts of these sequence that do not, can, in many cases, be made without abrogating recognition in such tests.

In various embodiments, the disclosure provides methods for providing a Control for the detection of an organism in a biological test sample using an Immunoassay or an Expression-based assay. In various embodiments, the method comprises: (a) obtaining a biological sample suspected of comprising the organism or nucleic acid of interest; (b) providing Control material comprising materials as described herein in a liquid (or as a solid for the purpose of reconstitution into a liquid form); (c) detecting the organism using an Immunoassay or an Expression-based assay; and (d) detecting the Control material using an Immunoassay or an Expression-based assay.

Notwithstanding the use of multiple organisms or the potential of more than one organism to behave like a single organism in the context of a NAT or Immunoassay or other test, nevertheless the current disclosure contemplates in some aspects the utility of the test materials in Controls where several organisms are represented; in some cases, not all of the test materials included in such multi-organism need be made by the methods provided herein, and those that are not made by these methods are not contemplated under this disclosure except that they may be compatible and may be included in a Multi-Organism Control along with the test materials constructed as provided in this disclosure. For example, and not by way of limitation, a Control material might be provided for a NAT capable of detecting both a naturally occurring organism ‘A’ and a naturally occurring organism ‘B’, wherein organism ‘A’ is a drug-resistant organism. The portion of the Control recognized by the NAT to detect organism ‘A’ may be constructed as described herein from one or more organisms containing at least one non-native sequence recognized by the NAT, while the portion of the Control material recognized by the NAT to detect organism ‘B’ may be constructed by methods outside the present disclosure.

This disclosure contemplates methods for constructing Whole Organism Controls wherein a key advantage of the use of such Controls is that Whole Organism Controls can be subjected to the same whole process as naturally-obtained biological samples, including but not limited to steps where samples may be concentrated and/or nucleic acids purified. However, it is also contemplated herein that Controls useful in NAT can be constructed wherein free nucleic acids (outside of Whole Organism Controls) may be added to a Control preparation in sufficiently large quantity to survive concentration and/or purification procedures that would ordinarily remove small amounts of free nucleic acid. The present disclosure provides that quantities of free nucleic acids added to a Whole Organism Control can substitute for a second whole organism that carries a sequence not native or endogenous in the first organism that is recognized in a NAT.

### Kit

Provided herein is a kit, comprising quality control composition or components of same, useful for assessing the functionality of a NAT. In some aspects of the invention, the kit comprises Whole Organism Controls or Multi-Organism Controls, useful for detecting an organism or nucleic acid in a biological test sample using NAT. The kit may further comprise instructions for using the kit and conducting the NAT using the quality control compositions and/or components thereof, e.g., Whole Organism or Multi-Organism Controls, and their formulation or composition.

The embodiments described below illustrate representative examples of methods of the disclosure. From the description of these embodiments, other aspects of the invention can be made and/or practiced based on the description provided below. The methods involve use of immunological and molecular biological techniques described in methodology treatises such as Current Protocols in Immunology, Coligan et al., ed., John Wiley & Sons, New York. Techniques of molecular biology are described in detail in treatises such as Molecular Cloning: A Laboratory Manual, 2nd ed., vol. 1-3, Sambrook et al., ed., Cold Spring Harbor Laboratory Press, Cold Spring Harbor, N.Y., 2001; and Current Protocols in Molecular Biology, Ausubel et al., ed., Greene Publishing and Wiley-Interscience, New York. General methods of medical treatment are described in McPhee and Papadakis, Current Medical Diagnosis and Treatment 2010, 49th Edition, McGraw-Hill Medical, 2010; and Fauci et al., Harrison's Principles of Internal Medicine, 17th Edition, McGraw-Hill Professional, 2008.

### EXAMPLES

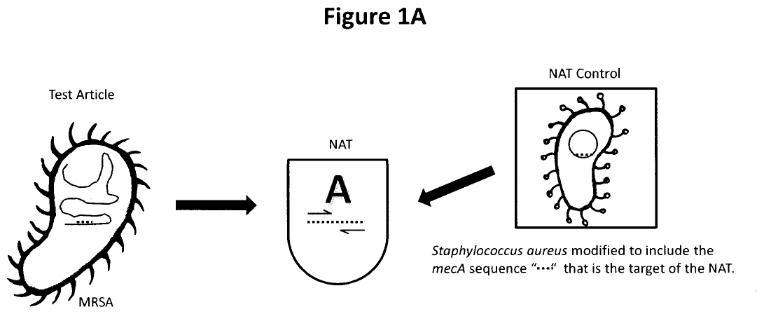

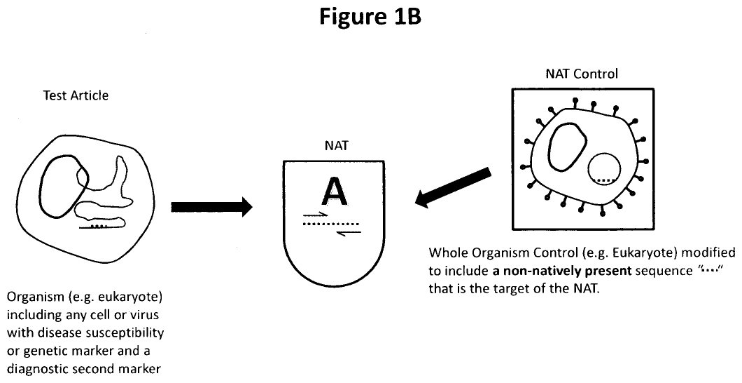

The following Examples are merely illustrative and are not intended to limit the scope or content of the invention in any way. FIGS. 1B, 2B, 4B-4H, 5B-5C and 6B-6C illustrate variations on the methods discussed in the Detailed Description and Examples below.

### Example 1: Quality Control Composition (Augmented Whole Organism Controls) Useful for Assessing the Functionality of a NAT for Detecting MRSA

Staphylococcus aureus strains, including those resistant to methicillin and many other antibiotics, are major causes of nosocomial infections worldwide (Diekema et al., Clin Infect Dis. 2001; 32:S114-32). Resistance to methicillin is determined by the mecA gene, which encodes the low-affinity penicillin-binding protein PBP 2A (Beck et al., J Bacteriol. 1986; 165(2):373-8). A single Augmented Molecular Control comprising a Whole Organism Control is used for a NAT to detect the presence of Methicillin-resistant Staphylococcus aureus (MRSA).

As an example, the Whole Organism Control comprises Staphylococcus Aureus containing a nucleic acid providing a characteristic that is not natively present to strains of Staphylococcus Aureus that are not resistant to methicillin, such as the mecA gene. Staphylococcus Aureus is genetically modified to contain the mecA gene or a portion thereof by transfection with a plasmid or vector into which the mecA gene has been engineered using routine molecular biological methods know to those skilled in the art (FIG. 1A). The modified Whole Organism Control is subsequently used as an Augmented Molecular Control in a NAT. Direct genetic modification of Staphylococcus aureus is achievable by any of a large number of means including those described herein, and may equally be achieved by new methods that will be developed for the purpose, the genetically modified products of which are contemplated for use in the construction of controls as described herein.

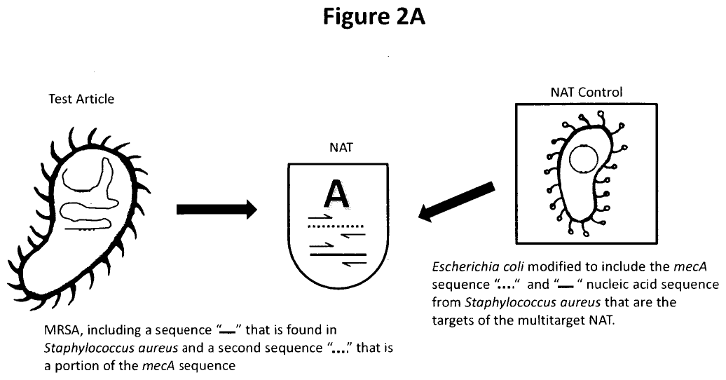

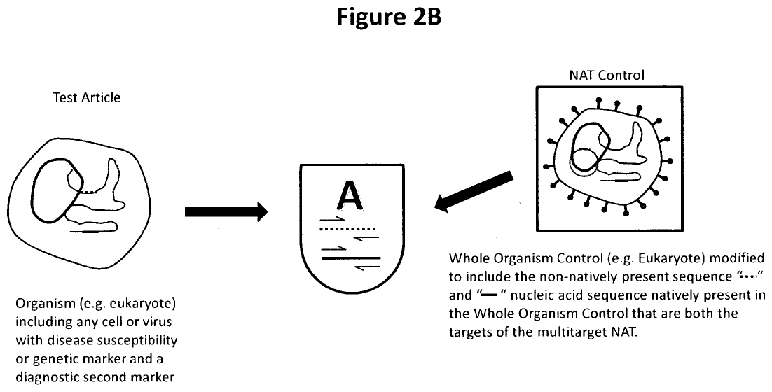

Alternatively, the Whole Organism Control comprises Escherichia coli (E. coli) containing two nucleic acids providing one or more characteristics that are not natively present in E. coli. The E. coli is genetically modified to contain both an exogenous nucleic acid from Staphylococcus aureus and the mecA gene or a portion thereof by transfection with one or more plasmids or vectors into which the nucleic acid from Staphylococcus aureus and mecA gene have been engineered using routine molecular biological methods know to those skilled in the art. The modified Whole Organism Control is subsequently used as an Augmented Molecular Control in a multitarget NAT (FIG. 2A).

Alternatively, the Whole Organism Control comprises Staphylococcus Aureus containing a nucleic acid providing a characteristic that is not natively present to strains of Staphylococcus Aureus that are not resistant to methicillin, such as the mecA gene. The Whole Organism Control further comprises E. coli genetically modified to contain the mecA gene or a portion thereof by transfection with a plasmid or vector into which the mecA gene has been engineered using routine molecular biological methods know to those skilled in the art. The Whole Organism Control is subsequently used as a Control in a NAT, including but not limited to NAT that detect Staphylococcus Aureus, NAT that detect mecA, and/or NAT that detect MRSA. The Control is broadly useful for NAT designed by various manufacturers and designers of NAT since it can be constructed to comprise many or most or all of the sequences typical of MRSA and/or of mecA and/or of Staphylococcus Aureus. The invention so described provides a novel and useful solution to the construction of broadly applicable (i.e. across many diverse NAT related to MRSA) controls that is fast, can flexibly be applied to the construction of controls, and that permits the construction and manufacturing and delivery of controls for a biohazardous organism (MRSA) in a way that permits much safer production and handling because none of the components of the control is as hazardous as MRSA.

In each case, the Whole Organism Control is constructed to produce a positive signal in a NAT designed to detect MRSA, comprising a component NAT for the presence of Staphylococcus aureus (first nucleic acid sequence) and a component NAT for the presence of mecA (second nucleic acid sequence). The sample is determined to contain Staphylococcus aureus and mecA, and/or is determined to indicate the presence of MRSA.

In some other aspects of the present invention, another highly useful feature of the invention is that sequences that might be related by similarity to each other, but might be found in different species of organisms, can be quickly assembled with vehicles, protected vehicles or whole organisms or combinations of same to produce controls for different NAT or in some preferred and useful embodiments, from at least one protected vehicle and/or whole organism or in some other preferred embodiments, the control of the invention comprising at least one organism. For example, but not by way of limitation, the mutS-rpoS locus of E. coli O157:H7 is shared in near identity with the mutS-rpoS locus of S. dysenteriae (LeClerc et al., Journal of Bacteriology, December 1999, p. 7614-7617). It will generally be appreciated that the mutS-rpoS locus can be isolated in a plasmid and further can be rendered as unharmful nucleic acid sequence by methods known to those skilled in the art and discussed in part elsewhere herein, and that such a plasmid can be purified and placed in a protective vehicle, for example a liposome. The method of the present invention teaches that the admixture of such a protected plasmid and an ordinary, non-pathogenic E. coli organism may be performed to provide a control for pathogenic E. coli O157:H7 in NAT that detect sequences both of E. coli and of mutS-rpoS. Furthermore, in some other aspects, the invention teaches that the same liposome-protected plasmid can be mixed with (for example) bacterial strain ECOR24, which is a very close relative of Shigella species without the pathogenicity of most such species (Pupo et al., Infection and Immunity, July 1997, p. 2685-2692), to provide a control in NAT that detect sequences both of Shigella and mutS-rpoS. In this way, the manufacture of controls for NAT will be simplified because one protected nucleic acid (mutS-rpoS protected in a liposome) can be used in the manufacture of two controls, both simplifying and speeding the development and delivery of such controls. Furthermore, in the example given, the controls so produced are useful for pathogenic organisms but can be made and delivered without the complication of manufacturing hazardous materials.

It will readily be apparent that the teaching of the present invention is generalizable to the construction of controls for other circumstances where more than one sequence is detected by a NAT, so that multi-organism controls can provide said sequences in separate protecting vehicles, with advantages of ease of construction, multi-use potential for individual protected sequences once constructed, and safety enhancements for both the providers of such controls, and potentially for the users.

### Example 2: Quality Control Composition (Augmented Multi Organism Controls) Useful for Assessing the Functionality of a NAT for Detecting MRSA

A Multi-Organism Molecular Control comprising an admixture of different organisms is used as a Whole Organism Control for NAT to detect the presence of MRSA.

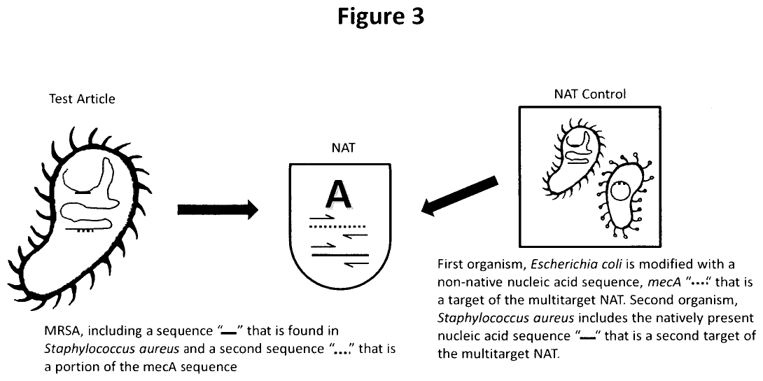

In one example, the Whole Organism Control comprises Staphylococcus Aureus organism admixed with E. coli genetically modified to contain the mecA gene or a portion thereof by transfection with a plasmid or vector into which the whole or partial mecA gene has been engineered by any of a number of methods for construction of recombinant plasmids that are well known to those skilled in the art (FIG. 3). Alternately, the whole or partial mecA gene may be introduced into the E. coli by transduction with an appropriately engineered viral vector, for example a bacteriophage, into which the whole or partial mecA gene has been engineered by any of a number of methods for construction of recombinant viral vectors that are well known to those skilled in the art. Other methods exist for the introduction of genetic material into E. coli that are known, and methods yet unknown may be developed for the introduction of foreign genetic material into E. coli, any of which could be used for constructing a modified E. coli organism for use as a component of a Whole Organism Control according to the teachings of the present disclosure.

The Whole Organism Control is constructed to produce a positive signal in a multitarget NAT designed to detect MRSA, comprising a component NAT for the presence of Staphylococcus aureus (first nucleic acid sequence) and a component NAT for the presence of mecA (second nucleic acid sequence).

Depending on the specific limits incorporated into the multiplex NAT, the sample is determined to contain Staphylococcus aureus and mecA, and/or is determined to indicate the presence of MRSA. The method is not limited to two organisms or traits, but may be extended to the admixture of more organisms or traits as required for the Control for the NAT.

### Example 3: Quality Control Composition (Augmented Multi-Organism Control) Useful for Assessing the Functionality of a NAT for Detecting MRSA

A Multi-Organism Molecular Control comprising an admixture of different organisms, e.g., from different species or origin, is used as a Whole Organism Control Material for NAT to detect the presence of MRSA.

In one example, the Whole Organism Control Material comprises Staphylococcus Aureus organisms admixed with Human adenovirus type 5 genetically modified to contain the mecA gene or a portion thereof engineered by any of a number of methods for construction of recombinant adenoviruses that are well known to those skilled in the art (FIG. 4A). Other viruses exist into which genetic material can be introduced by methods well known to those skilled in the art, including but not limited to adeno-associated viruses, human herpersvirus 1 and human herpesvirus 2 (also known as herpes simplex virus 1 and herpes simplex virus 2), baculovirus, potatovirus X, tobacco mosaic virus, vaccina virus, fowlpox virus, human cytomegalovirus, entomopoxvirus, alphavirus, vesicular stomatitis virus, and many other viruses that can be genetically modified by the introduction of foreign genetic material by methods known or develop in the art, any of which could be used for constructing a modified virus for use as a component of a Whole Organism Control according to the teachings of the present disclosure.

The Whole Organism Control Materials are constructed to produce a positive signal in a multitarget NAT designed to detect MRSA, comprising a component NAT for the presence of Staphylococcus aureus (first nucleic acid sequence) and a component NAT for the presence of mecA (second nucleic acid).

Depending on the specific limits incorporated into the multiplex NAT, the sample is determined to contain Staphylococcus aureus and mecA, and/or is determined to indicate the presence of MRSA. The method is not limited to two organisms or characteristics, but may be extended to the admixture of more organisms or characteristics as required for the Control Materials for the NAT. FIGS. 4B-4H illustrate variations on methods using a multiorganism control.

### Example 4: Quality Control Composition (Augmented Whole Organism Controls) Useful for Assessing the Functionality of a NAT for Detecting MRSA

A Whole Molecular Control comprising an admixture of an organism and a non-organism associated nucleic acid is used as a Whole Organism Control Material for NAT to detect the presence of MRSA.

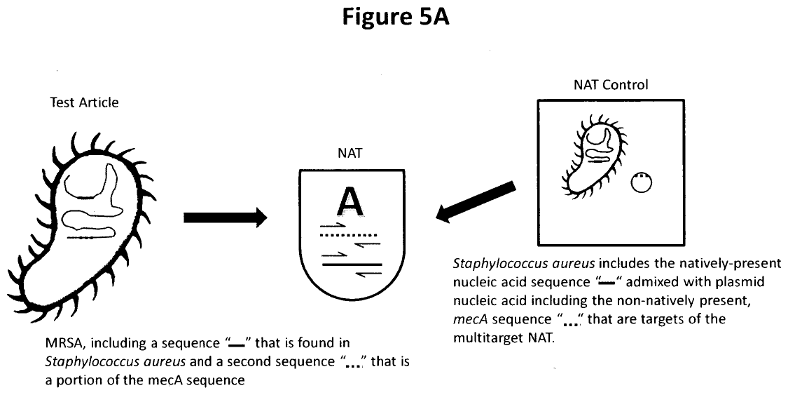

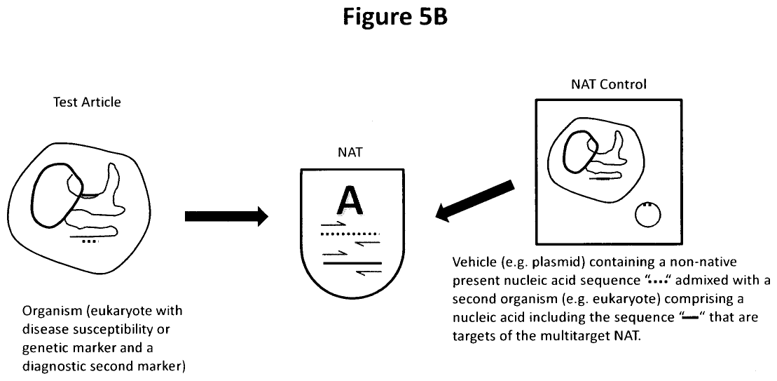

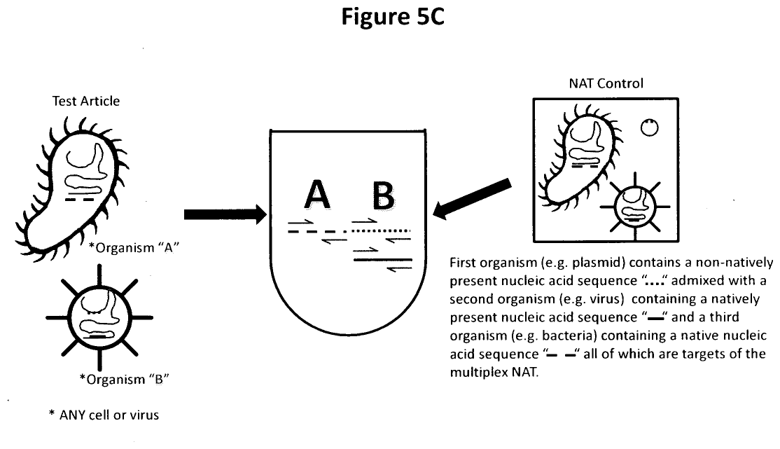

In one example, the Whole Organism Control Material comprises Staphylococcus Aureus organisms admixed with purified plasmid nucleic acid that has been modified to include the mecA gene or a portion thereof by any of a number of methods for construction of recombinant plasmids that are well known to those skilled in the art (FIG. 5A). In one example, the plasmid may be derived from E. coli, but it is contemplated that any other bacterial strains in which modified or recombinant nucleic acid plasmids or constructs can be produced and/or propagated including methods not yet conceived by which the method of the present disclosure are useful in the method. FIG. 5B-5C illustrate variations on methods using a multiorganism control as described above.

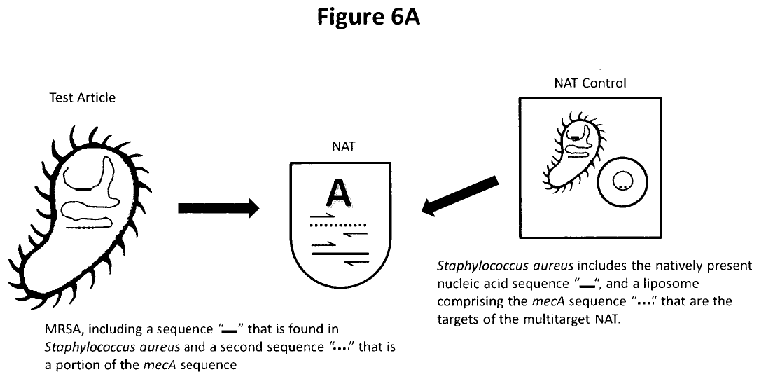

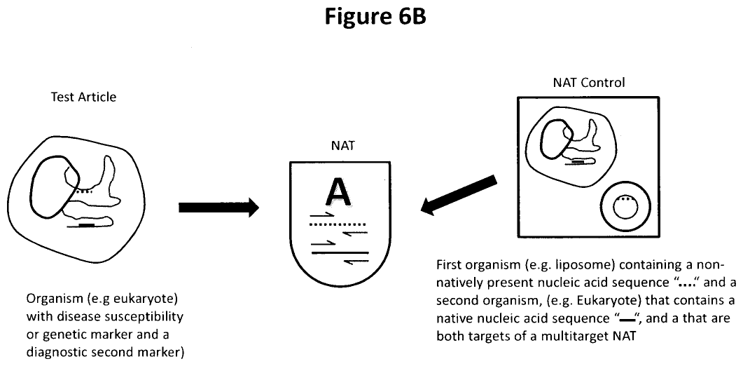

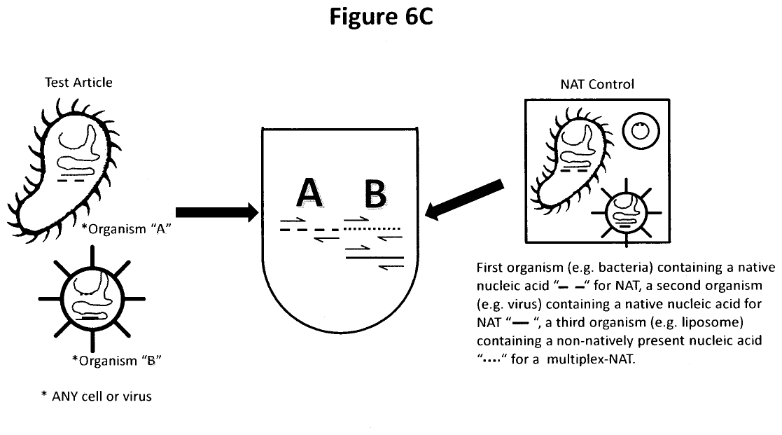

Alternately, the Whole Organism Control Material comprises a first organism, e.g., Staphylococcus aureus, and a second organism, e.g., a liposome, that is engineered to contain a whole or partial mecA gene (FIG. 6A). The Whole Organism Control is constructed to produce a positive signal in a multitarget NAT designed to detect MRSA, comprising a component NAT for the presence of Staphylococcus aureus (first nucleic acid) and a component NAT for the presence of mecA (second nucleic acid). FIGS. 6B-6C illustrate variations on methods using a multiorganism control as described above.

Other methods exist for providing extra-organismal genetic material bearing a genetic sequence including all or part of the mecA gene that are well known to those skilled in the art, including but not limited to isolation from appropriately modified recombinant or natural viruses, isolation from appropriately modified eukaryotic or fungal or archaeal cells, or chemical or biochemical synthesis, and after developed techniques, are contemplated for providing a desired nucleic acid sequence as part of an Augmented Organism Control according to the teachings of the present disclosure. In other embodiments, the Whole Organism Control Material comprises Staphylococcus Aureus organisms admixed with nucleic acids derived from such sources that have been modified include the mecA gene or a portion thereof.

In some variations on the methods herein, the added nucleic acid is provided free or soluble or without further derivation or physical construction.

In other embodiments, the added nucleic acid is provided with some additional modification, such as attachment to or association with a physical particle or containment within a particle. By way of example, but not by limitation, a particle may be plastic, glass, metal, ceramic, a polymer compound, a lipid, a protein, or a particle comprised of another material or of a combination of materials. The attachment may be achieved through chemical means or through noncovalent means. In yet other embodiments, the added nucleic acid is provided within or throughout some non-organismal container or matrix, including, but not limited to, a liposome or nanocarrier, gelatin, sodium alginate, ethyl cellulose, polyvinyl alcohol, or other substance or container capable of containing nucleic acids. In other embodiments, the nucleic acid may be modified chemically, for example by methylation or by association with nucleic-acid binding proteins, or by the introduction of phosphorothioate bonds, or 2′-O-methylation, or the addition of 2′-fluoro bases, or inverted dT, or phosphorylation, or any other modification. It will be understood by those skilled in the art that one advantage of modifying nucleic acids according to the methods described herein (or by methods of similar effect known or related methods not yet known) is to stabilize the nucleic acid for its recognition in a NAT, or to provide it in a form more amenable to retention through the sample processing steps that may form part of an overall procedure or process or test that incorporates a NAT.

The Augmented Whole Organism Control Materials are constructed to produce a positive signal in a multitarget NAT designed to detect MRSA, comprising a component NAT for the presence of Staphylococcus aureus (first nucleic acid) and a component NAT for the presence of mecA (second nucleic acid).

Depending on the specific limits incorporated into the multiplex NAT, the sample is determined to contain Staphylococcus aureus and mecA, and/or is determined to indicate the presence of MRSA. The method is not limited to two organisms or traits, but may be extended to the admixture of more organisms or traits as required for the Control Materials for the NAT.

Notwithstanding the particulars of the exemplary embodiments described above, it will be understood that a Molecular Control for MRSA can be constructed by the addition of materials other than bacteria, or viruses, or free nucleic acid to unmodified Staphylococcus aureus. For instance, genetically modified eukaryotic cells, or archaea, or fungi genetically modified to contain the mecA gene or a portion thereof may be added to Staphylococcus aureus to create a Molecular Control according to the method of the present disclosure.

### Example 5: Quality Control Compositions (Augmented Whole Organism Controls) in Human Influenza Serotyping

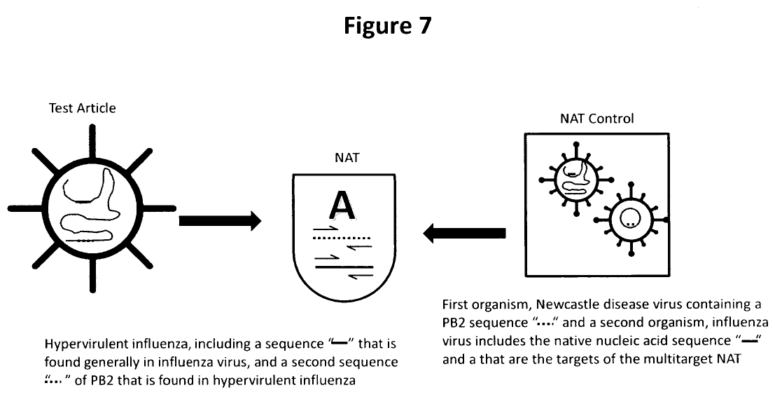

Hyper-virulent and pandemic strains of human influenza virus have been described. These strains may not be available, or handleable, to the developer of a NAT for such a disease, for example if the developer of a NAT is unable to safely manipulate such biological materials under biosafety rules and regulations. In this case an influenza virus preparation of a virus having reduced virulence but is admixed with a recombinant virus, e.g., Newcastle disease virus, that contains an exogenous virulence-encoding influenza gene, or part thereof, for example derived from a sequence encoding a virulent form of protein PB2 (FIG. 7). Using a multiplex NAT designed to detect virulent influenza, the Control Materials are constructed to produce a positive signal in a NAT for the presence of influenza (first nucleic acid) and a positive signal for the presence of virulence-encoding PB2 sequence (second nucleic acid).

Many additional variations in the method are contemplated. For example, the NAT may be performed in the presence of whole or partially disrupted influenza. The virulence sequence may be provided in any useful viral vector, or in non-viral vector systems, or whole plasmids or as a naked nucleotide. Different virulence factors may be analyzed in a different NAT.

In further variations, any number of virulent human viruses may be evaluated in a NAT according to this method.

In yet further variations, non-viral pathogens may be evaluated in a NAT according to this disclosure.

### Example 6: Quality Control Compositions (Augmented Whole Organism Controls) for Animal Heath Applications

Virulent strains of animal pathogens have been described. These strains may not be available, or handleable, to the developer of a NAT for specific animal diseases, for example if the developer of a NAT is unable to safely manipulate such biological materials under biosafety rules and regulations. In this case an avirulent strain of a given pathogen may be provided as a NAT Control in combination with exogenously provided nucleotide sequences that correspond to the pathogen virulence factors.

In one example, avirulent bovine viral diarrhea virus (BVDV) is admixed with a recombinant baculovirus that contains an exogenous BVDV virulence-encoding cleaved NS23 gene. Using a multiplex NAT designed to detect virulent BVDV, the Control Materials are constructed to produce a positive signal in NAT for the presence of BVDV (first nucleic acid) and a positive signal for the presence of cleaved NS23 (second nucleic acid).

Additional variations in the method are contemplated. For example, NAT may be performed in the presence of whole or partially disrupted BVDV. Cleaved NS23 gene may in other embodiments be provided in viral vectors other than baculovirus, or in non-viral vector systems, or whole plasmids or as naked nucleotide.

In further embodiments, any virulent virus, such as Sendia virus, Foot-and-mouth disease virus (FMDV), Eastern equine encephalitis (EEE) or chicken influenza may be evaluated in a NAT according to this invention.

In yet further embodiments, non-viral pathogens may be evaluated in a NAT according to this invention.

In other embodiments, human, as well as animal pathogens may be evaluated in a NAT according to this invention.

### Example 7: Quality Control Compositions (Augmented Whole Organism Controls) for Assessing the Functionality of a NAT for Key Disease Genes

Among many genetically determined diseases of humans is cystic fibrosis, which is determined by one of a number of variants of the cystic fibrosis transmembrane conductance regulator (CFTR) gene. A single Augmented Molecular Control comprising a Whole Organism Control is used for a NAT to detect the presence of CFTR in human cells.

As an example, the Whole Organism Control comprises MRC-5 cells containing a nucleic acid providing a characteristic that is not natively present to MRC-5 such as a variant of the CFTR gene. MRC-5 is genetically modified to contain the variant CFTR gene or a portion thereof by CRISPR-Cas9, viral transduction, or using other routine molecular biological methods know to those skilled in the art. The modified Whole Organism Control is subsequently used as an Augmented Molecular Control in a NAT. Direct genetic modification of MRC-5 is achievable by any of a large number of means including those described herein, and may equally be achieved by new methods that will be developed for the purpose, the genetically modified products of which are contemplated for use in the construction of controls as described herein.

MRC-5 is not an immortal cell line, and in some cases the genetic modification and/or propagation and preservation of immortalized cell lines may be advantageously achieved using immortalized cell lines. Any human cell line appropriate to the purpose may be substituted for MRC-5, whether immortalized or not.

Alternatively, the Whole Organism Control comprises a non-human cell such as a yeast cell (for example Saccharomyces cerevisiae) containing two nucleic acid sequences providing one or more characteristics that are not natively present in the non-human cell. Said non-human cell is genetically modified to contain both an exogenous nucleic acid sequence from the human genome, and a variant CFTR gene or a portion thereof using molecular biological methods know to those skilled in the art. The modified Whole Organism Control is subsequently used as an Augmented Molecular Control in a multitarget NAT that normally detects both sequences to determine the presence of the CFTR gene and the presence of a second human genetic sequence (a multitarget NAT for CFTR).

Using the methods described herein, one skilled in the art will understand that it is possible to construct a Multi-Organism control for a multitarget NAT for CFTR, wherein one organism is a human cell or other organism comprising a sequence detected in the NAT, which may be detected as a component of the NAT for confirming the presence of human DNA, and a second organism is a human cell or other organism comprising a portion of a nucleic acid sequence from a variant of the CFTR gene.

In each case described above, the Whole Organism Control is constructed to produce a positive signal in a NAT designed to detect CFTR, comprising a component NAT for the presence of human cells (first nucleic acid sequence) and a component NAT for the presence of variant CFTR (second nucleic acid sequence). The sample is determined to contain Human cells and CFTR, and/or is determined to indicate the presence of CFTR in a human cell.

### Example 8: Quality Control Compositions for NAT in Combination: Multifunctional Augmented Whole-Organism Controls for NAT and Immunoassay

Hyper-virulent strains of animal pathogens have been described. These strains may not be available, or handleable, to the developer of assays for specific animal diseases, for example if the developer of a NAT is unable to safely manipulate such biological materials under biosafety rules and regulations. They may also be unavailable, or not useful in immunoassay format. In this case an avirulent strain of a given pathogen may be provided as a Control in combination with exogenously provided and expressed nucleotide sequences that correspond to the pathogen virulence factors. Pathogens without such hypervirulence factors may be designated normovirulent.

In one embodiment, a normovirulent virus comprising a nucleic acid sequence ‘X’ is admixed with a recombinant bacterium containing an expressed element ‘Y’ of a virulence-factor encoding gene. Using a multiplex NAT designed to detect the virus and the presence of the virulence factor or part thereof, the Control Materials are constructed to produce a positive signal in NAT for the presence of X (first nucleic acid) and a positive signal for the presence of Y (second nucleic acid). Using the same Whole Organism Control, a contemporaneous immunoassay can be run to detect the expressed protein product of Y (FIGS. 8 and 9).

Additional variations in the method are contemplated. For example, NAT may be performed in the presence of whole or partially disrupted X. The Y gene may in other embodiments be provided in numerous types of vectors other than a bacterium, or as naked nucleotide.

In another variation, a Whole Organism Control comprising a single whole organism and single formulation expressing at least one nucleic acid sequence providing a characteristic of that organism may be tested for use in NAT and immunoassay.

In further variations on the methods, any virulent virus or non-viral pathogens may be evaluated according to this invention.

In other variations on the method, human, as well as animal pathogens may be evaluated according to this invention.

In other embodiments, other expression-based testing, including, but not limited to. Precipitation by DNA aptamer technology may replace Immunoassay according to this invention.

While the foregoing invention has been described in some detail for purposes of clarity and understanding, it will be appreciated by one skilled in the art, from a reading of the disclosure, that various modifications, variations and changes in form and detail can be made without departing from the true scope of the invention in the appended claims.

All publications, patents, and patent applications are herein incorporated by reference in their entirety to the same extent as if each individual publication, patent or patent application was specifically and individually indicated to be incorporated by reference in its entirety.

What is claimed:

1. A quality control composition used to assess the functionality of a Nucleic Acid Test (NAT), wherein the NAT is used to detect the presence of an organism and a target nucleic acid sequence in a test article, the quality control composition comprising:
(a) a first protected nucleic acid sequence comprising a first nucleic acid sequence the presence of which is characteristic of the presence of the organism in the test article said first nucleic acid sequence being tested in the NAT; and
(b) a second protected nucleic acid sequence comprising a second nucleic acid sequence the presence of which is characteristic of the presence of the target nucleic acid sequence in the test article said second nucleic acid sequence being tested for in the NAT,
wherein the first and second protected nucleic acid sequences are together in one or separately in more than one protecting vehicle,
wherein the protecting vehicles can be the same or different type of protecting vehicle,
wherein the second protected nucleic acid sequence is not natively present in its protecting vehicle.

2. The composition of claim 1, wherein the protecting vehicles protect their respective nucleic acid sequences so that the nucleic acid sequences can be substantially recovered in the nucleic acid extraction steps of the NAT.

3. The composition of claim 1 or 2, wherein at least one of the protecting vehicles is a vehicle that has the same or similar nucleic acid protection properties when processed through the NAT as the vehicle the presence of which is being tested for in the test sample.

4. The composition of any one of claims 1 to 3 wherein at least one of the protecting vehicles is an organism that has the same or similar nucleic acid protection properties when processed through the NAT as the organism the presence of which is being tested for in the test sample.

5. The composition of any one of claims 1-4, wherein the protecting vehicle is any enclosing or associating substance or barrier.

6. The composition of any one of claims 1-5, wherein the protection of the nucleic acid sequences may be mediated by their containment within or their association with the protecting vehicle.

7. The composition of any one of claims 1-6 wherein the protecting vehicle of the first and second protected nucleic acid sequences are independently selected from the group consisting of: a virus, a virus-like particle, a bacterium, a eukaryotic cell, an anucleated cell, a liposome, a vesicle, a protein capsid or shell, a microcompartment, a nanovesicle, an exosome, a micelle, a solid lipid nanoparticle, a lipid-coated particle, a nanotube, a nanocrystal, a polymeric nanoparticle, an inorganic nanoparticle, an interpolyelectrolyte complex (polyplex), or a dendrimer.

8. The composition of any one of claims 1-7, wherein the protecting vehicle of the first nucleic acid sequence is an organism that has the same or similar nucleic acid sequence protection properties when processed through the NAT as the organism or organism comprising the target sequence the presence of which is to be tested for in the test article, wherein the protecting vehicle does not comprise the target nucleic acid sequence.

9. The composition of any one of claims 1-8, wherein the protecting vehicle of the first protected nucleic acid sequence is the organism that is being tested for in the NAT without the target nucleic acid sequence and the first nucleic acid sequence is the native genome of the organism that does not comprise the target nucleic acid sequence.

10. The composition of any one of claims 1-9, wherein: (i) the organism comprising the target nucleic acid sequence to be tested for in the NAT is a virulent or pathogenic or toxic organism or wherein when the target nucleic acid sequence is present with or without additional sequences a virulent or pathogenic or toxic trait is conferred on the organism; and (ii) the protecting vehicle of the first protected nucleic acid sequence is a less or non-virulent and/or pathogenic form of the organism comprising a native genomic nucleic acid sequence that does not comprise the target nucleic acid sequence.

11. The composition of any one of claims 1-10, wherein the organism comprising the target nucleic acid sequence to be tested for in the NAT is a particular strain or set of strains belonging to a species or genus of an organism, wherein the protecting vehicle of the first protected nucleic acid sequence is a representative strain of the species or genus comprising a native genomic nucleic acid sequence but not the target nucleic acid sequence.

12. The composition of any one of claims 1-11, wherein the second protected nucleic acid sequence is in a different protecting vehicle to that of the first protected nucleic acid sequence.

13. The composition of any one of claims 1-11, wherein the first and second protected nucleic acid sequences are in one protecting vehicle.

14. The composition of claim 13, wherein the second nucleic acid sequence is recombined into the genomic nucleic acid sequence of its protecting vehicle or is exogenous to the genomic nucleic acid sequence of the protecting vehicle.

15. The composition of any one of claims 1-14, wherein the second nucleic acid sequence is: (i) selected from a portion of the nucleic acid sequence characteristic of the target nucleic acid sequence, or (ii) is modified, such that the second nucleic acid sequence would not be expressed to confer the expression of the genetic feature of the target sequence, either alone or in combination with other nucleic acid sequences in the composition.

16. The composition of any one of claims 1-15, wherein the composition comprises more than one first nucleic acid sequences, and each first nucleic acid sequence being a different nucleic acid sequence characteristic of the organism in the test article, at least one but not necessarily all, of the first nucleic acid sequences being tested for in a NAT.

17. The composition of claim 16, wherein the first nucleic acid sequences overlap.

18. The composition of claim 16, wherein the first nucleic acid sequences do not overlap.

19. The composition of claim 16, 17 or 18, wherein the first nucleic acid sequences are each in a separate protecting vehicle, in more than one protecting vehicles or in one protecting vehicle.

20. The composition of any one of claims 16-19, wherein more than one of the first nucleic acid sequences are tested for in a NAT and the presence of more than one of the first nucleic acid sequences is necessary to determine the presence of the organism.

21. The composition of any one of claims 1-20, wherein the composition comprises, more than one second nucleic acid sequences, each second nucleic acid sequence being different nucleic acid sequences characteristic of the presence of the target nucleic acid sequence, at least one but not necessarily all, of the second nucleic acid sequences being tested for in a NAT.

22. The composition of claim 21, wherein the second nucleic acid sequences overlap.

23. The composition of claim 21, wherein the second nucleic acid sequences do not overlap.

24. The composition of claim 21, 22 or 23, wherein the second nucleic acid sequences are each in a separate protecting vehicle, in more than one protecting vehicles, or in one protecting vehicle.

25. The composition of any one of claims 21-24, wherein more than one of the second nucleic acid sequences are tested for in a NAT and the presence of more than one of the second nucleic acid sequences is necessary to determine the presence of the organism or target nucleic acid sequence, the presence of which is being tested for in the test article by the NAT.

26. The composition of any one of claims 1-25, wherein the first and second nucleic acid sequences are in the same protecting vehicle, said protecting vehicle being less pathogenic or less virulent or less toxic form or having less potential to cause harm compared to the organism suspected to be in the test article.

27. The composition of any one of claims 1-11, wherein the first and second nucleic acid sequences are in different protecting vehicles, the first nucleic acid sequence is in a protecting vehicle which is a less virulent, less pathogenic form of the organism in the test article comprising native nucleic acid sequence and the second nucleic acid sequence is in a second organism as the protecting vehicle that does not comprise a nucleic acid sequence of the first protected nucleic acid sequence.

28. A composition comprising any one of the compositions of claims 1-27, wherein when the NAT is used to detect the presence of one or more additional organisms in a test article, the composition further comprises one or more additional controls for the same or different organism or organisms or target nucleic acid sequence or sequences of the NAT.

29. A composition of claim 28, wherein the one or more additional controls is selected from one or more of:
(a) one or more compositions of claims 1-27, wherein the NAT is used to detect the same or different organism or organisms or target nucleic acid sequence or sequences in the test article;
(b) one or more organisms or other constructs comprising nucleic acid sequences, the presence of which is being tested for in the NAT; and
(c) one or more organisms, antibodies, proteins, lipids, polysaccharides or nucleic acids representing positive control test materials for test being performed concurrently with the NAT.

30. The composition of claim 29, comprising an antibody.

31. The composition of claim 29 or 30, wherein the test sample is being tested for the presence of two organisms, one of which has one nucleic acid sequence to be tested for in a NAT and the other having two nucleic sequences tested for in the NAT, and the quality control composition comprises three organisms, two first protected nucleic acid sequences, and one second protected nucleic acid sequence in a separate protecting vehicle each of the protected nucleic acid sequences detectable in the NAT.

32. The composition of claim 16, wherein the composition comprises three organisms, one first protected nucleic acid sequence in a protecting vehicle that differs from an organism in the test article; and two second protected nucleic acid sequences in separate organisms each comprising nucleic acids not natively present in the vehicle, each second protected nucleic acid sequences being characteristic for the presence of one of the organisms in the test article and each of the protected nucleic acid sequences being subject of NAT.

33. A composition of any one of claims 1-7, wherein the NAT tests for the presence of methicillin-resistant Staphylococcus aureus in the test article, including testing for a nucleic acid sequence that is found generally in a Staphylococcus aureus and for a nucleic acid sequence that is characteristic of the target nucleic acid sequence methicillin resistance, the composition comprising a first and second protected nucleic acid, the first protected nucleic acid comprising a sequence native to Staphylococcus aureus strains that does not express methicillin resistance, and the second protected nucleic acid comprising a sequence characteristic of methicillin resistance, modified or unmodified, but which by itself cannot confer methicillin resistance on its protecting vehicle alone or in combination.

34. The composition of any one of claims 28-33, further comprising at least one antibody epitope tested in an immunoassay, the antibody specific to an organism or substance in the test article.

35. The composition of any one of claims 1-34, wherein the second nucleic acid sequence is in an unprotected vehicle in an amount sufficient to be detected in a NAT despite the loss of a significant quantity of said nucleic acid sequence, wherein the significant loss is greater than for the nucleic acid sequence when protected in the performance of the NAT.

36. The composition of any one of claims 1-35, comprising one or more first nucleic acid sequences alone or together comprises 0.5 to 100% of the genome native to the organism to be tested for in the NAT, or greater than 90%, or greater than 80%, or greater than 70%, or greater than 60%, or greater than 50%, or greater than 40%, or greater than, 30%, or greater than 20%, or greater than 15%, or greater than 10%, or greater than 5%, or greater than 4%, or greater than, 3%, or greater than 2%, or greater than 1%.

37. A method of preparing a composition of any one of claims 1-36, comprising the steps preparing the first and second protected nucleic acid sequences by natural or by genetic modification of the protecting vehicle with the first and second nucleic acid sequences, or by physical or chemical means to couple or include the nucleic acid sequences to or within the protecting vehicle.

38. The method of claim 37, wherein the second nucleic acid sequence is introduced into its protecting vehicle, wherein the protecting vehicle is an organism, with or without integration into the genome sequences of the organism.

39. The method of any one of claim 37 or 38, wherein the second nucleic acid sequence is contained within a vector and introduced into its protecting vehicle by transient transfection, introduced by viral transduction, or integrated into the genome of the organism.

40. The method of claim 39, wherein the second nucleic acid sequence is integrated into the genome by an integrative vector or by Clustered Regularly Interspaced Short Palindromic Repeats (CRISPR), Transcription activator-like effector nucleases (TALEN) or Zinc-finger nucleases (ZFNs) technologies, or by such similar technologies that exist or may arise in the art that facilitate the artificial introduction of nucleic acid sequences into organisms and nucleic acid vectors.

41. A method for assessing the functionality of a NAT, comprising subjecting the quality control composition of any one of claims 1-36 to a NAT and determining if the NAT produces a result that is consistent with the presence of the organism that is the target of the NAT.

42. The method of claim 41 wherein the step of determining comprises determining whether the NAT produces results consistent with the nucleotide sequences known to be present in the quality control composition.

43. The method of claim 41 or 42, further using a pre-determined amount of the quality control composition at different levels to determine the quantitative results of the NAT and/or the detection parameters of the NAT.

44. A method of detecting an organism or target nucleic acid sequence in a test sample using NAT comprising:
(a) obtaining a test sample suspected of comprising the organism or target nucleic acid sequence of interest;
(b) providing a quality control composition according to any one of claims 1-36 optionally in a medium corresponding to a medium of the test article;
(c) performing the Nucleic Acid Tests (NAT) in each of (a) and (b) in parallel or in sequence; and
(d) comparing the results produced by the NAT for the test article with the results produced by the NAT for the quality control composition to assess the functionality of the NAT and to provide supportive evidence for the accuracy of the results of the NAT for the test article.

45. The method of claim 44, wherein the test article medium is selected from the group consisting of whole blood, serum, plasma, defibrinated plasma, stabilized plasma pool, cerebrospinal fluid, urine, saliva, semen, sputum, nasal swab, and vaginal swab, water, buffer solution, transport medium, cell culture medium and fixative medium.

46. A method for detecting an organism or target nucleic acid sequence in the organism in a biological test sample comprising:
(a) obtaining a biological sample suspected of comprising the organism or nucleic acid sequence of interest;
(b) providing a quality control composition according to any one of claims 28 34 in a medium corresponding to a medium of the test sample, and further comprising an expression element or epitope for a characteristic or protein not natively present in the organism used as a protecting vehicle for the first protected nucleic acid sequence;
(c) detecting the organism or nucleic acid sequence of interest and/or the expressed element using Nucleic Acid Tests (NAT);
(d) optionally detecting a protein encoded by the expressed element in an immunoassay or expression based assay; and
(e) comparing the levels of nucleic acids and/or expressed element detected in the biological sample with levels of nucleic acids and/or expressed element detected in the quality control composition.

47. The method of claim 46, wherein the protein(s) in the immunoassay contain at least one antibody epitope that is detected using an antibody.

48. The method of claim 46 or 47, wherein the organism to be detected by the NAT in the test sample is detected simultaneously by both a NAT and an immunoassay.

49. The method of claim 46 or 47, wherein the organism to be detected by the NAT in the test sample is detected sequentially by both a NAT and an immunoassay.

50. The method of any one of claims 46-49, wherein the immunoassay includes an array, microarray formats, plate-based formats, bead-based formats, or gel-based formats.

51. The method of any one of claims 46-50, wherein the control for the immunoassay or expression-based assay is provided in a liquid or as a solid.

52. The composition of any one of claims 1-36 or the method of any one of claims 37-51, wherein the NAT comprises one or more of the following nucleic acid sequence amplification and/or detection techniques, selected from the group consisting of: Polymerase Chain Reaction (PCR), Reverse-transcription Polymerase Chain Reaction (RT-PCR), multiplex PCR or RT-PCR, branched DNA assay, Ligase chain reaction, Transcription Mediated Amplification (TMA), Nucleic Acid Sequence Based Amplification (NASBA), Strand Displacement Amplification (SDA), nucleic acid sequencing, Next-Generation Sequencing (NGS). and such similar technologies that exist or may arise in the art that facilitate the amplification and/or detection of nucleic acid sequences.

53. A kit to assess the functionality of a Nucleic Acid Test (NAT) comprising any one or more of the compositions of claims 1-36 and optionally directions for use.

54. The kit of claim 53, comprising directions for use.

55. The kit of claim 54 wherein the directions for use comprise directions for carrying out the methods of any one or more of claims 37-51.

56. A quality control composition of any one of claims 1-8 used to assess the functionality of a Nucleic Acid Test (NAT) wherein the control is a whole organism control composition comprising a whole organism as a protecting vehicle, the whole organism comprising: i) at least one first protected nucleic acid sequence with a first nucleic acid sequence that provides a characteristic of that organism that is tested in a NAT, and ii) at least one second protected nucleic acid sequence with a second nucleic acid sequence providing a characteristic not natively present in the organism that is tested in a NAT.

57. A quality control composition of any one of claims 1-8 used to assess the functionality of a Nucleic Acid Test (NAT) wherein the control is a whole organism control composition comprising a whole organism protecting vehicle comprising: i) a first nucleic acid sequence providing a characteristic not natively present in the organism tested in a Nucleic Acid Test (NAT), and ii) the second nucleic acid sequence providing a second characteristic not natively present in the organism and is tested in a NAT.

58. A quality control composition of any one of claims 1-8 used to assess the functionality of a Nucleic Acid Test (NAT) wherein the control is a whole organism control composition comprising two or more whole organisms as protecting vehicles wherein the composition comprises: i) a first intact organism containing at least one nucleic acid sequence that is a characteristic of that organism tested in a Nucleic Acid Test (NAT) as a first protected nucleotide sequence, and ii) at least a second organism containing a second nucleic acid sequence providing a characteristic not natively present in the first or second organism and is tested in a NAT as a second protected nucleic acid sequence.

59. The composition of any one of claims 56-58, wherein the whole organism protecting vehicle has the same or similar nucleic acid sequence protection properties when processed through the NAT as the organism or organism comprising the target nucleic acid sequence the presence of which is to be tested for in the test article, wherein the protecting vehicle does not comprise the target nucleic acid sequence.

60. The composition of any one of claims 56-59, wherein the not natively present nucleic acid sequence is introduced into the whole organism by natural or by genetic modification with or without integration into the genome sequences of the organism.

61. The composition of any one of claims 56-60, wherein the not natively present nucleic acid sequence is contained within a vector, introduced into the organism by transient transfection, introduced by viral transduction, or integrated into the genome of the organism.

62. The composition of claim 60, wherein the not natively present nucleic acid sequence is integrated into the genome of the whole organism by an integrative vector or by Clustered Regularly Interspaced Short Palindromic Repeats (CRISPR), Transcription activator-like effector nucleases (TALEN) or Zinc-finger nucleases (ZFNs) technologies, or by such similar technologies that exist or may arise in the art that facilitate the artificial introduction of nucleic acid sequences into organisms and nucleic acid vectors.

63. The composition of any one of claims 56-62, wherein the not natively present nucleic acid sequence encodes all or part or a target nucleic acid sequence that is a variant of a gene to be tested in a NAT.

64. The composition of any one of claims 56-63, wherein the NAT comprises one or more of the following nucleic acid sequence amplification and/or detection techniques, selected from the group consisting of: Polymerase Chain Reaction (PCR), Reverse-transcription Polymerase Chain Reaction (RT-PCR), multiplex PCR or RT-PCR, branched DNA assay, Ligase chain reaction, Transcription Mediated Amplification (TMA), Nucleic Acid Sequence Based Amplification (NASBA), Strand Displacement Amplification (SDA), nucleic acid sequencing, Next-Generation Sequencing (NGS). and such similar technologies that exist or may arise in the art that facilitate the amplification and/or detection of nucleic acid sequences.

65. The composition of any one of claims 56-64, wherein the organisms are selected from the group consisting of: bacteria, viruses, fungi, archaea, protists, parasites, and eukaryotic cells.

66. The composition of claim 58, comprising two or more organisms as first and second protecting vehicles, wherein the organisms may be the same species or different species within the organism family and optionally the composition comprising additional nucleic acid comprising vehicles comprising nucleic acid sequences characteristic of a nucleic acid in a test article to be tested for in the NAT.

67. The composition of claim 58 or 66, wherein when the composition comprises two or more organisms, the first and second or additional organisms as protecting vehicles for the protected nucleic acid sequences may be of the same phylogenetic lineage or of different phylogenetic lineages.

68. The composition of claim 67, wherein the first organism is a eukaryote and the second or additional organism is a eukaryote, bacterium, virus, fungi, archaea, protist, parasite and the composition optionally comprising additional nucleic acid comprising vehicles, the nucleic acid sequences of which are tested for in the NAT, the vehicle being a plasmid, or liposome, or vesicle, or protein capsid or shell, or microcompartment, or nanovesicle, or exosome, or micelle, or solid lipid nanoparticle, or lipid-coated particle, or nanotube, or nanocrystal, or polymeric nanoparticle, or inorganic nanoparticle, or interpolyelectrolyte complex (polyplex), or dendrimer.

69. The composition of claim 67, wherein the first organism is a bacterium and the second or additional organism is a eukaryote, bacterium, virus, fungi, archaea, protist, parasite and the composition optionally comprising additional nucleic acid comprising vehicles, the nucleic acid sequences of which are tested for in the NAT, the vehicle being a plasmid, or liposome, or vesicle, or protein capsid or shell, or microcompartment, or nanovesicle, or exosome, or micelle, or solid lipid nanoparticle, or lipid-coated particle, or nanotube, or nanocrystal, or polymeric nanoparticle, or inorganic nanoparticle, or interpolyelectrolyte complex (polyplex), or dendrimer.

70. The composition of claim 67, wherein the first organism is a virus and the second or additional organism is a eukaryote, bacterium, virus, fungi, archaea, protist, or parasite, and the composition optionally comprising additional nucleic acid comprising vehicles, the nucleic acid sequences of which are tested for in the NAT, the vehicle being a plasmid, or liposome, or vesicle, or protein capsid or shell, or microcompartment, or nanovesicle, or exosome, or micelle, or solid lipid nanoparticle, or lipid-coated particle, or nanotube, or nanocrystal, or polymeric nanoparticle, or inorganic nanoparticle, or interpolyelectrolyte complex (polyplex), or a dendrimer.

71. The composition of claim 67, wherein the first organism is a fungi and the second or additional organism is a eukaryote, bacterium, virus, fungi, archaea, protist, parasite and the composition optionally comprising additional nucleic acid comprising vehicles, the nucleic acid sequences of which are tested for in the NAT, the vehicle being a plasmid, or liposome, or vesicle, or protein capsid or shell, or microcompartment, or nanovesicle, or exosome, or micelle, or solid lipid nanoparticle, or lipid-coated particle, or nanotube, or nanocrystal, or polymeric nanoparticle, or inorganic nanoparticle, or interpolyelectrolyte complex (polyplex), or dendrimer.

72. The composition of claim 67, wherein the first organism is an archaea, and the second or additional organism is a eukaryote, bacterium, virus, fungi, archaea, protist, parasite, and the composition optionally comprising additional nucleic acid comprising vehicles, the nucleic acid sequences of which are tested for in the NAT, the vehicle being a plasmid, or liposome, or vesicle, or protein capsid or shell, or microcompartment, or nanovesicle, or exosome, or micelle, or solid lipid nanoparticle, or lipid-coated particle, or nanotube, or nanocrystal, or polymeric nanoparticle, or inorganic nanoparticle, or interpolyelectrolyte complex (polyplex), or dendrimer.

73. The composition of claim 67, wherein the first organism is a protist, and the second or additional organism is a eukaryote, bacterium, virus, fungi, archaea, protist, parasite, and the composition optionally comprising additional nucleic acid comprising vehicles, the nucleic acid sequences of which are tested for in the NAT, the vehicle being a plasmid, or liposome, or vesicle, or protein capsid or shell, or microcompartment, or nanovesicle, or exosome, or micelle, or solid lipid nanoparticle, or lipid-coated particle, or nanotube, or nanocrystal, or polymeric nanoparticle, or inorganic nanoparticle, or interpolyelectrolyte complex (polyplex), or dendrimer.

74. The composition of claim 67, wherein the first organism is a parasite, and the second or additional organism is a eukaryote, bacterium, virus, fungi, archaea, protist, parasite, and the composition optionally comprising additional nucleic acid comprising vehicles, the nucleic acid sequences of which are tested for in the NAT, the vehicle being a plasmid, or liposome, or vesicle, or protein capsid or shell, or microcompartment, or nanovesicle, or exosome, or micelle, or solid lipid nanoparticle, or lipid-coated particle, or nanotube, or nanocrystal, or polymeric nanoparticle, or inorganic nanoparticle, or interpolyelectrolyte complex (polyplex), or dendrimer.

75. The composition of claim 66, wherein if the organism is a bacterium, the first organism and the second or additional organism is the same bacterial species or different bacterial species.

76. The composition of claim 66, wherein if the organism is a virus, the first organism and the second or additional organism is the same virus species or different virus species.

77. The composition of claim 66, wherein the first organism is a eukaryotic cell and the second or additional organism is bacterium.

78. The composition of any one of claims 65-77, wherein the bacterium is selected from the group consisting of: Staphylococcus, Staphylococcus Aureus, Streptococcus, Streptococcus pneumoniae, Streptococcus pyogenes, Gonorrhea, Neisseria gonorrhoeae, Enterococcus, Mycobacterium tuberculosis, and Escherichia coli.

79. The composition of any one of claims 65-77, wherein the virus is selected from the group consisting of: human immunodeficiency virus, hepatitis C virus, hepatitis B virus, cytomegalovirus, baculovirus, human lymphotrophic virus, Epstein-Barr virus, parvovirus, herpes simplex virus, human herpes virus 8 and hepatitis A Virus.

80. The composition of any one of claims 65-77, wherein the fungus is selected from the group consisting of: Candida albicans, Aspergillus fumigatus, and Cryptococcus neoformans, non-fumigatus Aspergillus, Zygomycetes, Fusarium and Pseudallescheria boydii.

81. The composition of any one of claims 65-77, wherein the archaea is selected from the group consisting of: Euryarchaeota, Crenarchaeota, Nanoarchaeota, Thaumarchaeota, Aigarchaeota, Crenarchaeota, Korarchaeota, Metallosphera sedula, Methanobacterium thermoautotrophicum, Methanococcus jannaschii, Methanococcus jannaschii, Pyrococcus abyssi ST549, Pyrococcus furiosus, Pyrococcus furiosus, Pyrococcus furiosus, Pyrococcus furiosus, Sulfolobus shibatae, Sulfolobus solfataricus Gθ, Sulfolobus solfataricus MT4, Thermococcusbarosii, and Thermococcus litoralis.

82. The composition of any one of claims 65-77, wherein the protist is selected from the group consisting of Amoeba proteus, Euglena gracilis, Paramecium Aurelia, Plasmodium falciparum, and Pediastrum boryanum.

83. The composition of any one of claims 65-77, wherein the parasite is selected from the group consisting of Ectoparasites, Endoparasites, Mesoparasites, Parasitoids, Hyperparasites, Social parasites, and Adelpho-parasites.

84. The composition of any one of claims 65-77, wherein the eukaryotic cell is selected from the group consisting of Chinese hamster ovary (CHO, CHO-K1, CHO pro-3, DUKX-X11, DG44), Human embryonic kidney 293 (HEK 293, HEK 293T), Madin-Darby canine kidney (MDCK), mouse myeloma cells (NS0, Sp2/0 or others), Murine erythroleukemia (MEL), african green monkey kidney (COS, Vero, or others) cells, insect cells (Spodoptera frugiperda, Sf9, Sf21, Trichoplusia ni, or others), and yeast cells (Pichia pastoris, Saccharomyces cerevisiae, or others).

85. The composition of any one of claims 56-84, wherein the Whole Organism Control comprises a) a wild type first organism without any genetic modification, and a genetic sequence or sequences that are the target of a NAT; and b) a second organism that includes a modified nucleic acid sequence, that is also detected by a NAT.

86. The composition of any one of claims 56-85, wherein the Whole Organism Control comprises a) a wild type first organism without any genetic modification, and a genetic sequence or sequences that are the target of a NAT; and b) a second organism that is also detected by a NAT.

87. The composition of any one of claims 56-86, wherein the second nucleic acid sequence encodes a gene selected from the group consisting of an antibiotic resistance gene, a drug resistance gene, a biomarker, an immune evasion marker, a virus gene, an oncogene, or a tumor suppressor gene.

88. The composition of claim 87, wherein the antibiotic resistance gene is selected from the group consisting of: aac2ia, aac2ib, aac2ic, aac2id, aac2i, aac3ia, aac3iia, aac3iib, aac3iii, aac3iv, aac3ix, aac3vi, aac3viii, aac3vii, aac3x, aac6i, aac6ia, aac6ib, aac6ic, aac6ie, aac6if, aac6ig, aac6iia, aac6iib, aad9, aad9ib, aadd, acra, acrb, adea, adeb, adec, amra, amrb, ant2ia, ant2ib, ant3ia, ant4iia, ant6ia, aph33ia, aph33ib, aph3ia, aph3ib, aph3ic, aph3iiia, aph3iva, aph3va, aph3vb, aph3via, aph3viia, aph4ib, aph6ia, aph6ib, aph6ic, aph6id, arna, baca, bcra, bcrc, bl1_acc, bl1_ampc, bl1_asba, bl1_ceps, bl1_cmy2, bl1_ec, bl1_fox, bl1_mox, bl1_och, bl1_pao, bl1_pse, bl1_sm, bl2a_1, bl2a_exo, bl2a_iii2, bl2a_iii, bl2a_kcc, bl2a_nps, bl2a_okp, bl2a_pc, bl2be_ctxm, bl2be_oxy1, bl2be_per, bl2be_shv2, bl2b_rob, bl2b_tem1, bl2b_tem2, bl2b_tem, bl2b_tle, bl2b_ula, bl2c_bro, bl2c_pse1, bl2c_pse3, bl2d_lcr1, bl2d_moxa, bl2d_oxa10, bl2d_oxa1, bl2d_oxa2, bl2d_oxa5, bl2d_oxa9, bl2d_r39, bl2e_cbla, bl2e_cepa, bl2e_cfxa, bl2e_fpm, bl2e_y56, bl2f_nmca, bl2f_sme1, bl2_ges, bl2_kpc, bl2_len, bl2_veb, bl3_ccra, bl3_cit, bl3_cpha, bl3_gim, bl3_imp, bl3_l, bl3_shw, bl3_sim, bl3_vim, ble, blt, bmr, cara, cata10, cata11, cata12, cata13, cata14, cata15, cata16, cata1, cata2, cata3, cata4, cata5, cata6, cata7, cata8, cata9, catb1, catb2, catb3, catb4, catb5, ceoa, ceob, cml_e1, cml_e2, cml_e3, cml_e4, cml_e5, cml_e6, cml_e7, cml_e8, dfra10, dfra12, dfra13, dfra14, dfra15, dfra16, dfra17, dfra19, dfra1, dfra20, dfra21, dfra22, dfra23, dfra24, dfra25, dfra25, dfra25, dfra26, dfra5, dfra7, dfrb1, dfrb2, dfrb3, dfrb6, emea, emrd, emre, erea, ereb, erma, ermb, ermc, ermd, erme, ermf, ermg, ermh, ermn, ermo, ermq, ermr, erms, ermt, ermu, ermv, ermw, ermx, ermy, fosa, fosb, fosc, fosx, fusb, fush, ksga, lmra, lmrb, lnua, lnub, Isa, maca, macb, mdte, mdtf, mdtg, mdth, mdtk, mdtl, mdtm, mdtn, mdto, mdtp, meca, mecr1, mefa, mepa, mexa, mexb, mexc, mexd, mexe, mexf, mexh, mexi, mexw, mexx, mexy, mfpa, mpha, mphb, mphc, msra, norm, oleb, opcm, opra, oprd, oprj, oprm, oprn, otra, otrb, pbp1a, pbp1b, pbp2b, pbp2, pbp2x, pmra, qac, qaca, qacb, qnra, qnrb, qnrs, rosa, rosb, smea, smeb, smec, smed, smee, smef, srmb, sta, str, sul1, sul2, sul3, tcma, tcr3, tet30, tet31, tet32, tet33, tet34, tet36, tet37, tet38, tet39, tet40, teta, tetb, tetc, tetd, tete, tetg, teth, tetj, tetk, tetl, tetm, teto, tetpa, tetpb, tet, tetq, tets, tett, tetu, tetv, tetw, tetx, tety, tetz, tlrc, tmrb, tolc, tsnr, vana, vanb, vanc, vand, vane, yang, vanha, vanhb, vanhd, vanra, vanrb, vanrc, vanrd, vanre, vanrg, vansa, vansb, vansc, vansd, vanse, vansg, vant, vante, vantg, vanug, vanwb, vanwg, vanxa, vanxb, vanxd, vanxyc, vanxye, vanxyg, vanya, vanyb, vanyd, vanyg, vanz, vata, vatb, vatc, vatd, vate, vgaa, vgab, vgba, vgbb, vph, ykkc, and ykkd.

89. The composition of any one of claims 56-88, wherein the first organism is Staphylococcus aureus and the second organism is Escherichia Coli, wherein the Escherichia Coli has been modified to contain all or a portion of the mecA gene.

90. The composition of any one of claims 1-89, wherein the organism or protecting vehicle is inactivated, optionally the inactivation is by formalin, aldehydes and other chemicals, gamma irradiation, UV irradiation, dehydration, X-ray, heat, or detergent.

91. A composition comprising a Whole Organism Control wherein the Whole Organism Control comprises: i) a whole organism containing at least one nucleic acid sequence providing a characteristic of that organism tested in a NAT, and ii) a sufficient amount of free nucleic acid providing a characteristic not natively present in the organism, which characteristic is tested in a NAT.

92. A composition comprising Whole Organism Control wherein the Whole Organism Control comprises: i) a whole organism containing at least one nucleic acid sequence providing a characteristic not natively present in that organism tested in a NAT, and ii) a sufficient amount of free nucleic acid that is a second nucleic acid sequence providing a characteristic not natively present in the organism, which characteristic is tested in a NAT.

93. The composition of claim 91 or 92, wherein the not natively present nucleic acid sequence encodes all or part of a gene to be tested in a NAT.

94. The composition of any one of claims 91-93, wherein Nucleic Acid Tests (NAT) comprises one or more of the following nucleic acid sequence amplification and/or detection techniques, selected from the group consisting of: Polymerase Chain Reaction (PCR), Reverse-transcription Polymerase Chain Reaction (RT-PCR), multiplex PCR or RT-PCR, branched DNA assay, Ligase chain reaction, Transcription Mediated Amplification (TMA), Nucleic Acid Sequence Based Amplification (NASBA), Strand Displacement Amplification (SDA), nucleic acid sequencing, Next-Generation Sequencing (NGS), and such similar technologies that exist or may arise in the art that facilitate the amplification and/or detection of nucleic acid sequences.

95. The composition of any one of claims 91-94, wherein the organism is selected from the group consisting of bacteria, viruses, fungi, archaea, protists, parasites, eukaryotic cells, and the composition optionally comprising additional nucleic acid comprising vehicles, the nucleic acid sequences of which are tested for in the NAT, the vehicle being a plasmids, or liposomes, or vesicles, or protein capsids or shells, or microcompartments, or nanovesicles, or exosomes, or micelles, or solid lipid nanoparticles, or lipid-coated particles, or nanotubes, or nanocrystals, or polymeric nanoparticles, or inorganic nanoparticles, or interpolyelectrolyte complexes (polyplexes), or dendrimers.

96. A method for detecting an organism or nucleic acid sequence in a biological test sample comprising:
(a) obtaining a biological sample suspected of comprising the organism or nucleic acid sequence of interest;
(b) providing a Whole Organism Control according to any one of claims 56-95 comprising an organism in a medium corresponding to a medium of the test sample;
(c) detecting the organism or nucleic acid sequence of interest using Nucleic Acid Tests (NAT); and
(d) comparing the levels of nucleic acids detected in the biological sample with the levels of nucleic acids detected in the Whole Organism Control.

97. A method for detecting an organism or nucleic acid sequence in a biological test sample comprising:
(a) obtaining a biological sample suspected of comprising the organism or nucleic acid sequence of interest;
(b) providing a Multi-Organism Control comprising two or more whole organisms in a medium corresponding to a medium of the test sample;
(c) detecting the organism or nucleic acid sequence of interest using Nucleic Acid Tests (NAT); and
(d) comparing the levels of nucleic acids detected in the biological sample with levels of nucleic acids detected in the Control.

98. The method of claim 96 or 97, wherein the medium test sample is selected from the group consisting of whole blood, serum, plasma, defibrinated plasma, stabilized plasma pool, cerebrospinal fluid, urine, saliva, semen, sputum, nasal swab, and vaginal swab.

99. A kit comprising Whole Organism Controls of any one of claims 56-94, and instructions for use.

100. The composition of any one of claims 56-94, wherein the Whole Organism Control comprises a nucleic acid sequence that encodes and expresses a protein(s) capable of detection in an immunoassay and wherein the Whole Organism Control can be used in both a NAT and an immunoassay.

101. The composition of claim 100, wherein the nucleic acid sequence encodes a gene selected from the group consisting of: an antibiotic resistance gene, a drug resistance gene, a biomarker, an antigen, an immune evasion marker, a virus gene, an oncogene, or a tumor suppressor gene wherein the expressed protein is detectable in an immunoassay.

102. A method for detecting an organism or nucleic acid sequence in a biological test sample comprising:
(a) obtaining a biological sample suspected of comprising the organism or nucleic acid sequence of interest;
(b) providing a Whole Organism Control according to any one of claims 56-95 comprising a Whole Organism in a medium corresponding to a medium of the test sample, and further comprising an expression element for a characteristic or protein not natively present in the Whole Organism Control;
(c) detecting the organism or nucleic acid sequence of interest and/or the expressed element using Nucleic Acid Tests (NAT);
(d) optionally detecting a protein encoded by the expressed element in an immunoassay or expression-based assay; and
(e) comparing the levels of nucleic acids and/or expressed element detected in the biological sample with levels of nucleic acids and/or expressed element detected in the Whole Organism Control.

103. The method of claim 102, wherein the protein(s) in the immunoassay contain at least one antibody epitope that is detected using an antibody.

104. The method of claim 102 or 103, wherein the Whole Organism Control is detected simultaneously by both a NAT and an immunoassay.

105. The method of claim 102 or 103, wherein the Whole Organism Control is detected sequentially by both a NAT and an immunoassay.

106. The method of any one of claims 102-105, wherein the immunoassay includes an array, microarray formats, plate-based formats, bead-based formats, or gel-based formats.

107. The method of any one of claims 102-106, wherein the Control for the immunoassay or expression-based assay is provided in a liquid or as a solid.

108. A composition comprising a Whole Organism Control for use in a Nucleic Acid Test (NAT) wherein the Whole Organism Control comprises:
(a) a less virulent or less pathogenic or less toxic version of the organism for which the sample is to be screened, which is less toxic or less pathogenic or less virulent or dangerous than the organism to be screened for in the NAT; and
(b) one or more nucleotide sequences that are determinative of the presence of the virulent, pathogenic or toxic strain of a whole organism containing i) at least one nucleic acid sequence providing a characteristic of that organism that is tested in a Nucleic Acid Test (NAT), and ii) containing a second nucleic acid sequence providing a characteristic not natively present in the organism that is tested in a NAT.
